(19) United States
(12) Patent Application Publication
(10) Pub. No.: US 2017/0143259 A1
(43) Pub. Date: May 25, 2017

(54) DETECTING AND TREATING DISORDERED BREATHING

(71) Applicant: INVICTA MEDICAL, INC., Portola Valley, CA (US)

(72) Inventors: Steven Thomas Kent, Portola Valley, CA (US); Harold Byron Kent, Portola Valley, CA (US); Christopher Fisher, Santa Clara, CA (US); Ronald W. Young, Los Altos, CA (US); Karena Yadira Puldon, Northridge, CA (US); Robert Douglas Kent, Portola Valley, CA (US); Laurence Wylie Harter, San Jose, CA (US)

(21) Appl. No.: 15/390,450

(22) Filed: Dec. 23, 2016

Related U.S. Application Data
(63) Continuation-in-part of application No. 14/149,689, filed on Jan. 7, 2014.
(60) Provisional application No. 62/387,428, filed on Dec. 23, 2015.
(60) Provisional application No. 62/387,464, filed on Dec. 23, 2015.
(60) Provisional application No. 62/387,427, filed on Dec. 23, 2015.
(60) Provisional application No. 62/387,463, filed on Dec. 23, 2015.
(60) Provisional application No. 62/387,507, filed on Dec. 23, 2015.
(60) Provisional application No. 62/387,395, filed on Dec. 24, 2015.

(51) Int. Cl. A61B 5/00; A61N 1/36; A61B 5/087; A61N 1/05; A61B 5/08; A61B 5/0488

(52) U.S. Cl. CPC A61B 5/4836; A61B 5/682; A61B 5/0816; A61B 5/0488; A61B 5/4833; A61B 5/087; A61N 1/0548; A61N 1/3601

(57) ABSTRACT

A device is disclosed that can detect and treat disordered breathing in a patient. The device can include at least one contact adapted to make contact with a portion of an oral cavity of a patient, and a control circuit configured to: in a first mode, detect a presence of disordered breathing in the patient based, at least in part, on a first signal received from the at least one contact; and in a second mode, provide electrical stimulation via the at least one contact to a portion of the patient's upper airway based on a detection of the presence of disordered breathing.

### CROSS-REFERENCE TO RELATED APPLICATIONS

This application is a continuation-in-part of and claims priority to co-pending and commonly owned U.S. patent application Ser. No. 14/149,689 entitled “METHOD AND APPARATUS FOR TREATING SLEEP APNEA” filed on Jan. 7, 2014, the entirety of which is incorporated by reference herein. This application also claims priority under 35 USC 119(e) to co-pending and commonly owned U.S. Provisional Patent Application No. 62/387,428 entitled “METHOD AND APPARATUS FOR PREDICTING DISORDERED BREATHING” filed on Dec. 23, 2015, to co-pending and commonly owned U.S. Provisional Patent Application No. 62/387,464 entitled “METHOD AND APPARATUS FOR DETECTING AND TREATING SNORING” filed on Dec. 23, 2015, co-pending and commonly owned U.S. Provisional Patent Application No. 62/387,427 entitled “METHOD AND APPARATUS FOR MONITORING RESPIRATION” filed on Dec. 23, 2015, co-pending and commonly owned U.S. Provisional Patent Application No. 62/387,463 entitled “METHOD AND APPARATUS FOR DETECTING AND TREATING APNEA” filed on Dec. 23, 2015, co-pending and commonly owned U.S. Provisional Patent Application No. 62/387,507 entitled “METHOD AND APPARATUS FOR DETERMINING A LEVEL OF CONSCIOUSNESS” filed on Dec. 23, 2015, co-pending and commonly owned U.S. Provisional Patent Application No. 62/387,395 entitled “METHOD AND APPARATUS FOR SENSING SLEEP LEVELS” filed on Dec. 24, 2015, the entireties of all of which are incorporated by reference herein.

### TECHNICAL FIELD

The present embodiments relate generally to disturbed or disordered breathing in patients, and specifically to non-invasive techniques for predicting the onset of disordered breathing, detecting the occurrence of disordered breathing, and providing therapy for disordered breathing.

### BACKGROUND OF RELATED ART

Obstructive sleep apnea (OSA) is a medical condition in which a patient's upper airway is repeatedly partially or fully occluded during sleep. These repeated occlusions of the upper airway may cause sleep fragmentation, which in turn may result in sleep deprivation, daytime tiredness, malaise, weakening of the immune system, reduction in cognitive function, hypertension, high blood pressure, and other undesirably conditions. More serious instances of OSA may increase the patient's risk for stroke, cardiac arrhythmias, high blood pressure, and/or other disorders.

OSA may be characterized by the tendency of the soft tissues of the upper airway to collapse during sleep, thereby occluding the upper airway. More specifically, OSA is typically caused by the collapse of the patient's soft palate and/or by the collapse of the patient's tongue (e.g., onto the back of the pharynx), which in turn may obstruct normal breathing.

There are many treatments available for OSA including, for example: surgery, constant positive airway pressure (CPAP) machines, and the electrical stimulation of muscles associated with moving the tongue. Surgical techniques include tracheotomies, procedures to remove portions of a patient's tongue and/or soft palate, and other procedures that seek to prevent collapse of the tongue into the back of the pharynx. These surgical techniques are very invasive. CPAP machines seek to maintain upper airway patency by applying positive air pressure at the patient's nose and mouth. However, these machines are uncomfortable and may have low compliance rates.

Some electrical stimulation techniques seek to prevent collapse of the tongue into the back of the pharynx by causing the tongue to protrude forward (e.g., in an anterior direction) during sleep. For one example, U.S. Pat. No. 4,830,008 to Meer discloses an invasive technique in which electrodes are implanted into a patient at locations on or near nerves that stimulate the Genioglossus muscle to move the tongue forward (e.g., away from the back of the pharynx). For another example, U.S. Pat. No. 7,711,438 to Lattner discloses a non-invasive technique in which electrodes, mounted on an intraoral device, electrically stimulate the Genioglossus muscle to cause the tongue to move forward during respiratory inspiration. In addition, U.S. Pat. No. 8,359,108 to McCreery teaches an intraoral device that applies electrical stimulation to the Hypoglossal nerve to contract the Genioglossus muscle, which as mentioned above may prevent tongue collapse by moving the tongue forward during sleep.

Moving a patient's tongue forward during sleep may cause the patient to wake, which is not desirable. In addition, existing techniques for electrically stimulating the Hypoglossal nerve and/or the Genioglossus muscle may cause discomfort and/or pain, which is not desirable. Further, invasive techniques for electrically stimulating the Hypoglossal nerve and/or the Genioglossus muscle undesirably require surgery and introduce foreign matter into the patient's tissue, which is undesirable. Thus, there is a need for a non-invasive treatment for OSA that does not disturb or wake-up the patient during use.

In addition, it would be desirable to be able to predict or detect the onset of disordered breathing in a patient, and provide treatment that eliminates (or at least reduce the severity of) disordered breathing in the patient.

### BRIEF DESCRIPTION OF THE DRAWINGS

The present embodiments are illustrated by way of example and are not intended to be limited by the figures of the accompanying drawings, where like reference numerals refer to corresponding parts throughout the drawing figures.

FIG. 1A is a side sectional view depicting a patient's upper airway.

FIG. 1B is a front plan view of the patient's oral cavity.

FIG. 1C is an elevated sectional view of the patient's tongue.

FIG. 1D is a side sectional view of the patient's tongue.

FIG. 2A is a top plan view of a device, situated over a patient's lower teeth, in accordance with some embodiments.

FIG. 2B is an elevated perspective view of the device of FIG. 2A.

FIG. 2C is a top plan view of a device, situated over a patient's lower teeth, in accordance with other embodiments.

FIG. 2D is an elevated perspective view of the device of FIG. 2C.

FIG. 3A is a side sectional view depicting a patient's upper airway during disturbed breathing.

FIG. 3B is a side sectional view depicting the patient's upper airway in response to electrical stimulation provided in accordance with the example embodiments.

FIG. 4 is a block diagram of the electrical components of the device of FIGS. 2A-2B.

FIG. 5 is a circuit diagram illustrating an electrical model of the patient's tongue.

FIG. 6 is an illustrative flow chart depicting an example operation in accordance with some embodiments.

FIG. 7A is an elevated perspective view of a device in accordance with other embodiments.

FIG. 7B is an elevated perspective view of the device of FIG. 7A situated over a patient's teeth.

FIG. 7C is a rear plan view of the device of FIG. 7A situated over a patient's teeth.

FIG. 7D is a front plan view of the device of FIG. 7A situated over a patient's teeth.

FIG. 8A is a top plan view of a device, shown to be inserted within a patient's oral cavity, configured to monitor one or more states of the patient's upper airway in accordance with the example embodiments.

FIG. 8B is a block diagram of the control circuit of the example device of FIG. 8A.

FIG. 9A is an illustrative graph of example signals indicating normal breathing of a patient, in accordance with some embodiments.

FIG. 9B is an illustrative graph of example signals depicting a transition from disordered breathing to an arousal to normal breathing of a patient, in accordance with some embodiments.

FIG. 9C is an illustrative graph of example signals indicating an onset of snoring of a patient, in accordance with some embodiments.

FIG. 9D is an illustrative graph of example signals indicating an onset of apnea of a patient, in accordance with some embodiments.

FIG. 9E is an illustrative graph of example signals indicating an onset of CNS depression of a patient, in accordance with some embodiments.

FIG. 10A is an illustrative graph depicting signals indicating normal breathing in a patient, in accordance with some embodiments.

FIG. 10B is an illustrative graph depicting signals indicating an onset snoring in a patient, in accordance with some embodiments.

FIG. 10C is an illustrative graph depicting signals indicating an onset of hypopnea in a patient, in accordance with some embodiments.

FIG. 10D is an illustrative graph depicting signals indicating an onset of obstructive apnea in a patient, in accordance with some embodiments.

FIG. 11A is an illustrative flow chart depicting an example operation for detecting and treating disordered breathing in a patient, in accordance with some embodiments.

FIG. 11B is an illustrative flow chart depicting an example operation for detecting an onset of apnea or disordered breathing in a patient, in accordance with some embodiments.

FIG. 11C is an illustrative flow chart depicting an example operation for monitoring a respiration of a patient, in accordance with some embodiments.

FIG. 11D is an illustrative flow chart depicting an example operation for detecting an onset of snoring of a patient, in accordance with some embodiments.

FIG. 11E is an illustrative flow chart depicting an example operation for determining a level of sleep or a level of consciousness of a patient, in accordance with some embodiments.

FIG. 11F is an illustrative flow chart depicting an example operation for determining a level of compliance of a patient, in accordance with some embodiments.

FIG. 11G is an illustrative flow chart depicting an example operation for determining a type of disordered breathing in a patient, in accordance with some embodiments.

### DETAILED DESCRIPTION

A non-invasive method and apparatus for treating sleep disorders, such as obstructive sleep apnea (OSA) and/or snoring, are disclosed herein. In the following description, numerous specific details are set forth to provide a thorough understanding of the present disclosure. Also, in the following description and for purposes of explanation, specific nomenclature is set forth to provide a thorough understanding of the present embodiments. However, it will be apparent to one skilled in the art that these specific details may not be required to practice the present embodiments. In other instances, well-known circuits and devices are shown in block diagram form to avoid obscuring the present disclosure. The term “coupled” as used herein means connected directly to or connected through one or more intervening components, circuits, or physiological matter. Any of the signals provided over various buses described herein may be time-multiplexed with other signals and provided over one or more common buses, or may be wirelessly transmitted between a number of component, circuits, sensors, and/or devices of the example embodiments. Additionally, the interconnection between circuit elements or software blocks may be shown as buses or as single signal lines. Each of the buses may alternatively be a single signal line, and each of the single signal lines may alternatively be buses, and a single line or bus might represent any one or more of a myriad of physical or logical mechanisms for communication between components. Further, the logic levels and timing assigned to various signals in the description below are arbitrary and/or approximate, and therefore may be modified (e.g., polarity reversed, timing modified, etc) as desired.

The terminology used herein is for the purpose of describing particular aspects only and is not intended to be limiting of the aspects. As used herein, the singular forms “a,” “an” and “the” are intended to include the plural forms as well, unless the context clearly indicates otherwise. It will be further understood that the terms “comprises,” “comprising,” “includes” or “including,” when used herein, specify the presence of stated features, integers, steps, operations, elements, or components, but do not preclude the presence or addition of one or more other features, integers, steps, operations, elements, components, or groups thereof. Moreover, it is understood that the word “or” has the same meaning as the Boolean operator “OR,” that is, it encompasses the possibilities of “either” and “both” and is not limited to “exclusive or” (“XOR”), unless expressly stated otherwise. It is also understood that the symbol “I” between two adjacent words has the same meaning as “or” unless expressly stated otherwise. Moreover, phrases such as “connected to,” “coupled to” or “in communication with” are not limited to direct connections unless expressly stated otherwise. In addition, the term “detecting” as used herein may also mean “observing” and “monitoring,” and the term “determining” as used herein may also mean “analyzing,” “considering,” evaluating,” and/or “interpreting.”

As used herein, the term “substantially lateral direction” refers to a direction across the patient's oral cavity in which the direction's lateral components are larger than the direction's anterior-to-posterior components (e.g., a substantially lateral direction may refer to any direction that is less than approximately 45 degrees from the lateral direction, as defined below with respect to the drawing figures). Further, as used herein, the term “reversible current” means a current that changes or reverses polarity from time to time between two controllable voltage potentials.

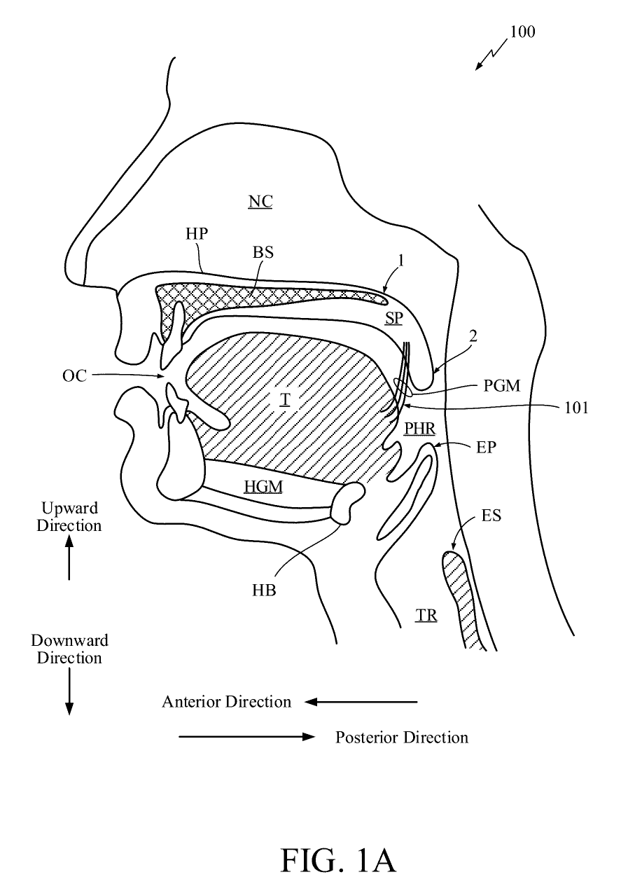

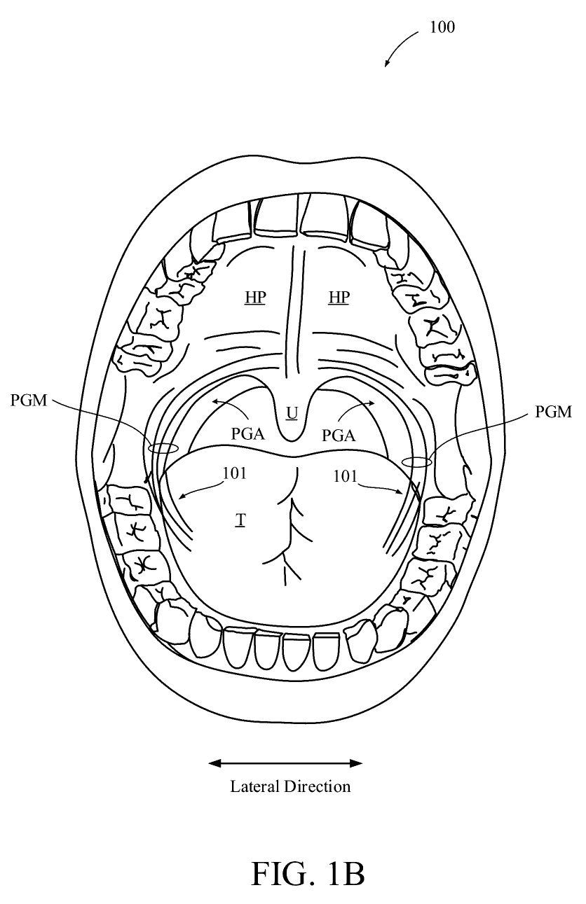

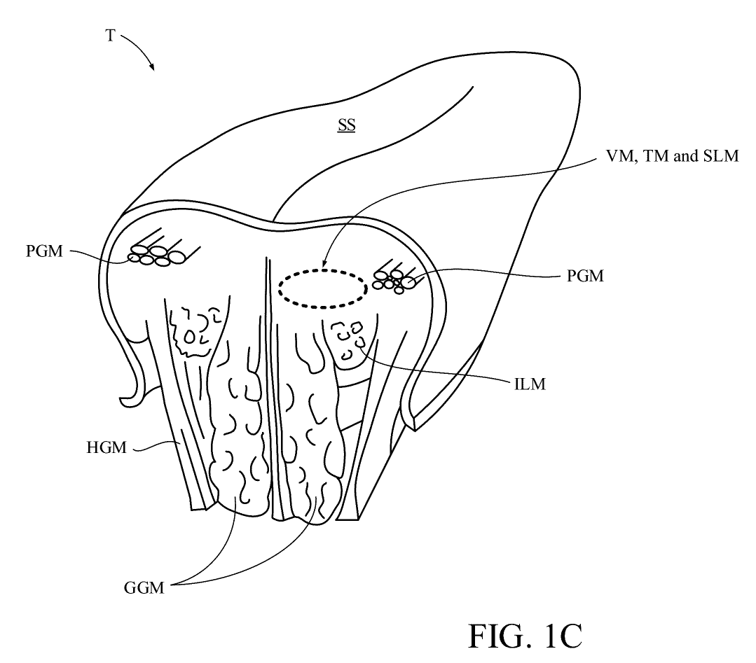

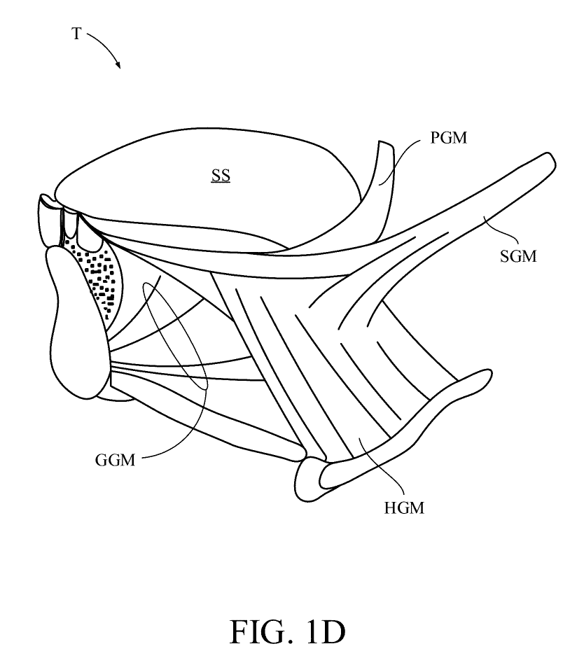

To more fully understand the present embodiments, the dynamics of OSA are first described with respect to an illustration 100 of a patient's oral cavity shown in FIGS. 1A-1D, which illustrate the anatomical elements of a patient's upper airway (e.g., including the nasal cavity, oral cavity, and pharynx of the patient). Referring first to FIGS. 1A-1B, the hard palate HP overlies the tongue T and forms the roof of the oral cavity OC (e.g., the mouth). The hard palate HP includes bone support BS, and thus does not typically deform during breathing. The soft palate SP, which is made of soft material such as membranes, fibrous material, fatty tissue, and muscle tissue, extends rearward (e.g., in a posterior direction) from the hard palate HP towards the back of the pharynx PHR. More specifically, an anterior end 1 of the soft palate SP is anchored to a posterior end of the hard palate HP, and a posterior end 2 of the soft palate SP is un-attached. Because the soft palate SP does not contain bone or hard cartilage, the soft palate SP is flexible and may collapse onto the back of the pharynx PHR and/or flap back and forth (e.g., especially during sleep).

The pharynx PHR, which passes air from the oral cavity OC and nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the Palatoglossal arch PGA, which runs downward on either side to the base of the tongue T.

Although not shown for simplicity, the pharynx PHR includes the nasopharynx, the oropharynx, and the laryngopharynx. The nasopharynx lies between an upper surface of the soft palate SP and the wall of the throat (i.e., superior to the oral cavity OC). The oropharynx lies behind the oral cavity OC, and extends from the uvula U to the level of the hyoid bone HB. The oropharynx opens anteriorly into the oral cavity OC. The lateral wall of the oropharynx consists of the palatine tonsil, and lies between the Palatoglossal arch PGA and the Palatopharyngeal arch. The anterior wall of the oropharynx consists of the base of the tongue T and the epiglottic vallecula. The superior wall of the oropharynx consists of the inferior surface of the soft palate SP and the uvula U. Because both food and air pass through the pharynx PHR, a flap of connective tissue called the epiglottis EP closes over the glottis (not shown for simplicity) when food is swallowed to prevent aspiration. The laryngopharynx is the part of the throat that connects to the esophagus ES, and lies inferior to the epiglottis EP.

Referring also to FIGS. 1C-1D, the tongue T includes a plurality of muscles that may be classified as either intrinsic muscles or extrinsic muscles. The intrinsic muscles, which lie entirely within the tongue T and are responsible for altering the shape of the tongue T (e.g., for talking and swallowing), include the superior longitudinal muscle SLM, the inferior longitudinal muscle ILM, the vertical muscle VM, and the transverse muscle TM. The superior longitudinal muscle SLM runs along the superior surface SS of the tongue T under the mucous membrane, and may be used to elevate, retract, and deviate the tip of the tongue T. The inferior longitudinal muscle ILM lines the sides of the tongue T, and is attached to the Styloglossus muscle SGM. The vertical muscle VM is located along the midline of the tongue T, and connects the superior and inferior longitudinal muscles together. The transverse muscle TM divides the tongue at the middle, and is attached to the mucous membranes that run along the sides of the tongue T.

The extrinsic muscles, which attach the tongue T to other structures and are responsible for re-positioning (e.g., moving) the tongue, include the Genioglossus muscle GGM, the Hyoglossus muscle HGM, the Styloglossus muscle SGM, and the Palatoglossus muscle PGM. The Genioglossus muscle GGM may be used to protrude the tongue T and to depress the center of the tongue T. The Hyoglossus muscle HGM may be used to depress the tongue T. The Styloglossus muscle SGM may be used to elevate and retract the tongue T. The Palatoglossus muscle PGM may be used to depress the soft palate SP and/or to elevate the back (posterior portion) of the tongue T. Referring also to FIGS. 1A and 1B, the Palatoglossus muscle PGM connects the tongue T to both sides of the Palatoglossus arch PGA, and inserts into lateral posterior regions 101 of the base of the tongue T.

It is noted that all of the muscles of the tongue T, except for the Palatoglossus muscle PGM, are innervated by the Hypoglossal nerve (not shown for simplicity); the Palatoglossus muscle PGM is innervated by the pharyngeal branch of the Vagus nerve (not shown for simplicity).

During awake periods, the muscles of the upper airway (as well as the hypoglossal nerve) are active and stimulated, and may maintain upper airway patency by preventing the soft palate SP from collapsing and/or by preventing the tongue T from prolapsing onto the back of the pharynx PHR. However, during sleep periods, a relative relaxed state of the soft palate SP may allow the soft palate SP to collapse and obstruct normal breathing, while a relative relaxed state of the tongue T may allow the tongue T to move in a posterior direction (e.g., onto the back of the pharynx PHR) and obstruct normal breathing.

Accordingly, conventional electrostimulation treatments for OSA typically involve causing the tongue T to move forward in the anterior direction during apnea episodes so that the tongue T does not collapse in the posterior direction. More specifically, some conventional techniques (e.g., disclosed in U.S. Pat. Nos. 5,190,053 and 6,212,435) electrically stimulate the Genioglossus muscle to move the tongue forward in an anterior direction during apnea episodes, while other conventional techniques (e.g., disclosed in U.S. Pat. No. 8,359,108) electrically stimulate the Hypoglossal nerve, which in turn causes the tongue to move forward in the anterior direction by innervating the Genioglossus muscle.

Unfortunately, repeatedly moving the tongue T forward (e.g., in the anterior direction) to prevent its prolapse into the back of the pharynx PHR may undesirably wake-up the patient, which defeats the very purpose of OSA treatments and may also abrade the tongue on the teeth. Indeed, electrically stimulating the relatively large Genioglossus muscle may cause discomfort or pain. In addition, because the Hypoglossal nerve innervates every tongue muscle except the Palatoglossus muscle PGM, electrically stimulating the Hypoglossal nerve may stimulate not only the Genioglossus muscle GGM but also the superior longitudinal muscle SLM, the inferior longitudinal muscle ILM, the vertical muscle VM, the transverse muscle TM, the Hyoglossus muscle HPM, and/or the Styloglossus muscle SSM. Stimulating multiple tongue muscles at the same time, in an attempt to move the tongue forward during apnea episodes, may not only over-stimulate the patient's tongue muscles but may also cause the tongue T to behave erratically (e.g., repeatedly protruding and retracting). For example, simultaneously stimulating the Genioglossus muscle GGM and the Styloglossus muscle SGM may cause the tongue T to repeatedly protrude and retract, respectively, which is likely to disturb the patient's sleep patterns or even wake-up the patient.

Applicant has discovered that OSA may be more effectively treated by targeting the Palatoglossus muscle PGM for electrical stimulation (e.g., rather than targeting the Genioglossus muscle GGM or the Hypoglossal nerve for electrical stimulation). More specifically, Applicant has discovered that application of one or more voltage differentials across selected portions of the patient's lateral or sublingual tissue may induce a current across the tongue to electrically stimulate the Palatoglossus muscle PGM in a manner that causes the Palatoglossus muscle PGM to shorten (e.g., to decrease its length). For at least some embodiments, the induced current may flow in a lateral direction across a base portion of the patient's tongue (e.g., proximate to the lateral points at which the Palatoglossus muscle inserts into the tongue T). Shortening the Palatoglossus muscle, using techniques described herein, may (1) stiffen and reduce the volume of the tongue T and (2) may cause the Palatoglossal arch PGA to pull down (e.g., in a downward direction) towards the base of the tongue T.

As described in more detail below, reducing the volume of the tongue T using techniques described herein may prevent the tongue T from prolapsing onto the back of the pharynx PHR, and pulling down the Palatoglossal arch PGA using techniques described herein may prevent the soft palate SP from collapsing onto the back of the pharynx PHR. In addition, stimulating the Palatoglossus muscle PGM using techniques described herein may also lower the superior surface SS of the tongue T, thereby causing the tongue to cinch downward (e.g., to “hunker down”) in a manner that further prevents obstruction of the patient's upper airway.

Perhaps equally important, because the present embodiments do not target either the Hypoglossal nerve or the Genioglossus muscle GGM for electrical stimulation, the present embodiments may not cause the tongue T to move forward in the anterior direction during application of the electrical stimulation, which in turn may reduce the likelihood of undesirably waking-up the patient. Indeed, for at least some embodiments, the voltage differential may be applied across the patient's sublingual or lateral lingual tissues in a manner that maintains the patient's tongue in a substantially stationary position while shortening the patient's Palatoglossus muscle PGM. In this manner, the present embodiments may maintain a patient's upper airway patency in a subtle yet therapeutic manner Although electrical stimulation of the Palatoglossus muscle PGM using techniques described herein is not intended to stimulate the Genioglossus muscle GGM, any inadvertent stimulation of the Genioglossus muscle GGM will be relatively small and, at most, may serve to maintain the tongue T in a substantially stationary position.

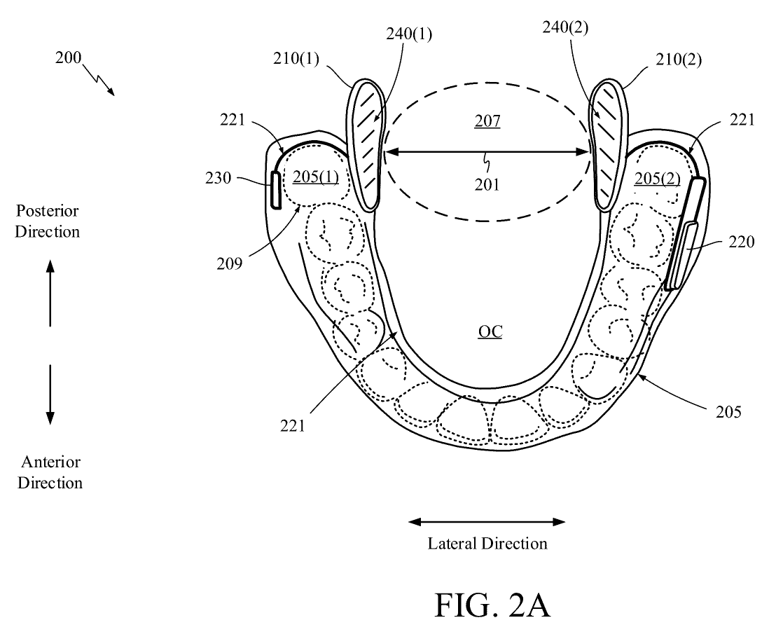

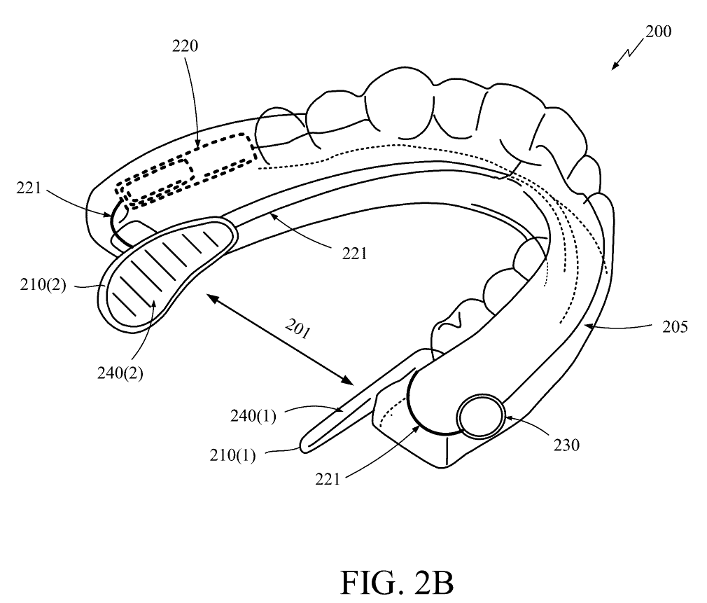

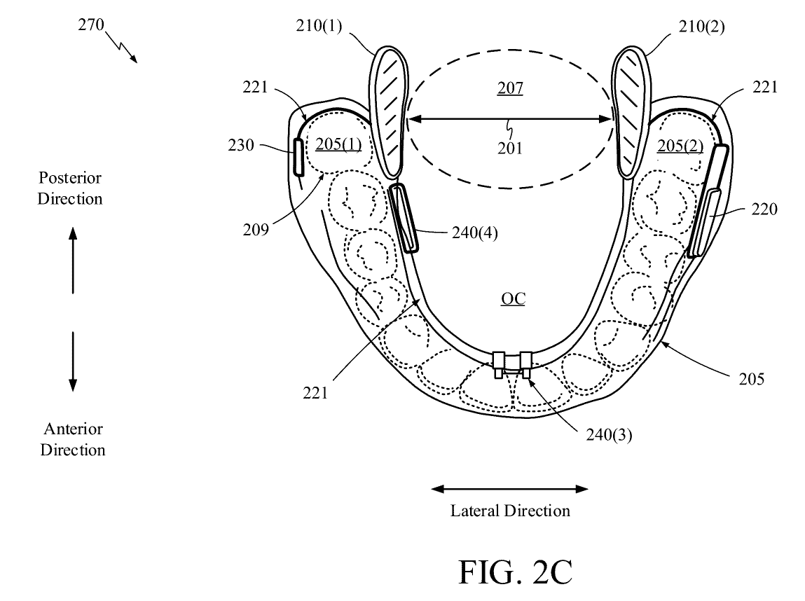

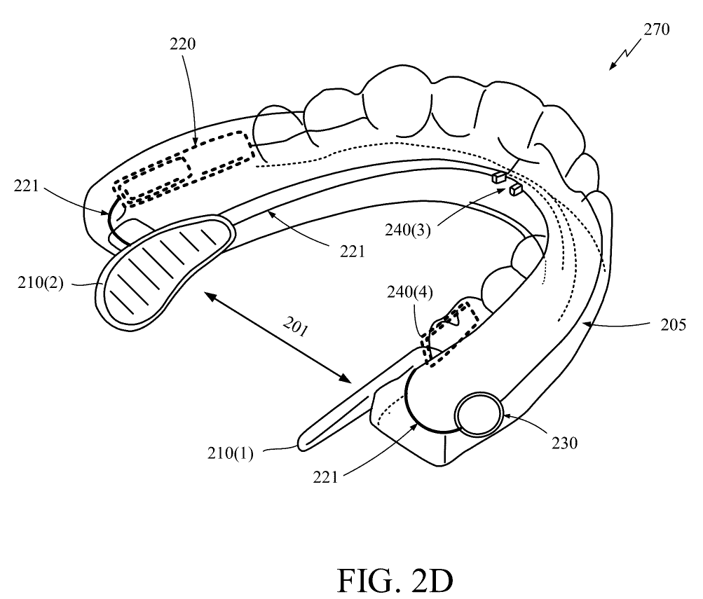

FIGS. 2A-2B show a removable oral appliance 200 that, in accordance with at least some embodiments, may be used to treat OSA by using electrical stimulation of the Palatoglossus muscle PGM to prevent collapse of the tongue T and soft palate SP into the back of the pharynx PHR. The appliance 200 is shown in FIGS. 2A-2B as including an appliance body 205 upon which a number of electrodes 210(1)-210(2), a control circuit 220, and a power supply 230 may be mounted (or otherwise attached to) so as to form a unitary and removable device that may fit generally within a patient's oral cavity OC (see also FIGS. 1A-1B). For such embodiments, there are no components external to the patient's body, and therefore the appliance 200 may not be associated with wires or other connectors that protrude from the patient's mouth or body. For some embodiments, the oral appliance 200 may be fitted over a patient's lower teeth and positioned to fit within a sublingual portion of the patient's oral cavity OC, for example, as depicted in FIG. 2A. For other embodiments, appliance 200 may be of other suitable configurations or structures, and the electrodes 210(1)-210(2) may be provided in other suitable positions. For some embodiments, there may be a minor portion of the oral appliance that protrudes slightly outside the lips or mouth. For other embodiments, the control circuit 220, power supply 230, and/or other components may be detached from the appliance 200 and located outside the patient's mouth. For such embodiments, the control circuit 220, power supply 230, and/or other components may be electrically coupled to the electrodes 210(1)-210(1) using wired connections (e.g., conductive wires).

Although only two electrodes 210(1)-210(1) are shown in FIGS. 2A-2B, it is to be understood that the appliance 200 may, in other embodiments, include a greater or fewer number of electrodes. For example, in other embodiments, the appliance 200 may include four or another number of electrodes 210 arranged in opposing (e.g., “X”) patterns with respect to the patient's sublingual tissues, wherein pairs of the electrodes may be selectively enabled and disabled in a manner that alternately induces two or more currents across the patient's sublingual tissues. For such other embodiments, each of such electrodes may be turned on and/or off independently of the other electrodes, for example, to determine a pair (or more) of electrodes that, at a particular moment for the patient, correlate to optimum electrical stimulation. The determined pair of electrodes may be dynamically selected either by (1) directly correlating electrical stimulation and immediate respiratory response or by (2) indirectly using the oral appliance 200 “to look for” the lowest impedance electrode “pair(s).” The determined electrodes may or may not be at the ends of an “X” pattern, and may be opposing one another.

The first electrode 210(1) and the second electrode 210(2), which may be formed using any suitable material and may be of any suitable size and/or shape, are connected to the control circuit 220 by wires 221. The wires 221 may be any suitable wire, cable, conductor, or other conductive element that facilitates the exchange of signals between control circuit 220 and the electrodes 210(1)-210(2). The control circuit 220 and electrodes 210(1)-210(2) are electrically coupled to power supply 230 via wires 221. Note that the wires 221 may be positioned either within or on an outside surface of the appliance body 205, and therefore do not protrude into or otherwise contact the patient's tongue or oral tissue. The power supply 230 may be mounted in any of several locations and may be any suitable power supply (e.g., a battery) that provides power to control circuit 220 and/or electrodes 210(1)-210(2). Bi-directional gating techniques may be used to control voltages and/or currents within wires 221, for example, so that wires 221 may alternately deliver power to electrodes 210(1)-210(2) and exchange electrical signals (e.g., sensor signals) between electrodes 240(1)-240(2) and control circuit 220.

For the example embodiment of FIGS. 2A-2B, the first electrode 210(1) may include or also function as a sensor 240(1), and the second electrode 210(2) may include or also function as a sensor 240(2), which could sense respiration or other functions of interest. In other words, for some embodiments, one or both of electrodes 210(1)-210(2) may also function as sensors such as respiration sensors. For such embodiments, the active function of the electrodes 210(1)-210(2) may be controlled using bi-directional gating techniques. For example, when the first electrode 210(1) is to function as a driven electrode, the bi-directional gating technique may connect the first electrode 210(1) to the output of a circuit such as a voltage and/or current driver (e.g., included within or associated with control circuit 220), for example, to provide a first voltage potential at the first electrode 210(1); conversely, when the first electrode 210(1) is to function as the respiration sensor or other sensor 240(1), the bi-directional gating technique may connect sensor 240(1) to the input of a circuit such as an amplifier and/or an ADC (analog to digital) converter (e.g., included within or associated with control circuit 220), for example, to sense a respiratory function of the patient. Similarly, when the second electrode 210(2) is to function as a driven electrode, the bi-directional gating technique may connect the second electrode 210(2) to the output of a circuit such as a voltage and/or current driver (e.g., included within or associated with control circuit 220), for example, to provide a second voltage potential at the second electrode 210(2); conversely, when the second electrode 210(2) is to function as the respiration sensor or other sensor 240(2), the bi-directional gating technique may connect sensor 240(2) to the input of a circuit such as an amplifier and/or an ADC (analog to digital) converter (e.g., included within or associated with control circuit 220), for example, to sense a respiratory function of the patient.

The respiration sensors or other sensors 240(1)-240(2), as provided within or otherwise associated with the electrodes 210(1)-210(2), may be any suitable sensors that measure any physical, chemical, mechanical, electrical, neurological, and/or other characteristics of the patient which may indicate or identify the presence and/or absence of disturbed breathing. These respiration sensors 240(1)-240(2) may also be used to detect snoring. For at least some embodiments, one or both of electrodes 210(1)-210(2) may include electromyogram (EMG) sensor electrodes that, for example, detect electrical activity of the muscles and/or nerves within, connected to, or otherwise associated with the oral cavity. For at least one embodiment, one or both of electrodes 210(1)-210(2) may include a microphone (or any other sensor to sense acoustic and/or vibration energy) to detect the patient's respiratory behavior. For other embodiments, one or both of electrodes 210(1)-210(2) may include one or more of the following non-exhaustive list of sensors: accelerometers, piezos, capacitance proximity detectors, capacitive sensing elements, optical systems, EMG sensors, etc.

For other embodiments, electrodes 210(1)-210(2) may not include any sensors. For at least one of the other embodiments, the electrodes 210(1)-210(2) may continuously provide electrical stimulation to the patient's Palatoglossus muscle PGM via the lingual tissues. For an alternative embodiment, a timer (not shown for simplicity) may be provided on appliance body 205 or within control circuit 220 and configured to selectively enable/disable electrodes 210(1)-210(2), for example, based upon a predetermined stimulation schedule. In another closed-loop embodiment, the electrodes 210(1)-210(2) may be selectively enabled/disabled based upon one or more sources of sensor feedback from the patient.

For the example embodiment of FIGS. 2A-2B, the first and second electrodes 210(1)-210(2) may be mounted on respective lateral arms 205(1) and 205(2) of the body 205 of appliance 200 such that when appliance 200 is placed within a sublingual portion of the patient's oral cavity OC, the first and second electrodes 210(1)-210(2) are positioned on opposite sides of the posterior sublingual region 207 of the patient's oral cavity OC. For other embodiments, the first and second electrodes 210(1)-210(2) may be separate from appliance body 205 but connected to respective lateral arms 205(1)-205(2), for example, so as to “float” beneath or on either side of the patient's tongue T, or alternatively oriented so as to be positioned on opposite sides of the superior surface of the tongue T. For some embodiments, the first and second electrodes 210(1)-210(2) are positioned in the posterior sublingual region 207 of the oral cavity OC such that at least a portion of each of the first and second electrodes 210(1)-210(2) is proximal to a molar 209 of the patient. In this manner, the first and second electrodes 210(1)-210(2) may be in physical contact with the patient's lingual tissues proximate to the lateral posterior regions (e.g., points) 101 at which the Palatoglossus muscle PGM inserts into the tongue T (see also FIGS. 1A-1B). Further, as depicted in FIGS. 2A-2B, the first and second electrodes 210(1)-210(2) may be angularly oriented with respect to the floor of the mouth such that the first and second electrodes 210(1)-210(2) substantially face and/or contact opposite sides of the tongue T proximate to the lateral posterior regions (e.g., points) 101 at which the Palatoglossus muscle PGM inserts into the tongue T (see also FIGS. 1A-1B). For other embodiments, the first and second electrodes 210(1)-210(2) may be provided in one or more other positions and/or orientations.

The control circuit 220 may provide one or more signals to the first and second electrodes 210(1)-210(2) to create a voltage differential across the patient's lingual tissues (e.g., across the base of the tongue) in the lateral direction. For purposes of discussion herein, the first electrode 210(1) may provide a first voltage potential V1, and the second electrode 210(2) may provide a second voltage potential V2. The voltage differential (e.g., V2-V1) provided between the first and second electrodes 210(1)-210(2) may induce a current 201 in a substantially lateral direction across the patient's lingual tissues. For some embodiments, the current 201 is induced in a substantially lateral direction across the patient's tongue. The current 201, which for some embodiments may be a reversible current (as described in more detail below), electrically stimulates the patient's Palatoglossus muscle PGM in a manner that shortens the Palatoglossus muscle PGM.

When the Palatoglossus muscle PGM is stimulated and/or shortened in response to the current 201 induced by the first and second electrodes 210(1)-210(2), the Palatoglossus muscle PGM causes the tongue T to stiffen in a manner that decreases the tongue's volume, and that may also slightly cinch a portion of the tongue T closer to the floor of the oral cavity OC. One or more of decreasing the tongue's volume and slightly cinching the tongue T downward towards the floor of the oral cavity OC may prevent the tongue T from prolapsing onto the back of the pharynx PHR, thereby maintaining patency of the patient's upper airway (e.g., without moving the tongue forward in the anterior direction). The shortening of the Palatoglossus muscle PGM may also pull the patient's Palatoglossal arch PGA in a downward direction towards the base of the tongue T, which in turn may prevent the soft palate SP from collapsing and obstructing the patient's upper airway.

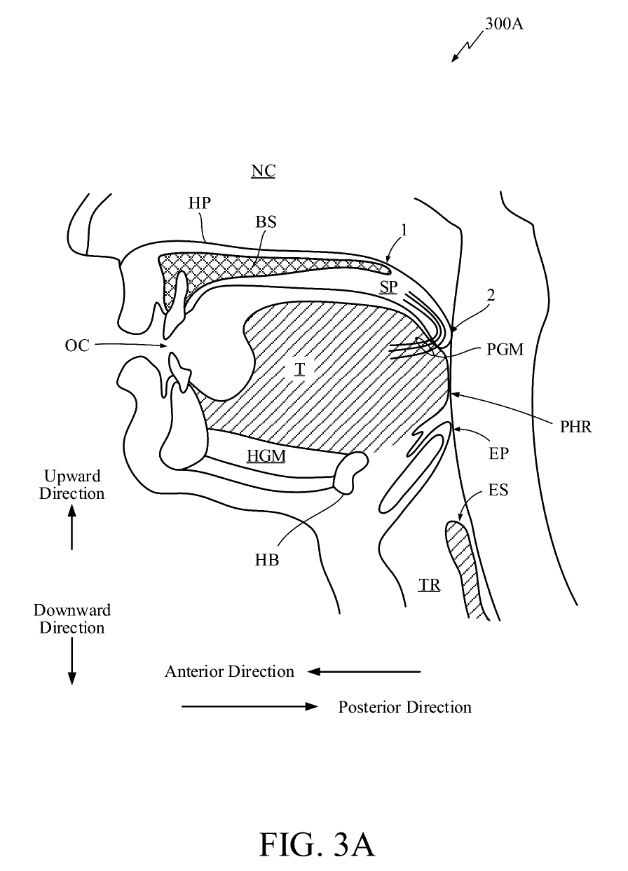

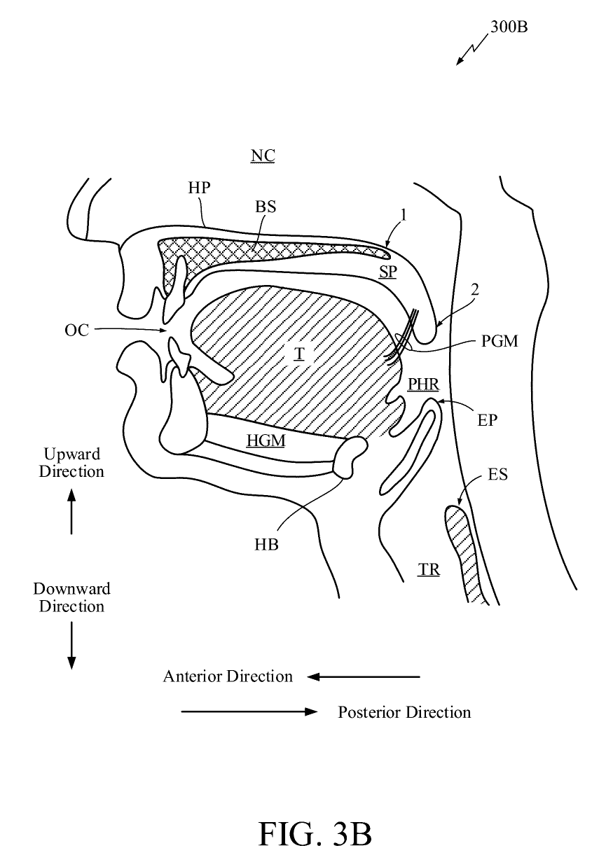

For example, FIG. 3A shows a side view 300A of a patient depicting the collapse of the patient's tongue T and soft palate SP in a posterior direction towards the back of the pharynx (PHR) during disturbed breathing. As depicted in FIG. 3A, the patient's upper airway is obstructed by the tongue T prolapsing onto the back wall of the pharynx PHR and/or by the soft palate SP collapsing onto the back wall of the pharynx PHR.

In contrast, FIG. 3B shows a side view 300B of the patient depicting the patient's upper airway response to electrical stimulation provided in accordance with the present embodiments. More specifically, electrical stimulation provided by one or more embodiments of the appliance 200 may cause the Palatoglossus muscle PGM to stiffen and shorten, which in turn may pull the patient's soft palate SP and/or palatal arches in a downward direction, thereby preventing the soft palate SP from collapsing onto the back wall of the pharynx PHR. In addition, stiffening and/or shortening the Palatoglossus muscle PGM may also cause the patient's tongue T to contract and/or cinch downward in a manner that prevents collapse of the tongue T towards the back of the pharynx PHR without substantially moving the tongue T forward in the anterior direction.

The control circuit 220 may be any suitable circuit or device (e.g., a processor) that causes electrical stimulation energy to be provided to areas proximate to the base of the patient's tongue T via the electrodes 210(1)-210(2). More specifically, the control circuit 220 may generate one or more voltage waveforms that, when provided as signals and/or drive signals to the first and second electrodes 210(1)-210(2), primarily induces a current across (e.g., in a substantially lateral direction) one or more portions of the patient's upper airway (e.g., across a lingual portion of the patient's tongue T) in a manner that causes the patient's Palatoglossus muscle PGM to shorten. As used herein, inducing a current across one or more portions of the patient's upper airway refers to a direction between left and right sides of the patient's oral cavity. The waveforms provided by control circuit 220 may include continuous voltage waveforms, a series of pulses, or a combination of both. The control circuit 220 may be formed using digital components, analog components, or a combination of analog and digital components.

For some embodiments, the control circuit 220 may vary or modify the waveform in a manner that induces a reversible current across one or more portions of the patient's upper airway (e.g., across a portion of the patient's tongue T). Applicant has discovered that inducing a reversible current across one or more portions of the patient's upper airway may decrease the likelihood of patient discomfort (e.g., as compared with providing a constant current or current in a single direction). More specifically, Applicant notes that when a current is induced in the lingual tissues of the patient, the lingual tissues may experience ion or carrier depletion, which in turn may require greater voltage differentials and/or greater current magnitudes to maintain a desired level of electrical stimulation of the Palatoglossus muscle PGM. However, inducing greater voltage and/or current magnitudes to offset increasing levels of ion or carrier depletion may create patient discomfort. Thus, to prevent ion or carrier depletion of the patient's sublingual tissues, the control circuit 220 may limit the duration of pulses that induce the current 201 across the sublingual tissues and/or may from time to time reverse the direction (e.g., polarity) of the current 201 induced across the patient's sublingual tissues.

For some embodiments, control circuit 220 may generate and/or dynamically adjust the waveform and/or drive waveform provided to the first and second electrodes 210(1)-210(2) (and/or to a number of additional electrodes, not shown for simplicity) in response to one or more input signals indicative of the patient's respiratory behavior and/or inputs from other characteristics and sensing methods. The input signals may be provided by one or more of the sensors 240(1)-240(2) integrated within respective electrodes 210(1)-210(2).

For other embodiments, sensors other than the sensors 240(1)-240(2) integrated within respective electrodes 210(1)-210(2) may be used to generate the input signals. For example, FIGS. 2C-2D show a removable oral appliance 270 in accordance with other embodiments. Appliance 270 may include all the elements of the appliance 200 of FIGS. 2A-2B, plus additional sensors 240(3)-240(4). For the example embodiment of FIGS. 2C-2D, the sensor 240(3) may be an oxygen saturation (O₂ sat) sensor that provides a signal indicative of the patient's oxygen saturation level, and the sensor 240(4) may be a vibration sensor that provides a signal indicative of the patient's respiratory activity (as measured by vibrations detected within the patient's oral cavity). For other embodiments, sensors 240(3)-240(4) may be other types of sensors including, for example, sensors that measure air composition (especially O₂ and CO₂), heart rate, respiration, temperature, head position, snoring, pH levels, and others.

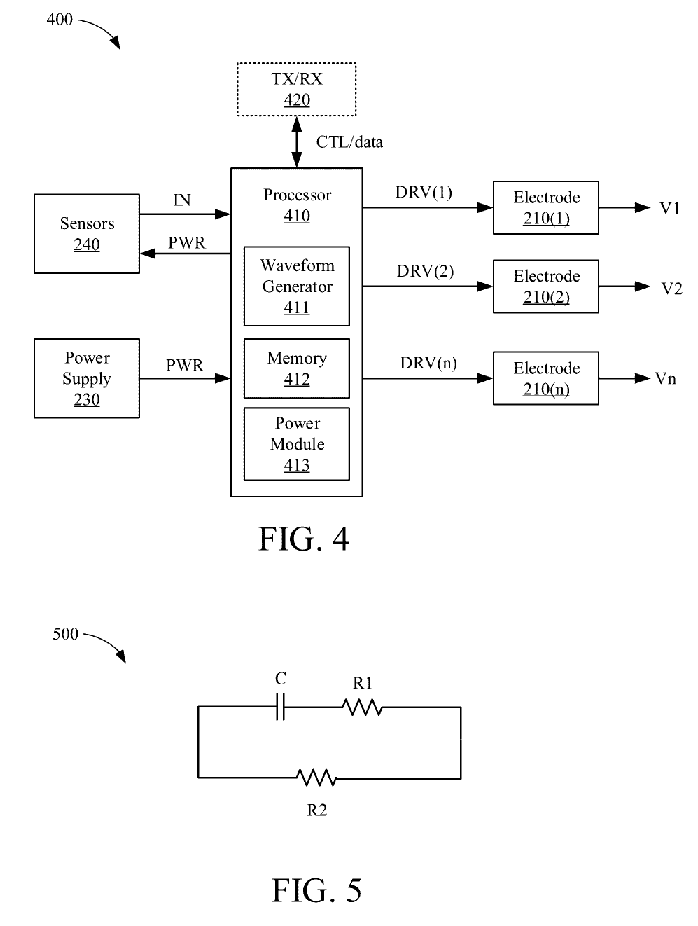

FIG. 4 shows a block diagram of the electrical components of an appliance 400 that is one embodiment of the appliance 200 of FIGS. 2A-2B. Appliance 400 is shown to include a processor 410, a plurality of electrodes 210(1)-210(n), power supply 230, sensors 240, and an optional transceiver 420. Processor 410, which is one embodiment of the control circuit 220 of FIGS. 2A-2B, includes a waveform generator 411, a memory 412, and a power module 413. The power supply 230, which as mentioned above may be any suitable power supply (e.g., a battery), provides power (PWR) to processor 410. For some embodiments, the processor 410 may use power module 413 to selectively provide power to sensors 240, for example, only during periods of time that the sensors 240 are to be active (e.g., only when it is desired to receive input signals from sensors 240). Selectively providing power to sensors 240 may not only reduce power consumption (thereby prolonging the battery life of power supply 230) but may also minimize electrical signals transmitted along wires 221 to the processor 410. For other embodiments, power supply 230 may provide power directly to sensors 240.

The sensors 240, which may include sensors 240(1)-240(2) of FIGS. 2A-2B and/or sensors 240(3)-240(4) of FIGS. 2C-2D, may provide input signals to processor 410. The input signals may be indicative of the respiratory behavior or other functions of the patient and may be used to detect the presence and/or absence of disturbed breathing, for example, as described above with respect to FIGS. 2A-2D.

The processor 410 may receive one or more input signals from sensors 240, or sensors located elsewhere, and in response thereto may provide signals and/or drive signals (DRV) to a number of the electrodes 210(1)-210(n). As described above, the signals and/or drive signals (e.g., voltage and/or current waveforms) generated by waveform generator 411 may cause one or more of the electrodes 210(1)-210(n) to electrically stimulate one or more portions of the patient's oral cavity OC in a manner that shortens the patient's Palatoglossus muscle PGM. Shortening the Palatoglossus muscle PGM in response to electrical stimulation provided by one or more of the electrodes 210(1)-210(n) may (1) stiffen and reduce the volume of the tongue T, (2) may cause the tongue to cinch downward, and (3) may cause the Palatoglossal arch PGA to pull down (e.g., in a downward direction) towards the base of the tongue T. In this manner, the electrical stimulation provided by the one or more electrodes 210(1)-210(n) may prevent the tongue T from prolapsing onto the back of the pharynx PHR and/or may prevent the soft palate SP from collapsing onto the back of the pharynx PHR and/or may prevent the tissues from vibrating.

As mentioned above, the waveforms generated by the waveform generator 411, when provided as signals and/or drive signals to the electrodes 210(1)-210(n), primarily induce a current across the patient's upper airway in a manner that causes the patient's Palatoglossus muscle PGM to shorten. The waveforms generated by the waveform generator 411 may include continuous (analog) voltage waveforms, any number of pulses that may vary in shape and duration as a pulse train, or the pulses may be combined to simulate an analog waveform or a combination of both, and may be dynamically modified by the waveform generator 411. In other implementations, the waveforms generated by the waveform generator 411 may be digital pulses.

The optional transceiver 420 may be used to transmit control information (CTL) and/or data, and/or receive control information and/or data from an external device via a suitable wired or wireless connection. The external device (not shown for simplicity) may be any suitable display device, storage device, distribution system, transmission system, and the like. For one example, the external device may be a display (e.g., to display the patient's respiratory behavior or patterns, to alert an observer to periods of electrical stimulation, to indicate an alarm if breathing stops, and so on).

For another example, the external device may be a storage device that stores any data produced by appliance 200, perhaps including the patient's respiratory behavior, the electrical stimulation provided by appliance 200, the waveforms provided by waveform generator 411, and/or relationships between two or more of the above. More specifically, for some embodiments, the external device may store data for a plurality of patients indicating, for example, a relationship between the application of electrical stimulation to the patient and the patient's respiratory response to such electrical stimulation, and may include other information. Such relationship data for large numbers of patients may be aggregated, and thereafter used to identify trends or common components of OSA across various population demographics. The storage device may be a local storage device, or may be a remote storage device (e.g., accessible via one or more means and/or networks including but not limited to such as a wide area network (WAN), a wireless local area network (WLAN), a virtual private network (VPN), and/or the Internet). The data and information may be made available and/or manipulated locally and/or remotely, and may be utilized immediately and/or preserved for later utilization and/or manipulation.

Memory 412 may include a non-transitory computer-readable storage medium (e.g., one or more nonvolatile memory elements, such as EPROM, EEPROM, Flash memory, a hard drive, etc.) that may store the following software modules and/or information:
a function select module to selectively switch an active function of the electrodes 210 between an electrode mode (e.g., provided by one or more of electrodes 210 and a sensor mode (e.g., provided by one or more of sensors 240);
a control module to selectively provide signals and/or drive signals to the electrodes 210, for example, to induce an electric current across a portion of the patient's oral cavity in accordance with the present embodiments and/or to receive input signals from the sensors 240; and
a data collection module to record data indicative of the patient's respiratory or other behavior and/or to transmit such data to an external device.

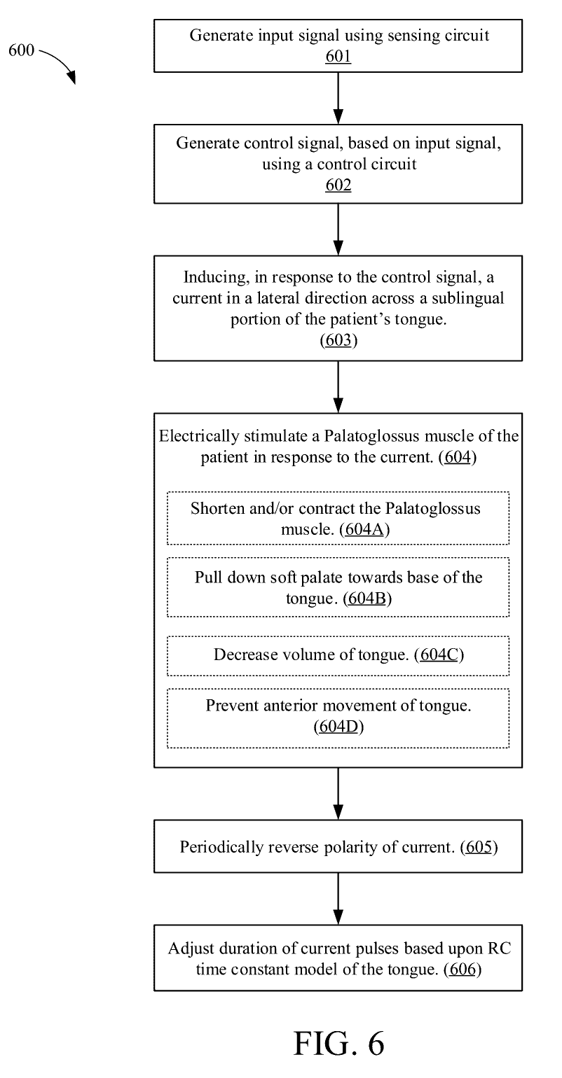

Each software module may include instructions that, when executed by the processor 410, may cause appliance 400 to perform the corresponding function. Thus, the non-transitory computer-readable storage medium of memory 412 may include instructions for performing all or a portion of the operations described below with respect to FIG. 6. The processor 410 may be any suitable processor capable of executing scripts of instructions of one or more software programs stored in the appliance 400 (e.g., within memory 412). For at least some embodiments, memory 412 may include or be associated with a suitable volatile memory, for example, to store data corresponding to the patient's respiratory functions and/or corresponding to the electrical stimulation provided by the appliance 200.

As mentioned above, the control circuit 220 may control the duration of pulses that induce the current 201 across the patient's oral cavity, for example, to minimize carrier depletion within the patient's lingual tissues and/or may from time to time reverse the direction of the induced current 201, for example, to provide a zero sum drive waveform (e.g., to minimize or preclude electrochemical activity and/or to minimize the patient's awareness of any electrical activity related to oral appliance 200). For at least one embodiment, the control circuit 220 may select the pulse lengths (and/or other characteristics of the waveforms) based upon a resistive-capacitive (RC) time constant model of the patient's tongue T. For example, FIG. 5 shows an RC time constant model 500 of the patient's tongue T. The model 500 is shown to include a capacitor C and two resistors, R1 and R2. For an example embodiment, the capacitor C may be approximately 0.5uF, the resistor R1 may be approximately 600 ohms, and the resistor R2 may be approximately 4,000 ohms. Thus, for the example embodiment, the time constant τ=R1*C may be a value approximately equal to 300 μs. The resistor R2 represents minor “DC current” flow in the model, where the current stabilizes at a small but non-zero value after more than 5 time constants or when DC is applied to the electrodes.

More specifically, Applicant has discovered that a typical patient's tongue T is often most receptive to a current “pulse duration” that is equal to or shorter than a time period approximately equal to τ=R1*C≈300 μs. After the time period 3τ≈1 ms expires, the patient's tongue T may exhibit an even greater increase in impedance, or perhaps experience ion depletion, which in turn requires greater voltage levels to continue inducing the current 201 across the patient's upper airway tissues. As noted above, increasing the voltage levels to continue inducing the current 201 across the patient's upper airway tissues may not only waste battery or wired power but also may cause discomfort (or even pain) to the patient. Indeed, because current regulators typically utilize their available voltage “headroom” to increase the drive voltage and maintain a constant current flow when the load impedance increases or when the effective drive voltage otherwise decreases, it is important to dynamically manage the effective drive voltage provided by the electrodes 210(1)-210(2).

The effective drive voltage may decrease when there is an increased impedance, or perhaps ion depletion, in the patient's tongue, and the drive resistance may increase when one (or both) of the electrodes 210(1)-210(2) loses contact with the patient's sublingual tissues, generally causing the control circuit 220 to increase its drive voltage in an attempt to maintain a prescribed current flow. Thus, for at least some embodiments, the control circuit 220 may be configured to limit the drive voltage and/or the current to levels that are known to be safe and comfortable for the patient, even if the drive impedance becomes unusually high. In addition, the control circuit 220 may be configured to from time to time reverse the polarity or direction of the induced current 201. The reversal of the current 201 can be performed at any time. The timing of the reversal of current 201 may be selected such that there is no net transfer of charge across the patient's sublingual tissues (e.g., a zero sum waveform).

FIG. 6 is a flow chart 600 depicting an example operation for providing electrical stimulation to a patient in accordance with the present embodiments. Although the flow chart 600 is discussed below with respect to appliance 200 of FIGS. 2A-2B, the flow chart 600 is equally applicable to other embodiments discussed herein. Prior to operation, the appliance 200 is positioned within a sublingual portion of the patient's oral cavity, for example, so that the electrodes 210(1)-210(2) are positioned on opposite sides of the patient's tongue proximate to the lateral posterior regions (e.g., points) 101 at which the Palatoglossus muscle PGM inserts into the tongue T (see also FIGS. 1A-1B). Once the appliance 200 is properly fitted within the patient's oral cavity, the appliance 200 accepts zero or more input signals using a number of sensing circuits provided on or otherwise associated with appliance 200 (601). As discussed above, the input signals may be indicative of the respiratory state or other behavior of the patient, and may be derived from or generated by any suitable sensor. The control circuit 220 generates a number of control and/or drive signals based on the input signals. (602).

In response to the signals and/or drive signals, the electrodes 210(1)-210(2) induce a current in a lateral direction across a sublingual portion of the patient's tongue (603). The current induced across the sublingual portion of the patient's tongue electrically stimulates the patient's Palatoglossus muscle (604). As described above, electrically stimulating the patient's Palatoglossus muscle may shorten the Palatoglossus muscle (604A), may pull down the patient's soft palate towards the base of the tongue (604B), may decrease the volume of the tongue (604C), and/or may prevent anterior movement of the tongue (604D).

For some embodiments, the induced current may be a reversible current. For at least one embodiment, the reversible current may be a zero-sum waveform. For such embodiments, the control circuit 220 may from time to time reverse a polarity of the reversible current (605), and/or may adjust the duration and/or amplitude of voltage and/or current pulses and/or waveforms based on the RC time constant model of the patient's tongue (606).

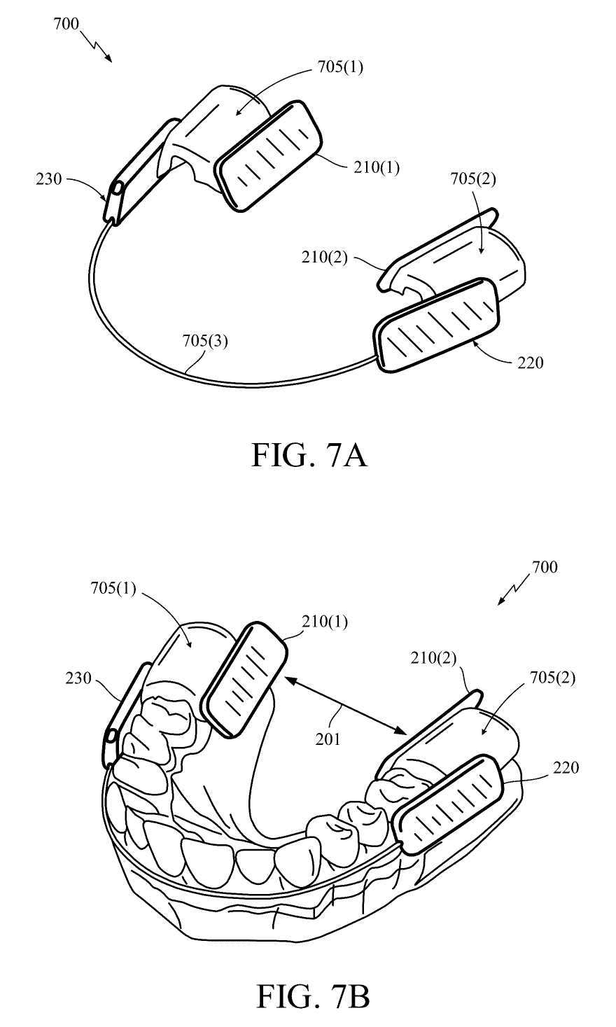

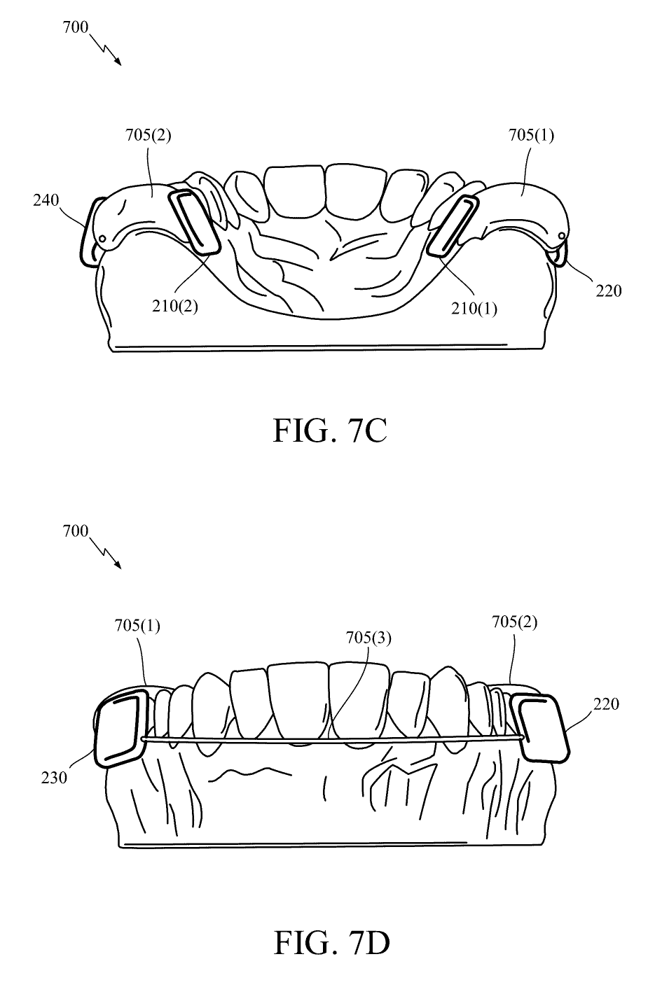

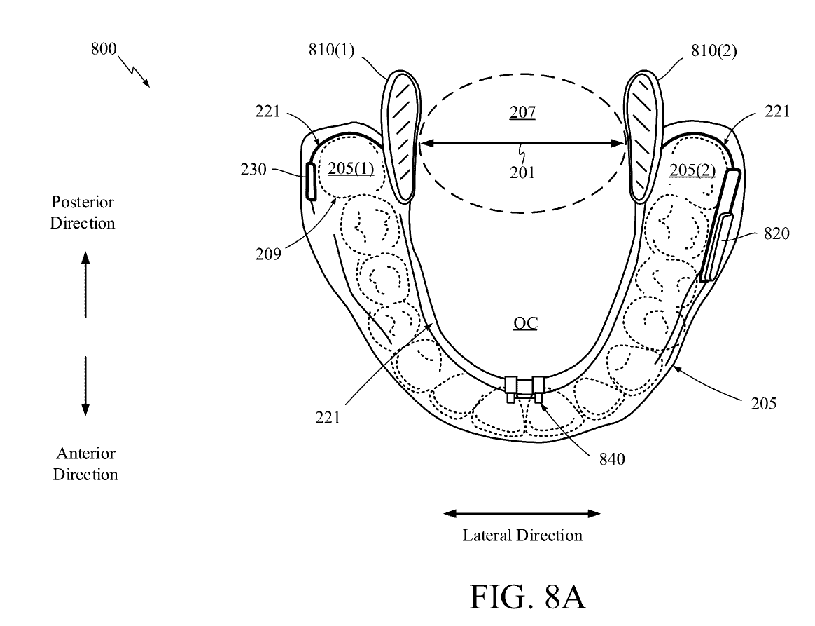

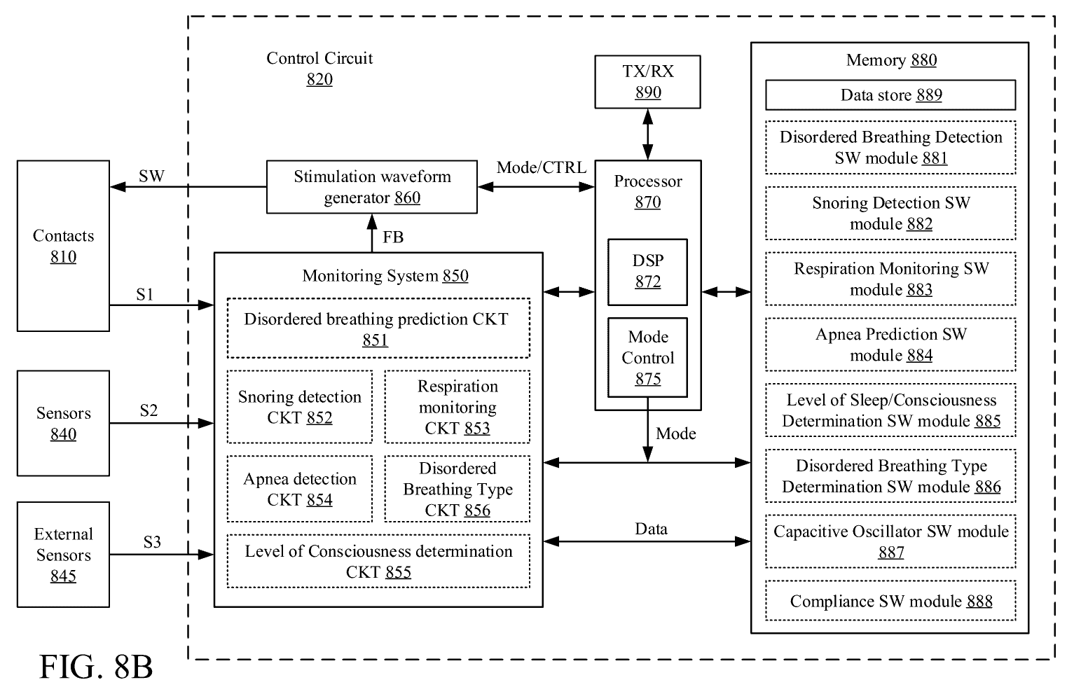

FIGS. 7A-7D show a removable oral appliance 700 in accordance with other embodiments. The oral appliance 700, which may be used to treat OSA (and/or other types of disordered breathing, discussed in more detail below with respect to FIGS. 8A-8B, 9A-9E, 10A-10D, and 11A-11E) by providing electrical stimulation to a patient's sublingual tissues in a manner that causes the Palatoglossus muscle to shorten, is shown to include an appliance body 705 (which includes portions 705(1)-705(3), as shown in the FIGS.) upon which electrodes 210(1)-210(2), control circuit 220, and power supply 230 may be mounted (or otherwise attached to) so as to form a unitary and removable device that may fit entirely within a patient's oral cavity OC (see also FIGS. 1A-1B). The oral appliance 700, which may operate in a similar manner as the oral appliance 200 of FIGS. 2A-2B, includes appliance body 705 instead of appliance body 205 of FIGS. 2A-2B. Specifically, appliance body 705 includes two anchor portions 705(1)-705(2) and a support wire 705(3). The anchor portions 705(1)-705(2) may be fitted over opposite or approximately opposite molars of the patient, with the support wire 705(3) connected between anchor portions 705(1)-705(2) and extending along the patient's gum line. For other embodiments, the appliance body 705 may be attached, inserted, or otherwise positioned within the patient's oral cavity in any technically feasible manner.

More specifically, for the example embodiments described herein, the first electrode 210(1) may be attached to or otherwise associated with the first anchor portion 705(1), and the second electrode 210(2) may be attached to or otherwise associated with the second anchor portion 705(2). For other embodiments, one or both of the anchor portions 705(1)-705(2) may be omitted (e.g., the appliance body 705 may be a “floating” system in which the electrodes 210(1)-210(2) are positioned within the patient's oral cavity without anchors that fit over the patient's teeth. The control circuit 220 may be attached to support wire 705(3) and/or the second anchor portion 705(2), and the power supply 230 may be attached to support wire 705(3) and/or the first anchor portion 705(1) and/or the second anchor portion 705(2). Wires 221 (not shown in FIGS. 7A-7D for simplicity) may be attached to or provided within the support wire 705(3).

As discussed above with respect to FIGS. 2A-2D and 7A-7D, the sensors 240 of the example embodiments may be contacts (e.g., EMG surface electrodes) that detect electrical activity of a patient's upper airway musculature and/or nerves, and in response thereto may generate one or more electrical signals indicative of such electrical activity. The electrical signals may be used to initiate, adjust, and/or terminate the electrical stimulation provided to the patient's oral cavity by the induced current 201 across one or more portions of the patient's oral cavity. Thus, electrical activity detected by sensors 240 may be used to control the timing, frequency, duration, and/or magnitude of the electrical stimulation provided by the example embodiments to maintain a patient's upper airway patency, as described above.

EMG is typically used to detect nerve dysfunction, muscle dysfunction, and problems with nerve-to-muscle signal transmission, for example, to identify neuromuscular diseases and disorders of motor control. There are two types of conventional EMG: surface EMG and intramuscular EMG. Surface EMG assesses muscle function by recording muscle activity using electrodes placed on the surface of the skin. Although non-invasive, surface EMG has limited applications and accuracy because electrical signals of the target muscles must travel through multiple layers of skin and fat tissue to reach the surface electrodes, and therefore may be weak and difficult to detect by the surface electrodes. In addition, the depth and density of the subcutaneous tissue between the surface electrodes and the target muscles may vary significantly between patients, which in turn may reduce accuracy of surface EMG recordings. As a result, intramuscular EMG is typically used for applications that require accurate recordings of a person's muscular activity.

Intramuscular EMG involves inserting a needle electrode directly into the target muscle of the patient, for example, so that the electrical signals of the target muscles can be measured without having to travel through subcutaneous tissue. To perform intramuscular EMG, the needle electrode is inserted through the skin into the muscle tissue, and then moved to multiple spots within a relaxed muscle to evaluate both insertional activity and resting activity in the muscle. Although intramuscular EMG may provide more accurate recordings of a muscle's electrical activity than surface EMG, intramuscular EMG is an invasive technique that not only causes significant patient discomfort but also requires a medical professional to administer. In addition, because needle electrodes have a relatively small surface area (e.g., on the order of 0.3 mm²) to allow for insertion through the patient's subcutaneous tissue and muscle fibers, the needle electrodes typically detect electrical activity of a relatively small portion of the target muscle.

Thus, neither conventional surface EMG techniques nor conventional intramuscular techniques are well-suited for non-invasively monitoring a patient's respiratory state or activity. Accordingly, there is a need to non-invasively monitor a patient's upper airway with a level of accuracy sufficient to predict the onset of disordered breathing in the patient, to detect a presence of disordered breathing in the patient, and to determine a type of the disordered breathing in the patient. These are at least some of the technical problems to be solved by the example embodiments described herein.

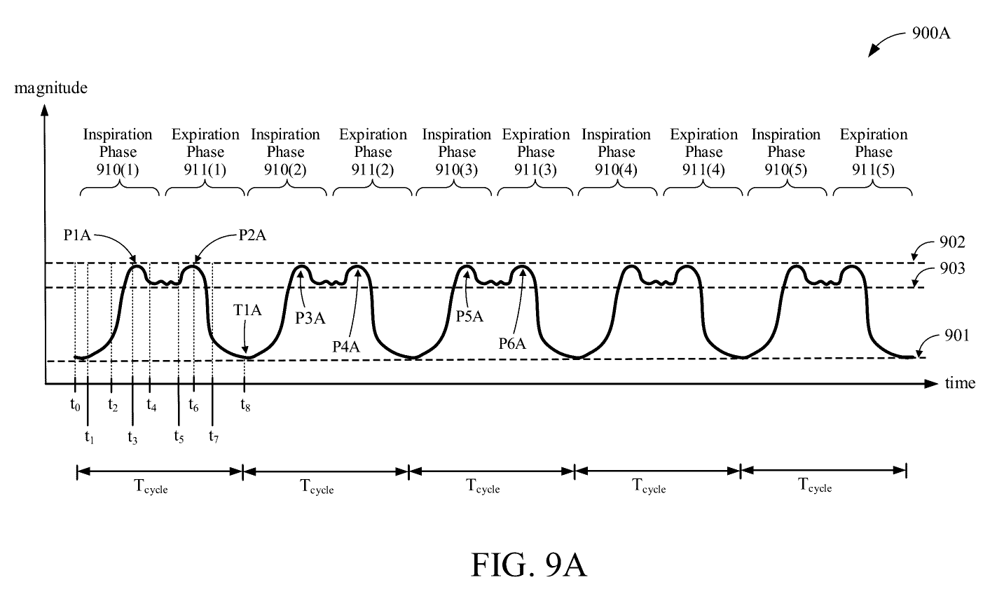

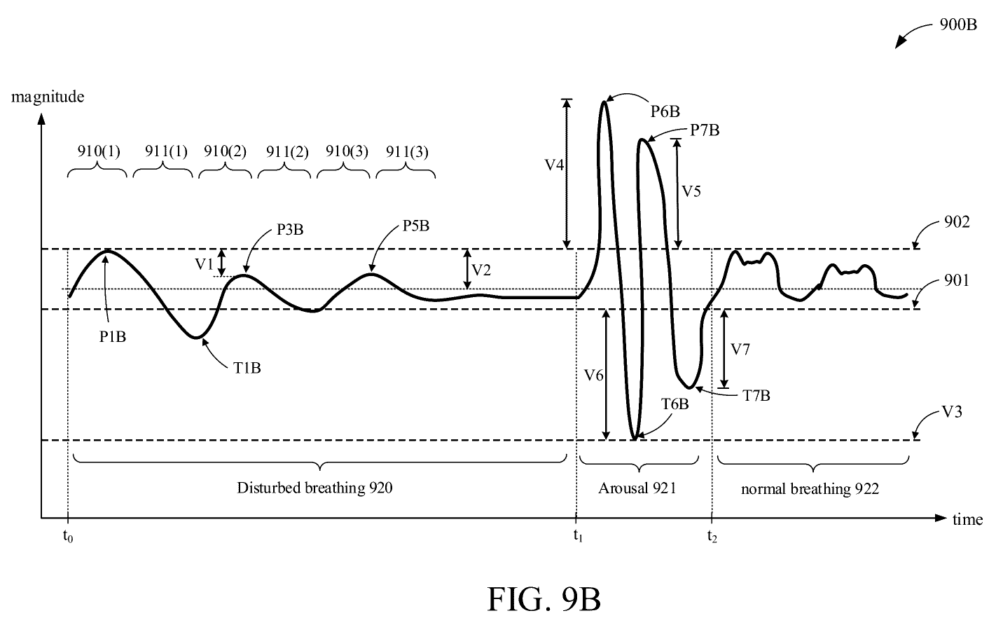

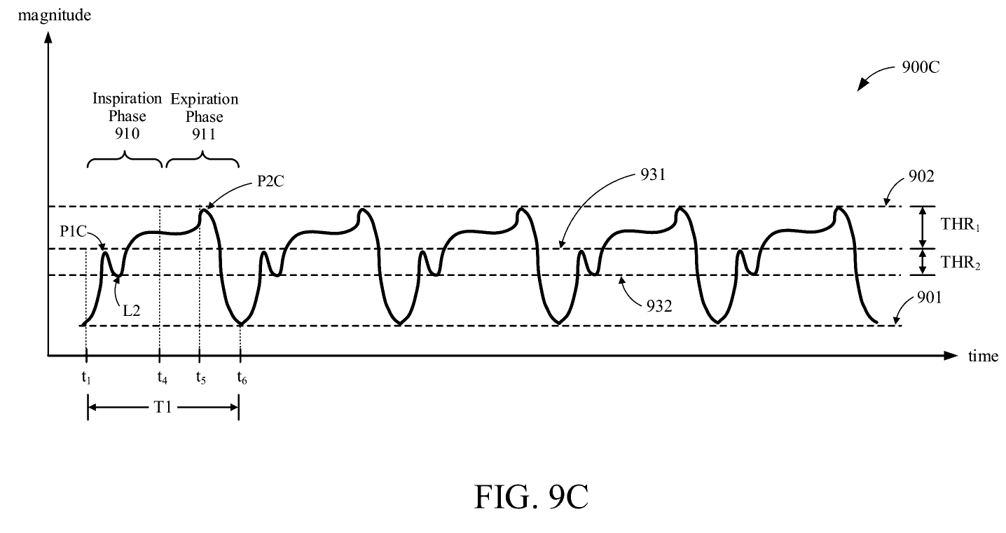

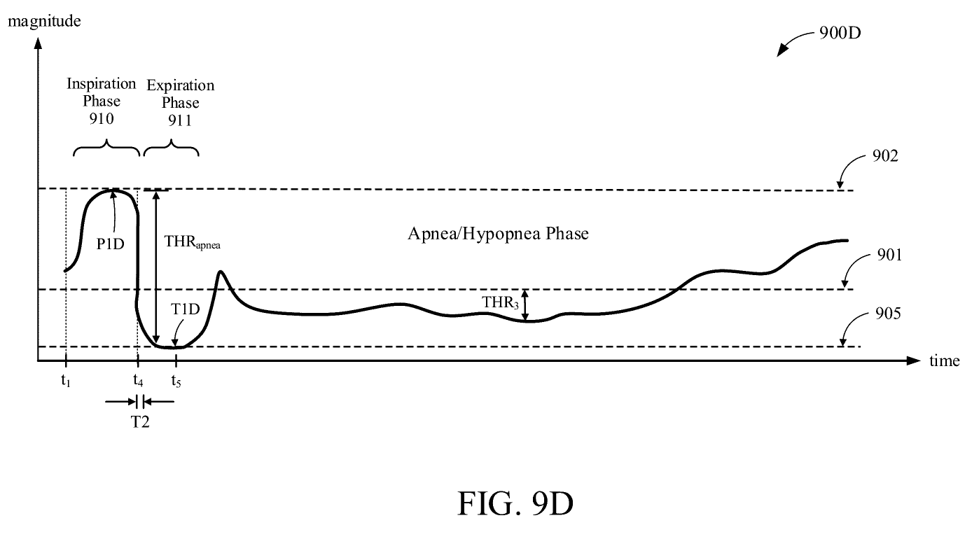

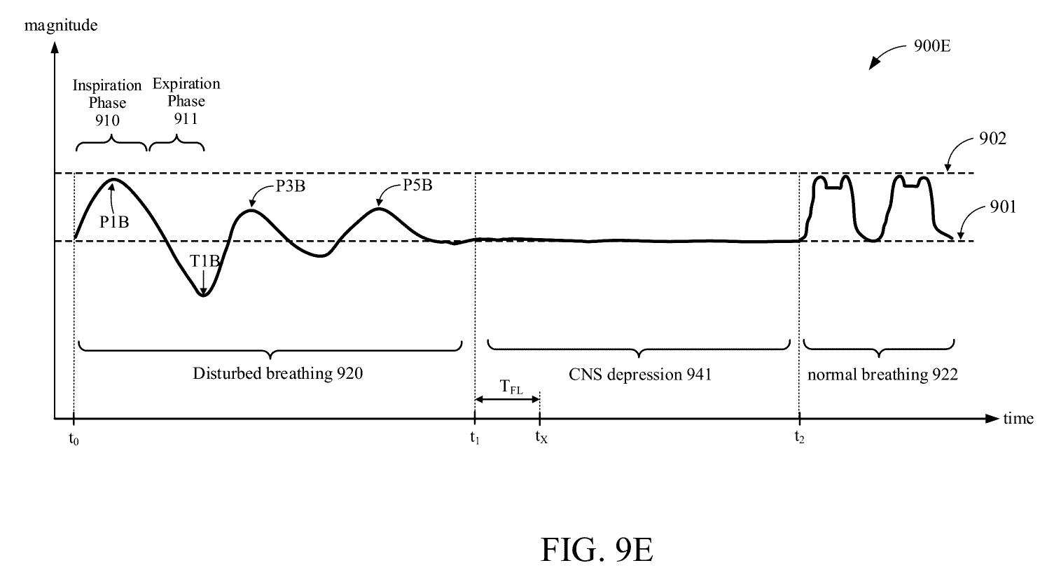

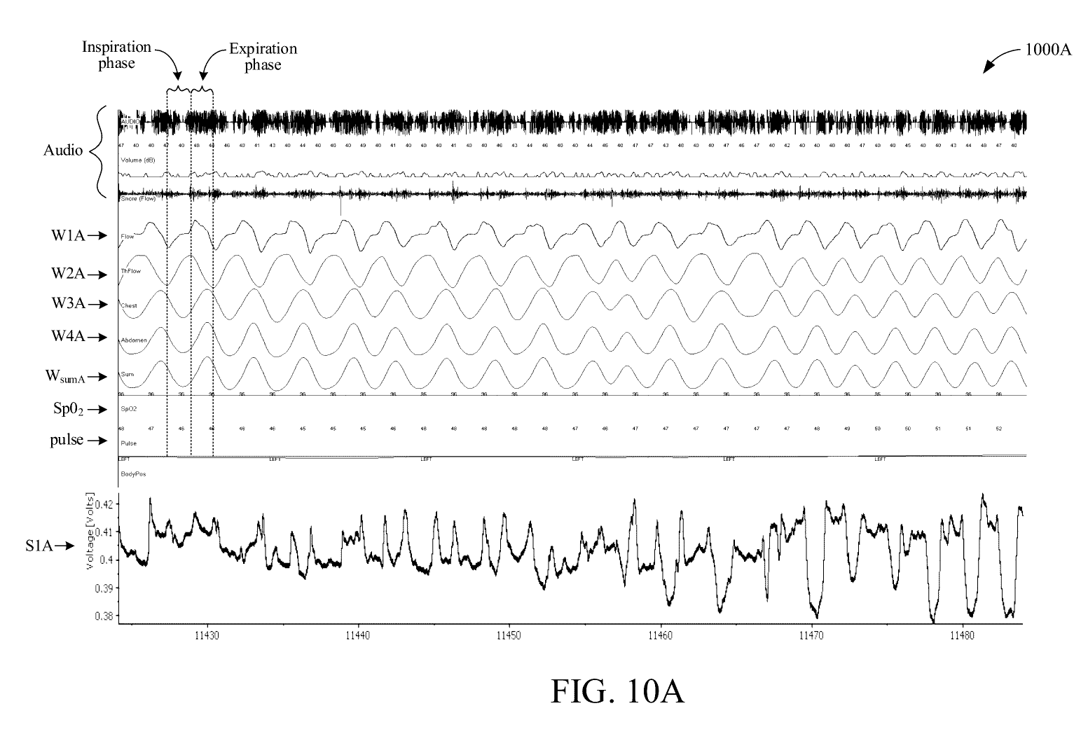

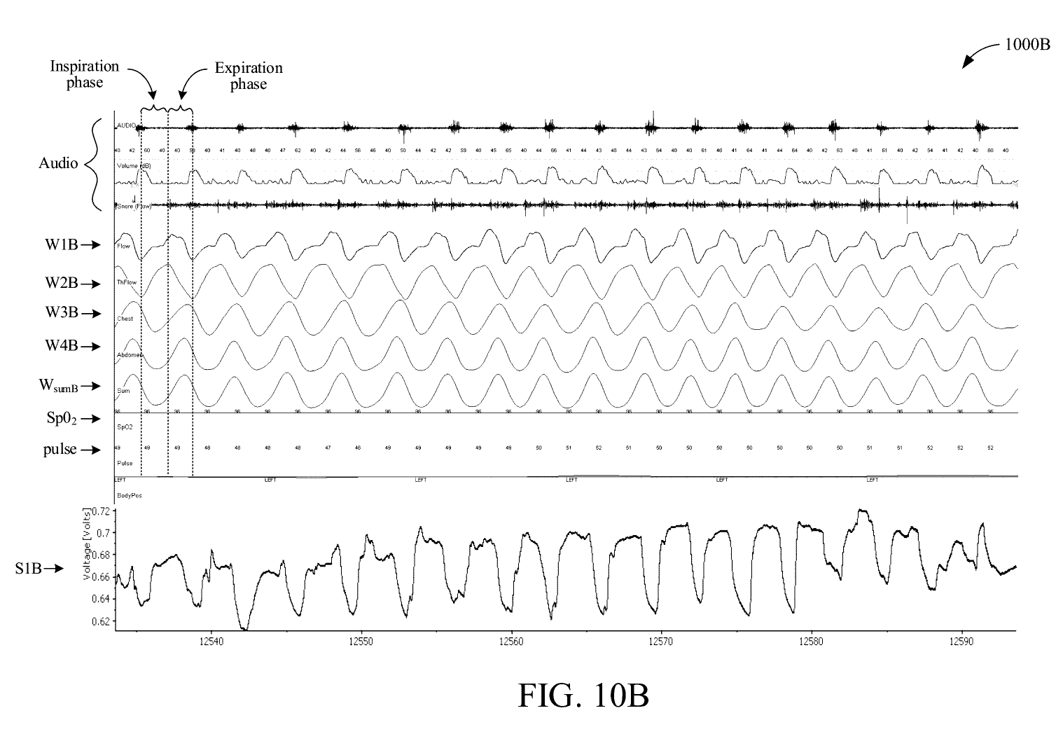

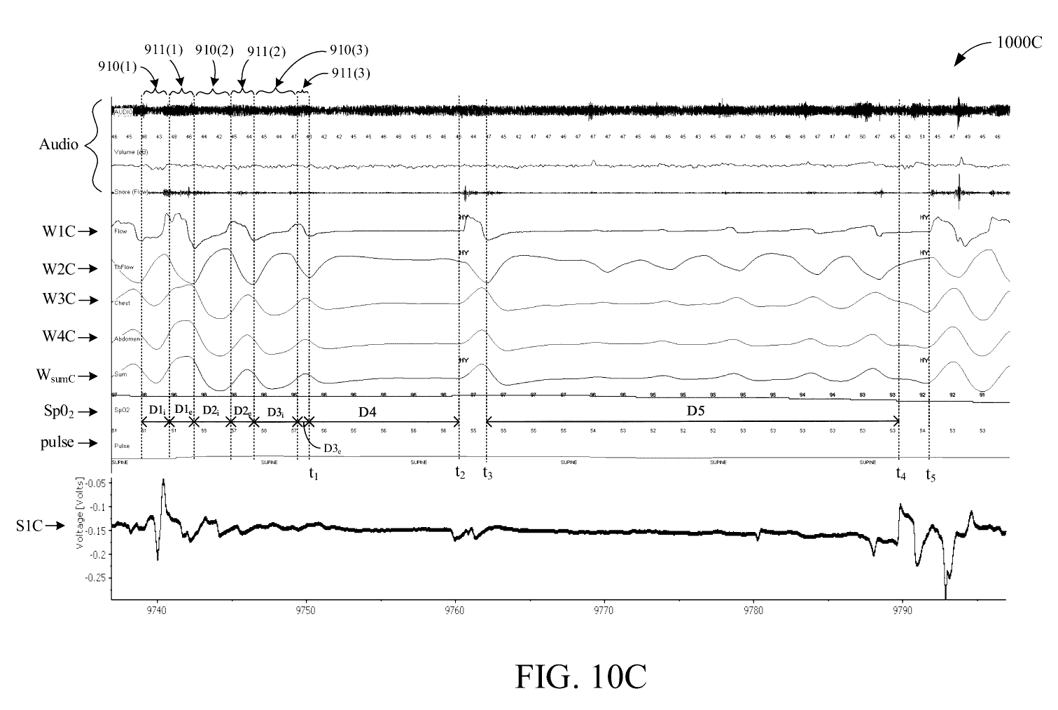

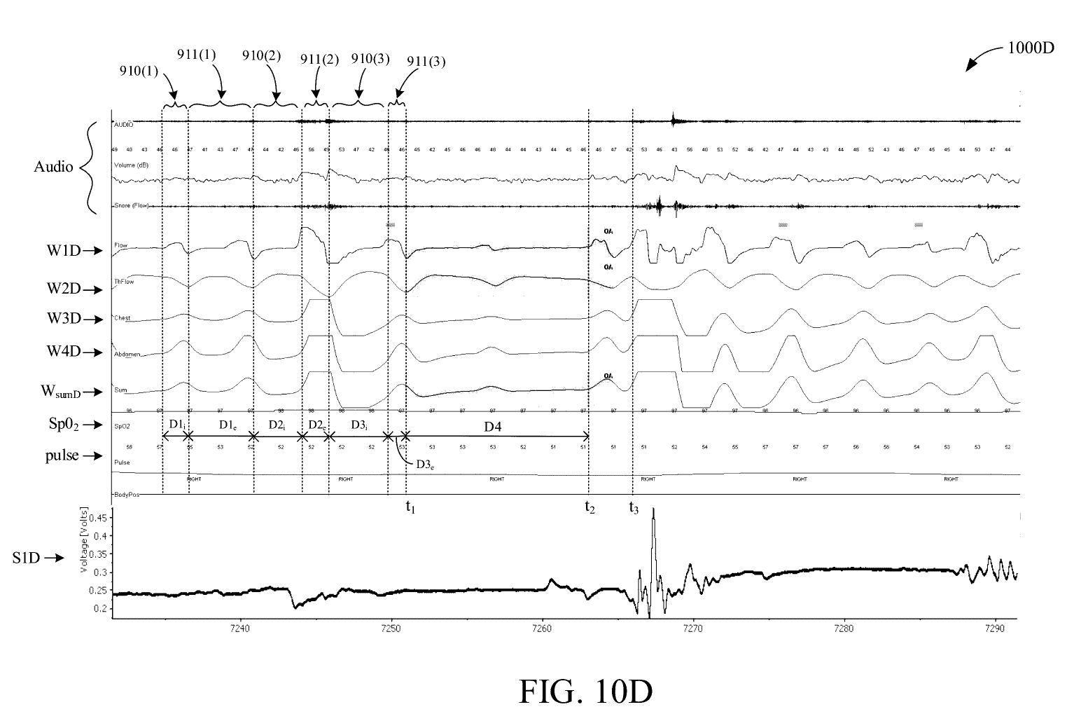

In accordance with the example embodiments, devices and methods are disclosed that may monitor a state of a patient's upper airway and predict an onset and/or detect a presence of disordered breathing in the patient based, at least in part, on the monitored state. For example, the disordered breathing may include a breathing obstruction, a central nervous system (CNS) depression, and/or an abnormal respiration of the patient. The breathing obstruction may include one or more types of apnea such as, for example, obstructive sleep apnea (OSA) and hypopnea, and may be accompanied by snoring. The abnormal respiration may include hyperventilation or hypoventilation of the patient. More specifically, the devices and methods disclosed herein may monitor the respiratory rate, the respiratory effort, and/or the respiratory patterns of a patient. Respiratory rate may indicate how frequently the patient is breathing, and respiratory effort may indicate how much energy the patient is using to breathe. The respiratory patterns may be used to predict the onset and/or to detect the presence of disordered breathing, for example, as described in more detail below with respect to FIGS. 9A-9E and 10A-10D.

The devices and methods disclosed herein may determine a type of the disordered breathing based, at least in part, on the monitored state. For one example, the devices and methods disclosed herein may determine whether the disordered breathing results from a breathing obstruction or from CNS depression. The ability to distinguish between a breathing obstruction and CNS depression may be important in acute care situations to quickly determine a proper treatment or course of action, as described in more detail below. For another example, the devices and methods disclosed herein may detect changes in respiration and determine whether the abnormal respiration results from hyperventilation or from hypoventilation of the patient, which may also be important in acute care situations to quickly determine a proper treatment or course of action, as described in more detail below.

The state of the patient's upper airway may be monitored using a number of contacts provided within (or inserted at least partially within) the patient's oral cavity. In some aspects, the monitored state may include (or be characterized by) one or more electrical signals associated with the patient's upper airway. The one or more electrical signals may be indicative of electrical activity in the patient's upper airway, movement of the patient's upper airway, and a change in the patient's respiration rate. For some implementations, the one or more electrical signals may be used to initiate, adjust, and/or terminate electrical stimulation applied to one or more portions of the patient's oral cavity or upper airway. The electrical stimulation may be used to maintain upper airway patency, for example, as described above. These and other details of the example embodiments, which provide one or more technical solutions to the aforementioned technical problems, are described in more detail below.

For example, FIG. 8A shows a device 800 configured to monitor a state of a patient's upper airway and to sense one or more other indicators of the patient's respiration state or other suitable physiological states in accordance with example embodiments. Device 800 may be another embodiment of the appliance 200 described above with respect to FIGS. 2A-2D. The device 800 of FIG. 8A may be similar to the appliance 200 of FIGS. 2A-2D, except that the device 800 includes a number of contacts 810(1)-810(2) instead of electrodes 210(1)-210(2) and sensors 240(1)-240(2) of appliance 200, and includes a control circuit 820 instead of control circuit 220. Although only two contacts 810(1)-810(2) are shown in the example of FIG. 8A for simplicity, it is to be understood that for other embodiments, device 800 may include any suitable number of contacts such as contacts 810(1)-810(2). The example of FIG. 8A depicts contacts 810(1)-810(2) as being positioned near the patient's last molar teeth (or would be if the patient no longer has the last molars) and extending posteriorly beyond the last molars when device 800 is inserted within a patient's oral cavity, for example, so that contacts 810(1)-810(2) can make electrical contact (and, in some implementations, physical contact) with the patient's lingual tissues proximate to the lateral points at which the Palatoglossus muscle inserts into the patient's tongue (see also FIGS. 1A-1B).

As discussed above, the positioning of at least a portion of contacts 810(1)-810(2) on opposite sides of the patient's tongue posteriorly beyond the last molars (or locations at which the last molars were or should have been) of the patient can be critical to electrically stimulate the patient's Palatoglossus muscle without targeting the patient's Hypoglossal nerve or the patient's genioglossus muscle. Further, for at least some implementations, the contacts 810(1)-810(2) can be angularly oriented with respect to the floor of the patient's mouth to allow the contacts 810(1)-810(2) to substantially face opposite sides of the tongue proximate to the lateral points at which the Palatoglossus muscle inserts into the tongue. However, it is to be understood that for other embodiments, contacts 810(1)-810(2) may be located on, attached to, or otherwise coupled to other suitable portions of device 800.

For other implementations, a pair of devices device 800 may be used together to detect one or more states of the patient and to provide electrical stimulation therapy to the patient. More specifically, a first device 800 may be positioned over the patient's lower teeth (as depicted in FIG. 8A), and a second device 800 may be adapted to fit over the patient's upper teeth. In this manner, one or more of the sensors 840 may be provided on the second device 800 adapted to fit over the patient's upper teeth.

The contacts 810(1)-810(2) may be any suitable contact (e.g., a surface electrode) that may be used to monitor a state of the patient's upper airway, to provide one or more signals indicative of the monitored state of the patient's upper airway, and/or to provide electrical stimulation to one or more portions of the patient's upper airway (e.g., as described above with respect to appliance 200 and/or appliance 700). As mentioned above, the one or more signals provided by the contacts 810(1)-810(2) may be indicative of electrical activity of musculature, nerves, and/or tissues of (or associated with) the patient's upper airway, may be indicative of movement of the patient's upper airway, and/or may be indicative of the respiration of the patient. In some aspects, the one or more signals provided by the contacts 810(1)-810(2) may be EMG signals. In other aspects, the one or more signals provided by the contacts 810(1)-810(2) may be electroencephalogram (EEG) signals.

The control circuit 820, which may include a number of components and/or features of control circuit 220 described above with respect to FIGS. 2A-2D, may be coupled to contacts 810(1)-810(2) via conductive wires 221 (other any other suitable electrical connection). The control circuit 820 may predict and/or detect an onset of disordered breathing in a patient based, at least in part, on the monitored state of the patient's upper airway. More specifically, in some implementations, the control circuit 820 may analyze the one or more signals provided by contacts 810(1)-801(2) to predict and/or detect an onset of disordered breathing such as, for example, a breathing obstruction, respiratory distress such as central nervous system (CNS) depression, snoring, and/or an abnormal respiration of the patient. The breathing obstruction may include one or more types of apnea such as, for example, obstructive sleep apnea (OSA) and hypopnea, and may be accompanied by snoring. The abnormal respiration may include hyperventilation or hypoventilation of the patient.

The device 800 may include or be coupled to a number of sensors 840. It is to be understood that the position of sensors 840 in the example of FIG. 8A is merely illustrative; for actual embodiments, sensors 840 may be positioned in any suitable manner or at any feasible location within or outside the patient's oral cavity. The sensors 840 may detect any number of respiration attributes of the patient including, for example, vibration of the patient's upper airway, sounds of the patient's upper airway, airflow in the patient's upper airway, an oxygenation rate of the patient, a level of carbon dioxide of the patient, a heartrate of the patient, or any other suitable indications of the patient's respiration. The sensors 840 may be configured to provide signals indicative of one or more of these respiration attributes to the control circuit 820. In some aspects, one or more of the sensors 840 may be or include a thermistor that measures airflow through the patient's oral cavity. In other aspects, one or more of the sensors 840 may be or include a constant current oscillator that measures or determines charging and discharging times of the patient's tongue, which as described in more detail below may be used to predict the onset of various disordered breathing conditions, to determine the presence of various disordered breathing conditions, and/or to determine various levels of sleep or consciousness of the patient.

The control circuit 820 may also be coupled to one or more external sensors via any suitable wired or wireless connection. For purposes of discussion herein, these external sensors are positioned external to the patient's oral cavity, and are not shown in FIG. 8A for simplicity. The external sensors, which are depicted in and descried below with respect to FIG. 8B, may provide signals indicating, for example, movement of the patient's chest (e.g., indicating volume changes in response to inspiration and expiration of the patient), movement of the patient's abdomen, movement of the patient's jaw, or any combination thereof. Thus, for at least some implementations, the control circuit 820 may determine the type of disordered breathing based, at least in part, on the one or more signals provided by the contacts 810(1)-810(2), the signals provided by the sensors 840, the signals provided by the external sensors, or any combination thereof. More specifically, the control circuit 820 may determine whether the disordered breathing results from a breathing obstruction, respiratory distress, or hyperventilation based, at least in part, on the one or more signals provided by contacts 810(1)-810(2), the signals generated by the sensors 840, the signals generated by the external sensors, or any combination thereof. The control circuit 820 may be configured to generate an alert indicating whether the disordered breathing results from a breathing obstruction or from CNS depression. The alert may be used by medical personnel in acute care situations to quickly determine a proper treatment of course of action.

The ability to quickly distinguish between a breathing obstruction and respiratory distress may be particularly important in hospitals or other urgent care environments in which the cause, and thus the appropriate treatment, of a patient's disordered breathing must be quickly determined to safeguard the patient's well-being. It is noted that patients suffering from OSA and/or snoring may have an increased risk of breathing difficulties when awaking from sleep induced by general anesthetics.

For example, patients are typically given a general anesthetic prior to many surgical procedures. After surgery, the patient is typically placed in a post-op room until the patient wakes up from the sleep state induced by general anesthetics. Although the patient is typically monitored for disordered breathing after surgery, it may be difficult to determine whether the disordered breathing is caused by a breathing obstruction (e.g., a prolapsed tongue or a collapsed palate) or by respiratory distress (e.g., CNS depression), thereby rendering it difficult to quickly determine the proper course of treatment.

More specifically, if the patient's post-op disordered breathing is caused by a breathing obstruction, then proper treatment may involve removing the obstruction (e.g., by moving the patient's tongue forward). Conversely, if the patient's post-op disordered breathing is caused by CNS depression, then proper treatment may involve ventilation of the patient (e.g., using a bag valve mask or a respirator). Selecting the wrong course of treatment for the patient's disordered breathing may result in serious harm to (or even death of) the patient. For example, if the patient has a breathing obstruction and the medical personnel incorrectly opt to place a bag valve mask over the patient's mouth (e.g., in an attempt to ventilate the patient), the patient may suffocate. Conversely, if the patient has CNS depression and the medical personnel incorrectly opt to manually move the patient's tongue forward (e.g., in an attempt to remove the breathing obstacle, rather than treating the CNS depression), the patient may lose consciousness, possibly leading to coma or death. Thus, the ability of the example embodiments to quickly and accurately distinguish between a breathing obstruction and CNS depression may prevent unnecessary harm and/or loss of life of patients recovering from surgical procedures.

In another implementation, the control circuit 820 may analyze the one or more signals provided by contacts 810(1)-810(2), the signals generated by the sensors 840, the signals generated by the external sensors, or any combination thereof to detect a level of consciousness of the patient or to detect a change in the level of consciousness of the patient. In some aspects, the determined level of consciousness may be indicative of a depth of anesthesia, for example, given to a patient undergoing a medical procedure. The control circuit 820 may generate an alert indicating the level of consciousness of the patient. Medical personnel may use the indicated level of consciousness to determine whether to apply additional anesthesia to the patient (e.g., so that the patient does not prematurely wake during the medical procedure).

In other aspects, the determined level of consciousness may be indicative of various sleep states of a patient (e.g., waking up, falling asleep, losing consciousness) and to determine various levels or stages of sleep (e.g., REM sleep). For example, in some embodiments, a determined level of sleep may be used to allow and/or prevent the application of electrical stimulation to one or more portions of the patient's upper airway. More specifically, if the control circuit 820 determines that the patient is not asleep, then the control circuit 820 may prevent electrical stimulation of the patient's upper airway; conversely, if the control circuit 820 determines that the patient is asleep, then the control circuit 820 may allow electrical stimulation of the patient's upper airway based on the monitored state of the patient's upper airway (e.g., as described above with respect to FIGS. 2A-2D, 3A-3B, and 4-6).

In yet another implementation, the control circuit 820 may analyze the one or more signals provided by contacts 810(1)-810(2), the signals generated by the sensors 840, the signals generated by the external sensors, or any combination thereof to monitor the respiration of the patient and/or to detect a change in respiration of the patient. The monitored respiration may include a respiration rate, an inspiratory effort, and/or respiration patterns of a patient. The respiration rate may indicate how frequently the patient is breathing, and the inspiratory effort may indicate how much energy the patient is using to breathe. The respiration patterns may be used to predict the onset and/or to detect the presence of disordered breathing.

The control circuit 820 may initiate, adjust, and/or terminate the electrical stimulation of one or more portions of the patient's upper airway based on the one or more signals provided by contacts 810(1)-810(2), the signals generated by the sensors 840, the signals generated by the external sensors, or any combination thereof. More specifically, the control circuit 820 may vary the type, frequency, magnitude, polarity, and/or pattern of electrical stimulation provided by the contacts 810(1)-810(2) based on the one or more signals provided by the contacts 810(1)-810(2), the signals generated by one or more sensors 840, the signals generated by one or more external sensors, or any combination thereof. Thus, in addition to predicting or detecting the onset of disordered breathing and/or distinguishing between a breathing obstruction and respiratory distress, the device 800 may provide immediate corrective action to at least some types of disordered breathing. For example, if the one or more signals provided by contacts 810(1)-810(2), the signals generated by one or more sensors 840, the signals generated by one or more external sensors, or any combination thereof indicate a breathing obstruction, the device 800 may be configured to automatically increase the patient's upper airway patency (e.g., via electrical stimulation, as described above) or to automatically awake the patient (e.g., by increasing the magnitude and/or duration of electrical stimulation to a level that forces the patient to wake up).

The device 800 may be inserted (at least partially) into a patient's oral cavity such that the contacts 810(1)-810(2) are positioned in a manner to provide one or more signals that can be used to monitor a state of the patient's upper airway. The monitored state may include a number of attributes including (but not limited to) muscle tone, muscle movement, relative tension of the tongue (e.g., relaxed tongue muscles versus tensed tongue muscles), nerve activity, motor unit function, tonic/phasic activity of the tongue, and/or electrical activity of the musculature, nerves, and/or tissues associated with the patient's upper airway.

To effectively predict or detect an onset of disordered breathing, distinguish between breathing obstructions and CNS depression, detect an onset of snoring, detect changes in respiration, determine levels of consciousness, and/or determine a depth of sleep, the electrical activity of a patient's upper airway musculature, nerves, and/or tissue must be detected with a level of accuracy typically associated with intramuscular EMG techniques; the level of accuracy provided by conventional surface EMG techniques may be insufficient to achieve the benefits of the example embodiments described herein. Indeed, prior to this disclosure, the use of surface EMG was not expected to be able to detect the electrical activity of a patient's upper airway musculature, nerves, and/or tissue with the level of accuracy and resolution necessary for the example embodiments to provide solutions to the aforementioned technical problems. However, Applicant has discovered that when contacts 810(1)-810(2) are properly positioned within a patient's oral cavity, a number of electrically conductive properties of the oral cavity may amplify electrical activity associated with a patient's upper airway in a manner that allows contacts 810(1)-810(2) to detect or generate the one or more signals with a level of accuracy typically associated with intramuscular EMG. Indeed, the contacts 810(1)-810(2) of device 800 may detect electrical activity of the patient's upper airway musculature, nerves, and/or tissue with none of the signal degradation that would be expected by those skilled in the art. Indeed, in some aspects, the signals detected or generated by contacts 810(1)-810(2) of device 800 may be cleaner (e.g., more robust and less susceptible to interference) than conventional EMG signals.

More specifically, after testing the electrical properties of various portions of a patient's oral cavity, Applicant has found that the tongue has an intrinsic capacitance on the order of approximately 0.3 microfarads. This intrinsic capacitance allows the tongue to store or retain an electrical charge, which in turn may amplify electrical activity associated with the patient's upper airway to a level sufficient for contacts 810(1)-810(2) to detect or generate one or more electrical signals that may be used to accurately monitor one or more states of the patient's upper airway. In addition, Applicant has found that human saliva exhibits electrically conductive properties and may couple electrical signals generated by the patient's upper airway musculature and nerves to contacts 810(1)-810(2), thereby further increasing the ability of device 800 to monitor one or more states of the patient's upper airway based on electrical signals detected or generated by contacts 810(1)-810(2).

The intrinsic capacitance of the tongue may also allow the example embodiments to utilize capacitive sensing techniques to gather information regarding a patient's physiological condition, Palatoglossus muscle enervation and movement, and tongue enervation and movement. More specifically, in accordance with example embodiments, a constant current oscillator may be coupled to the patient's tongue or sublingual tissues, and configured to provide a current that repeatedly charges and discharges the intrinsic capacitive element of the tongue (the intrinsic capacitive element of the tongue may hereinafter be referred to as the “tongue capacitor”). The capacitance of the tongue changes based on various physical and biological characteristics of the tongue (e.g., the tongue's muscle tone). As the capacitance of the tongue varies, the time required to charge the tongue capacitor also changes, thereby changing an associated oscillation frequency. The varying capacitance of the tongue, which may be measured by analyzing the time it takes to charge and discharge the tongue capacitor, may be used by the example embodiments to predict the onset of breathing abnormalities, to detect and/or distinguish between a breathing obstruction and respiratory distress, and/or other indicators of a patient's respiration activity and/or level of sleep, for example, as described in more detail below.

For example, charging and discharging time may be determined by integrating a square waveform indicative of the tongue's capacitance, where the width of the square waveform (dt) indicates the charging/discharging time. The charging/discharging durations (dts) may be converted to an analog voltage signal using a Digital-to-Analog Converter (DAC). The resultant analog voltage may be plotted versus time to create an analog waveform representing the capacitance oscillator. The amplitude of the resulting “capacitance oscillator waveform” changes with tongue activity, and may therefore be used to detect changes in the movement, tone, and other states of the tongue.

FIG. 8B shows a block diagram of an example embodiment of the control circuit 820. The control circuit 820 is shown to include a monitoring system 850, a stimulation waveform generator 860, a processor 870, a memory 880, and a transceiver 890. Further, although not shown in FIG. 8B for simplicity, control circuit 820 may also include a contact interface circuit and a power supply. The contact interface circuit may be used to route signals from a number of contacts 810, a number of sensors 840, and a number of external sensors 845 to monitoring system 850, and to route signals from stimulation waveform generator 860 to contacts 810. The power supply, which may be one embodiment of power supply 230 of FIG. 4, may provide power to control circuit 820.

The external sensors 845, which may be any suitable one or more sensors capable of detecting or measuring respiration or physiological states of the patient, can be electrically coupled to the control circuit 820 using any suitable wired or wireless connection. For some implementations, the external sensors 845 may include accelerometers, radar devices, or any other suitable devices that can detect movement of the patient's chest, movement of the patient's abdomen, and/or movements in the patient's jaw. In some aspects, the external sensors 845 can include an EMG sensor that monitors a state (e.g., movement, muscle enervation, stimulated nerves, and so on) of the patient's abdomen and provides signals indicative of the monitored state to the control circuit 820.

As used herein, the signals provided by contacts 810 (e.g., indicative of the monitored state of the patient's upper airway), the signals provided by sensors 840 (e.g., indicative of airflow through the patient's upper airway, indicative of charging and discharging times of the patient's tongue, and so on), and the signals provided by external sensors 845 (e.g., indicative of movement of the patient's chest, movement of the patient's abdomen, movements in the patient's jaw, and so on) may collectively be referred to as the “sensing information.” Thus, for at least some implementations, the device 800 may use the sensing information to determine one or more states (e.g., physical, physiologic, and/or respiratory) of the patient, and then configure the electrical stimulation to be applied to the patient based on the one or more determined states.

The monitoring system 850 is coupled to the number of contacts 810, to the number of sensors 840, to the number of external sensors 845, to processor 870, and to memory 880. For the example embodiment of FIG. 8B, the monitoring system 850 is shown to include a disordered breathing prediction circuit 851, a snoring detection circuit 852, a respiration monitoring circuit 853, an apnea detection circuit 854, a level of consciousness determination circuit 855, and a disordered breathing type circuit 856. The disordered breathing prediction circuit 851 may be used to monitor a state of the patient's upper airway and predict an onset of a disordered breathing in the patient based, at least in part, on signals provided by the contacts 810, signals provided by the sensors 840, and/or signals provided by the external sensors 845. In response to an indication of the onset of disordered breathing, the control circuit 820 may cause a number of the contacts 810 to provide an electrical stimulation pattern configured to prevent the onset of disordered breathing. For one example, if the control circuit 820 detects an onset of apnea in the patient, then the control circuit 820 may cause contacts 810 to electrically stimulate one or more portions of the patient's oral cavity in a manner that prevents the onset of apnea (or at least reduces the severity of the apnea). For another example, if the control circuit 820 detects an onset of hyperventilation in the patient, then the control circuit 820 may cause contacts 810 to electrically stimulate one or more portions of the patient's oral cavity in a manner that prevents the onset of hyperventilation (or at least reduces the severity of the hyperventilation).

The snoring detection circuit 852 may be used to monitor a state of the patient's upper airway and detect an onset of snoring in the patient based, at least in part, on signals provided by the contacts 810, signals provided by the sensors 840, and/or signals provided by the external sensors 845. In response to an indication of the onset of snoring in the patient, the control circuit 820 may cause a number of the contacts 810 to provide an electrical stimulation pattern configured to prevent the onset of snoring.

The respiration monitoring circuit 853 may be used to monitor a state of the patient's upper airway and detect a change in respiration of the patient based, at least in part, on signals provided by the contacts 810, signals provided by the sensors 840, and/or signals provided by the external sensors 845.

The apnea detection circuit 854 may be used to monitor a state of the patient's upper airway and detect an occurrence of an apnea in the patient based, at least in part, on signals provided by the contacts 810, signals provided by the sensors 840, and/or signals provided by the external sensors 845. In response to an indication of the onset of apnea, the control circuit 820 may cause a number of the contacts 810 to provide an electrical stimulation pattern configured to prevent the onset of apnea (or at least reduces the severity of the apnea).

The level of consciousness determination circuit 855 may be used to monitor a state of the patient's upper airway and determine a level of consciousness of the patient based, at least in part, on signals provided by the contacts 810, signals provided by the sensors 840, and/or signals provided by the external sensors 845.

The disordered breathing type circuit 856 may be used to determine a type of disordered breathing in the patient based, at least in part, on signals provided by the contacts 810, signals provided by the sensors 840, and/or signals provided by the external sensors 845.

The stimulation waveform generator 860 is coupled to contacts 810 and to processor 870. The stimulation waveform generator 860 may generate and/or dynamically adjust electrical signals provided to the contacts 810, for example, to cause the contacts 810 to provide various types or patterns of electrical stimulation to one or more portions of the patient's upper airway. The electrical stimulation may be used to maintain upper airway patency, for example, as described above with respect to FIGS. 2A-2D, 3A-3B, and 4-6. For some implementations, the electrical stimulation provided by the contacts 810 may be initiated, adjusted, and/or terminated by stimulation waveform generator 860 based, at least in part, on one or more feedback (FB) signals provided by the monitoring system monitoring system 850. For other implementations, the electrical stimulation provided by the contacts 810 may be continuous, for example, as part of an open loop system.

Memory 880 may include a data store 889. The data store 889 may store information including profile information for a number of patients. The profile information for a given patient may include, for example, information pertaining to previous occasions for which device 800 was used to monitor one or more states of the patient's upper airway (e.g., the prediction or detection of disordered breathing, apnea, respiration, level of consciousness, and/or depth of sleep), information pertaining to previous applications of electrical stimulation based on the one or more monitored states, information pertaining to the effectiveness or results of the previous applications of electrical stimulation, the patient's level of compliance in using device 800, medical history of the patient, one or more reference signals or waveforms of the patient, and/or any other information that may be relevant to the patient.

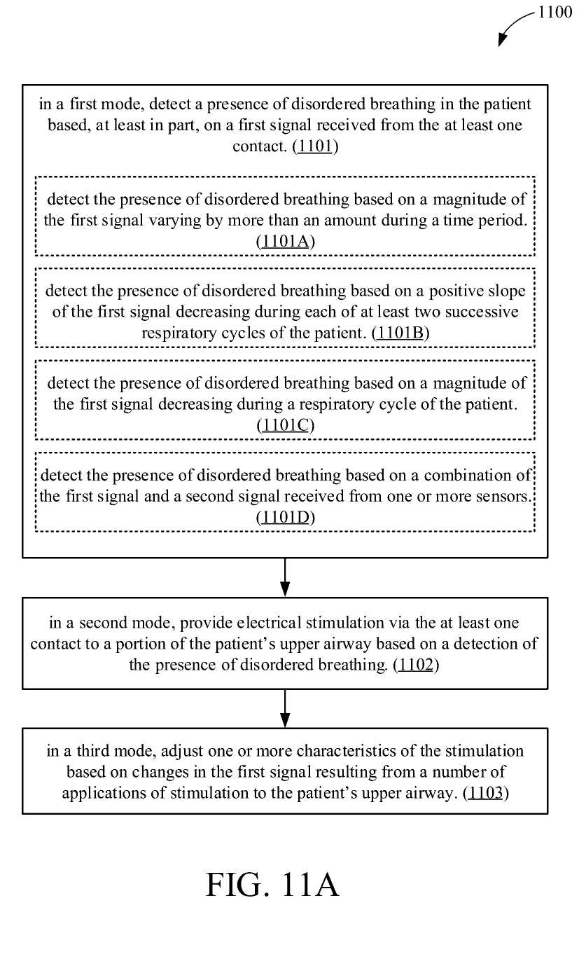

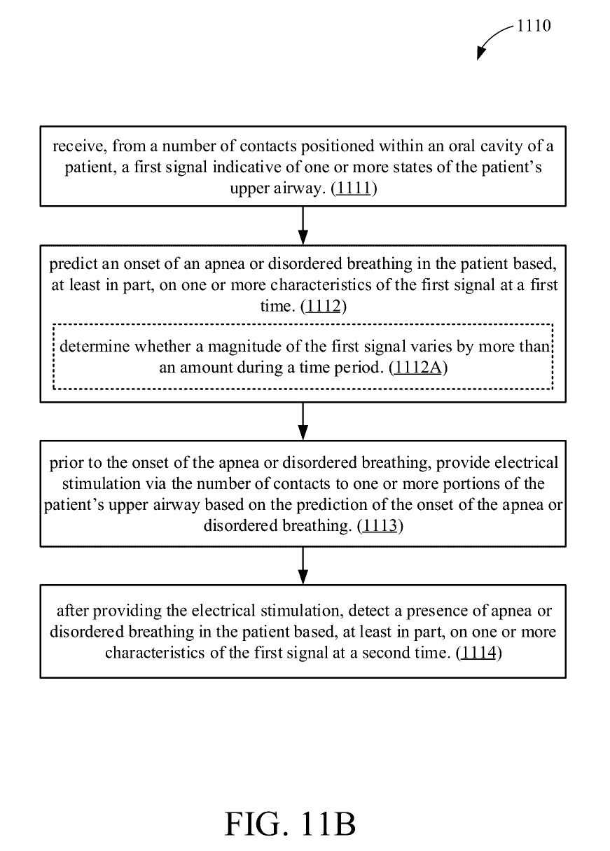

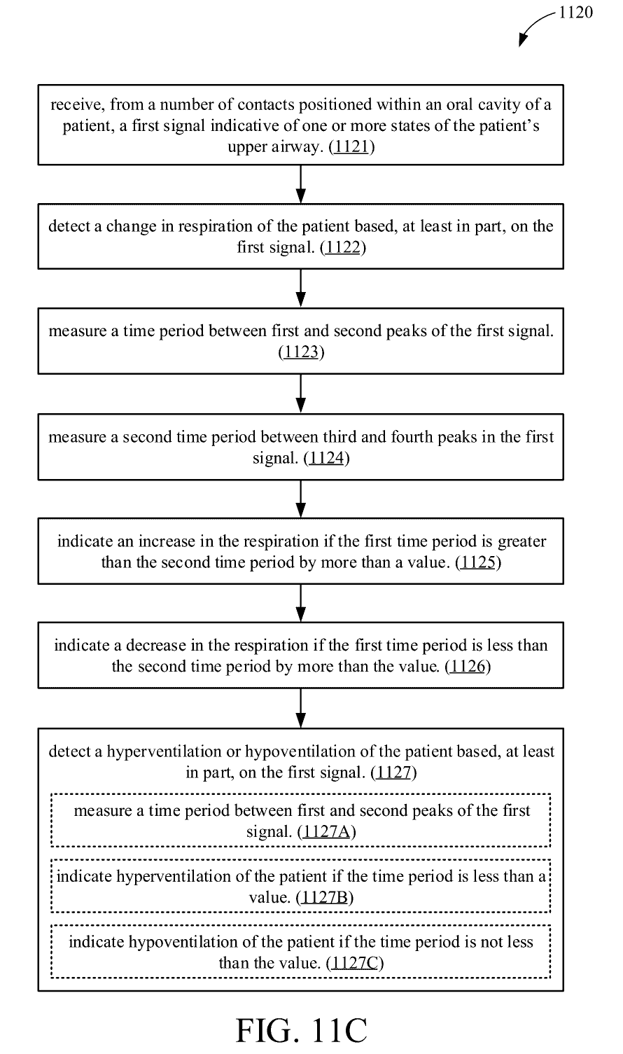

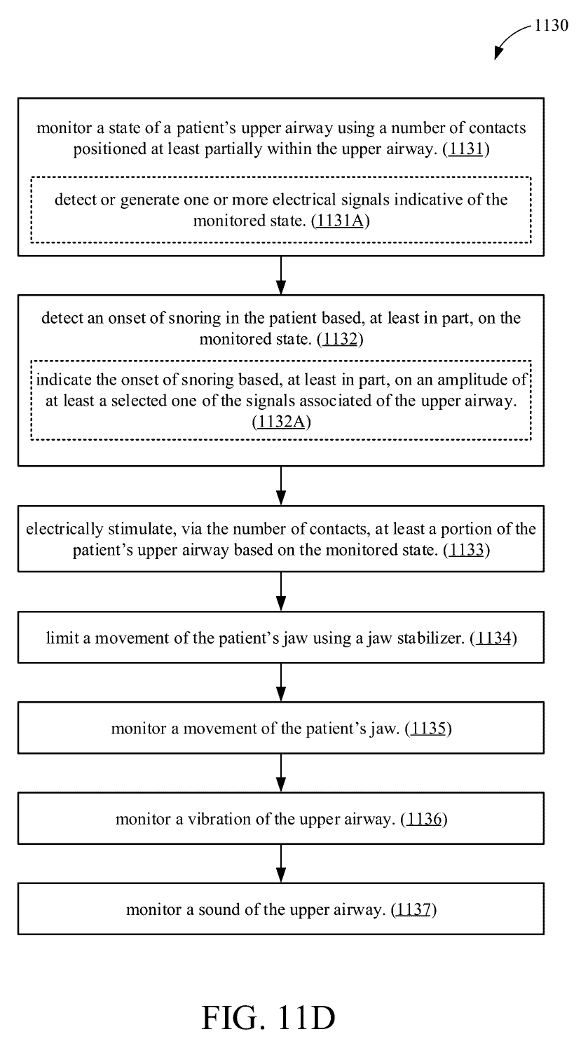

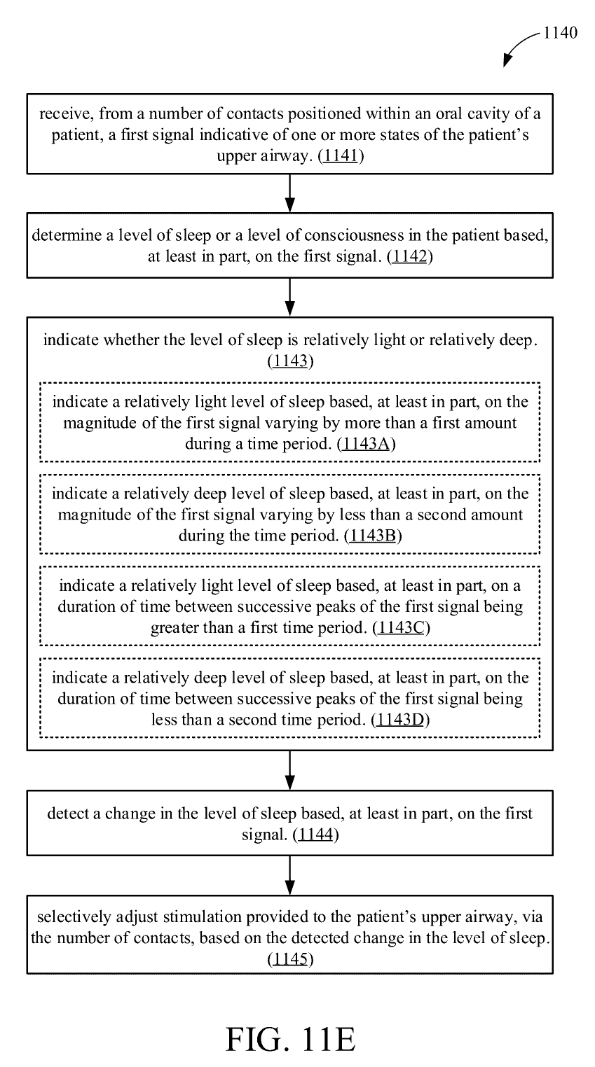

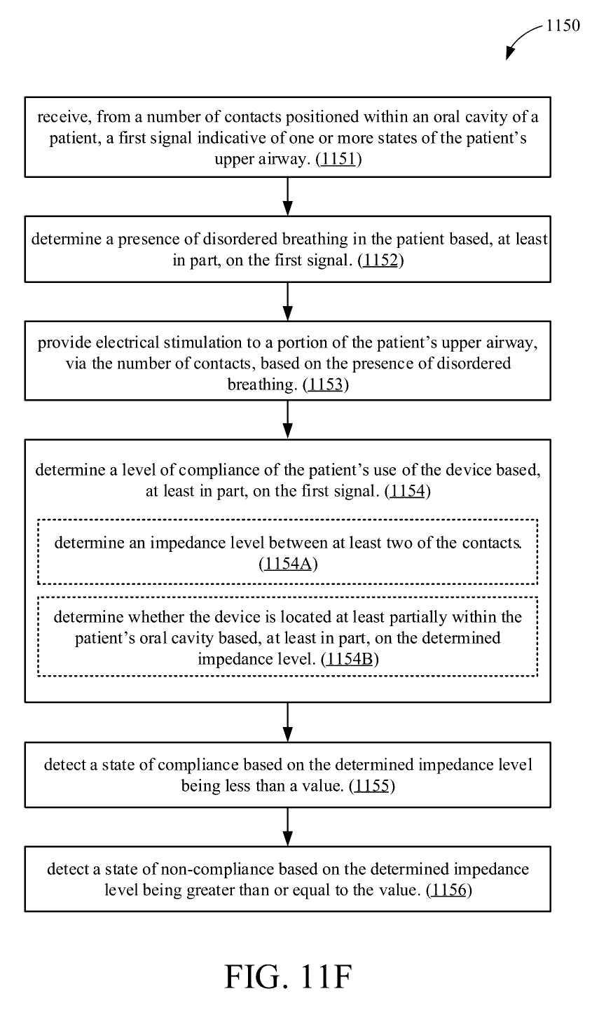

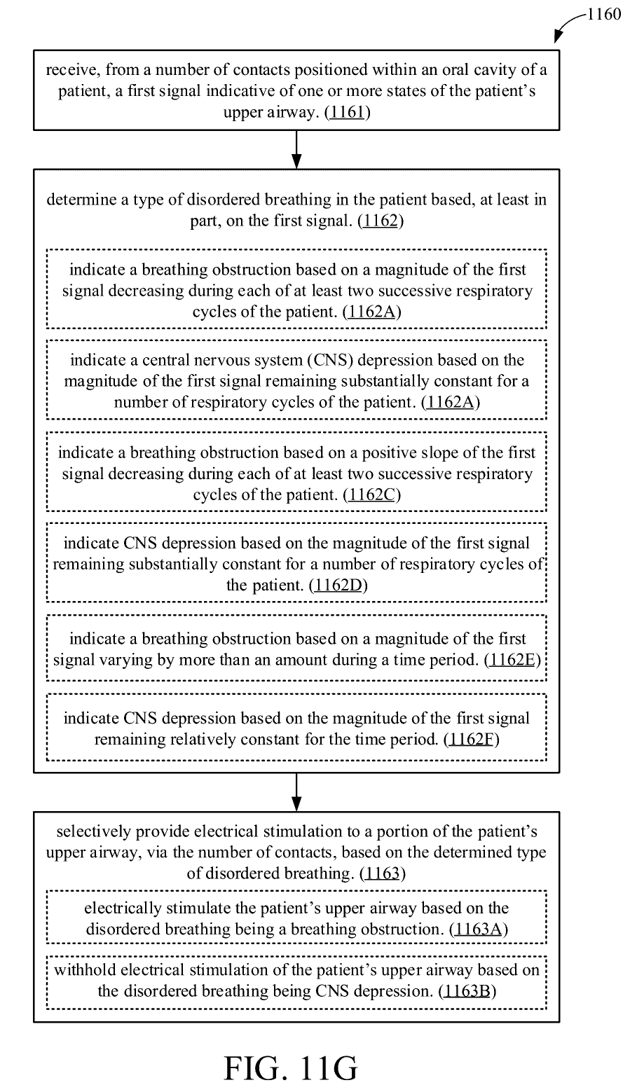

Memory 880 may also include a non-transitory computer-readable storage medium (e.g., one or more nonvolatile memory elements, such as EPROM, EEPROM, Flash memory, a hard drive, etc.) that may store at least the following software (SW) modules:
a disordered breathing detection SW module 881 to detect and treat disordered breathing in a patient's upper airway based, at least in part, on signals provided by the contacts 810, signals provided by the sensors 840, and/or signals provided by the external sensors 845 (e.g., as described below for one or more operations of FIG. 11A);
a snoring detection SW module 882 to monitor a state of the patient's upper airway and detect an onset of snoring in the patient based, at least in part, on signals provided by the contacts 810, signals provided by the sensors 840, and/or signals provided by the external sensors 845 (e.g., as described below for one or more operations of FIG. 11D);
a respiration monitoring SW module 883 to detect a change in respiration of the patient based, at least in part, on signals provided by the contacts 810, signals provided by the sensors 840, and/or signals provided by the external sensors 845 (e.g., as described below for one or more operations of FIG. 11C);
an apnea prediction SW module 884 to predict an onset of an apnea or disordered breathing in the patient based, at least in part, on signals provided by the contacts 810, signals provided by the sensors 840, and/or signals provided by the external sensors 845 (e.g., as described below for one or more operations of FIG. 11B);
a level of sleep/consciousness determination SW module 885 to determine a level of sleep or a level of consciousness of the patient based, at least in part, on signals provided by the contacts 810, signals provided by the sensors 840, and/or signals provided by the external sensors 845 (e.g., as described below for one or more operations of FIG. 11E);
a disordered breathing type determination SW module 886 to determine a type of disordered breathing in the patient based, at least in part, on signals provided by the contacts 810, signals provided by the sensors 840, and/or signals provided by the external sensors 845 (e.g., as described below for one or more operations of FIG. 11G);
a capacitive oscillator SW module 887 to receive, from the contacts 810, signals indicating charging and discharging times of the patient's tongue and to predict the onset of various disordered breathing conditions, to determine the presence of various disordered breathing conditions, and/or to determine various levels of sleep or consciousness of the patient based on changes in the charging and discharging times of the patient's tongue; and
a compliance SW module 888 to determine a level of compliance of the patient's use of device 800 based, at least in part, on signals received from the contacts the contacts 810 (e.g., as described below for one or more operations of FIG. 11F). For some implementations, execution of the compliance SW module 888 may determine an impedance level between at least two of the contacts 810, and then determine whether the device is located at least partially within the patient's oral cavity based, at least in part, on the determined impedance level.

Each software module may include instructions that, when executed by the processor 870, may cause device 800 of FIG. 8A to perform the corresponding function. Thus, the non-transitory computer-readable storage medium of memory 880 may include instructions for performing all or a portion of the operations described below with respect to FIGS. 11A-11G. The processor 870 may be any suitable one or more processors capable of executing scripts of instructions of one or more software programs stored in the device 800 (e.g., within memory 880). For example, the processor 870 may execute the disordered breathing detection SW module 881 to detect and treat disordered breathing in a patient's upper airway based, at least in part, on signals provided by the contacts 810, signals provided by the sensors 840, and/or signals provided by the external sensors 845. In some aspects, execution of the disordered breathing prediction SW module 881 may perform operations similar to those described above with respect to the disordered breathing prediction circuit 851.

The processor 870 may execute the snoring detection SW module 882 to monitor a state of the patient's upper airway and detect an onset of snoring in the patient based, at least in part, on signals provided by the contacts 810, signals provided by the sensors 840, and/or signals provided by the external sensors 845. In some aspects, execution of the snoring detection SW module 882 may perform operations similar to those described above with respect to the snoring detection circuit 852.

The processor 870 may execute the respiration monitoring SW module 883 to detect a change in respiration of the patient based, at least in part, on signals provided by the contacts 810, signals provided by the sensors 840, and/or signals provided by the external sensors 845. In some aspects, execution of the respiration monitoring SW module 883 may perform operations similar to those described above with respect to the respiration monitoring circuit 853.

The processor 870 may execute the apnea prediction SW module 884 to predict an onset of an apnea or disordered breathing in the patient based, at least in part, on signals provided by the contacts 810, signals provided by the sensors 840, and/or signals provided by the external sensors 845. In some aspects, execution of the apnea detection SW module 884 may perform operations similar to those described above with respect to the apnea detection circuit 854.

The processor 870 may execute the level of sleep/consciousness determination SW module 885 to determine a level of sleep or a level of consciousness of the patient based, at least in part, on signals provided by the contacts 810, signals provided by the sensors 840, and/or signals provided by the external sensors 845. In some aspects, execution of the level of consciousness determination SW module 885 may perform operations similar to those described above with respect to the level of consciousness determination circuit 855.

The processor 870 may execute the disordered breathing type determination SW module 886 to determine a type of disordered breathing in the patient based, at least in part, on signals provided by the contacts 810, signals provided by the sensors 840, and/or signals provided by the external sensors 845. In some aspects, execution of disordered breathing type determination SW module 886 may perform operations similar to those described above with respect to the disordered breathing type circuit 856.

The processor 870 may execute the capacitive oscillator SW module 887 to receive, from the contacts 810, signals indicating charging and discharging times of the patient's tongue and to predict the onset of various disordered breathing conditions, to determine the presence of various disordered breathing conditions, and/or to determine various levels of sleep or consciousness of the patient based on changes in the charging and discharging times of the patient's tongue.

The processor 870 may execute the compliance SW module 888 to determine a level of compliance of the patient's use of device 800 based, at least in part, on signals received from the contacts 810. More specifically, execution of the compliance SW module 888 may determine an impedance level between at least two of the contacts 810, and then determine whether the device is located at least partially within the patient's oral cavity based, at least in part, on the determined impedance level. For some implementations, execution of the compliance SW module 888 may detect a state of compliance based on the determined impedance level being less than a value, and may detect a state of non-compliance based on the determined impedance level being greater than or equal to the value. In some aspects, execution of the compliance SW module 888 may determine a first portion of a time period associated with the detected state of compliance, determine a second portion of the time period associated with the detected state of non-compliance, and may generate an indication of compliance or non-compliance based on a comparison between the first and second portions.

As depicted in the example of FIG. 8B, the processor 870 may include a mode control circuit 875. Although the mode control circuit 875 is depicted in the example of FIG. 8B as part of the processor 870, for other implementations, the mode control circuit 875 can be separate from and coupled to the processor 870. The mode control circuit 875 can generate a mode signal to indicate a number of different operating modes of the device 800. For some implementations, the operational modes of the device 800 may include a sensing mode, a therapy mode, a calibration mode, and a compliance mode.

The sensing mode may be used to determine one or more respiration states of the patient. These one or more respiration states may be used to predict the onset or occurrence of disordered breathing in the patient, to determine a type of the disordered breathing in the patient, to detect changes in respiration of the patient, to determine levels of consciousness of the patient, to determine a depth of sleep of the patient, and/or to detect or determine other suitable physiologic conditions. More specifically, when the device 800 operates in the sensing mode, the control circuit 820 may receive signals (S1) from contacts 810 that indicate a state of the patient's upper airway, may receive signals (S2) from the sensors 840 that indicate an amount of airflow in the patient's upper airway and/or that indicate a change in the capacitance of the patient's tongue, and may receive signals (S3) from the external sensors 845 that indicate movement of the patient's chest, movement of the patient's abdomen, and/or movements in the patient's jaw. For some implementations, sensing information indicative of airflow in the patient's upper airway, movement of the patient's chest, movement of the patient's abdomen, movements in the patient's jaw, and/or Sp0₂ levels may be provided by a separate device (not shown for simplicity) rather than by the sensors 840 and the external sensors 845. For one example, the separate device may be a Medibyte portable sleep data recorder that records a number of physiological signals of the patient including, for example, airflow in the patient's upper airway, movement of the patient's chest, movement of the patient's abdomen, movements in the patient's jaw, heartrate, and/or SP0₂ levels.

The therapy mode may be used to deliver electrical stimulation therapy to the patient, for example, based on the one or more determined respiration states of the patient. As discussed above, the patient's respiratory state may be determined based on the signals S1 received from contacts 810, on the signals S2 provided by sensors 840, and/or on the signals S3 provided by external sensors 845. More specifically, when the device 800 operates in the therapy mode, the control circuit 820 may cause contacts 810 to provide electrical stimulation to one or more portions of the patient's oral cavity based, at least in part, on the one or more determined respiration states of the patient. As described above, the electrical stimulation provided to the patients may be based on one or more stimulation waveforms (SW) generated by the stimulation waveform generator 860. Various characteristics of the stimulation waveform (e.g., amplitude, phase, duty cycle, frequency, pulse-widths, and so on) may be based on the patient's respiratory state.

The calibration mode may be used to calibrate the device 800 to a particular patient and to update various operating parameters of the device 800 based on changes in the patient's physical condition, physiological condition, sleep state, level of consciousness, and/or other suitable factors. More specifically, when the device 800 operates in the calibration mode, the control circuit 820 may receive signals from the contacts 810, the sensors 840, and/or the external sensors 845, provide electrical stimulation to one or more portions of the patient's oral cavity, and then receive updated signals from the contacts 810, the sensors 840, and/or the external sensors 845 to measure the results or effectiveness of the electrical stimulation provided to the patient. The results or effectiveness of the electrical stimulation may be used to identify one or more physical or physiological attributes unique to the particular patient at a specific time, and then in response thereto adjust the electrical stimulation provided to the patient.

The compliance mode may be used to determine a level of compliance with which the patient uses the device 800. More specifically, when the device 800 operates in the compliance mode, the control circuit 820 may receive signals (S1) from contacts 810 that indicate one or more states of the patient's upper airway. At least one of these states can be an impedance level between at least two of the contacts 810. Because the level of impedance is relatively low (e.g., less than a value) when the device 800 is properly positioned within the patient's oral cavity and the level of impedance is relatively high (e.g., greater than a value) when the device 800 is not properly positioned within the patient's oral cavity, the level of impedance between the at least two of the contacts 810 can be used to determine whether device 800 is properly positioned within the patient's oral cavity, and therefore indicate whether the patient is in compliance with a prescribed use of the device 800.

The processor 870 may also include a digital signal processor (DSP) 872. Although the DSP 872 is depicted as part of the processor 870, for other implementations, the DSP 872 can be separate from and coupled to the processor 870. In some aspects, the DSP 872 may be used convert signals from the time domain to the frequency domain, for example, using a suitable Fast Fourier Transfer (FFT) function. After a particular signal is converted to the frequency domain, the DSP 872 can analyze frequency components of the converted signal to predict the onset of disordered breathing and/or to detect a presence of disordered breathing in the patient.

To more fully understand the example embodiments, an example relationship between a patient's respiration state and one or more signals provided by contacts 810(1)-810(2) is first described. When a patient falls asleep, the patient's tongue and diaphragm are typically the last muscles to receive activation signals (e.g., the last muscles that experience a reduction in muscle tone); conversely, when the patient wakes up, the patient's tongue and diaphragm are typically the first muscles to regain muscle tone (e.g., based on a return of the patient to an awake state. As a result, one or more states of the patient's upper airway may be may be monitored to determine and/or predict when the patient is falling asleep and when the patient is waking up (e.g., to determine a level or depth of sleep). This may be of enormous significance for surgical procedures during which a patient is to be rendered unconscious by the application of general anesthetics. For one example, the ability to accurately determine whether the patient is sufficiently asleep to begin surgery may reduce the chances of the patient prematurely waking up and/or may prevent an over-application of general anesthetics to the patient. For another example, the ability to accurately determine whether the patient is waking up and/or to predict when the patient is about to wake up from the effects of the general anesthetics may allow an anesthesiologist extra time to administer additional general anesthetics if the surgery is not complete.

FIG. 9A shows an example signal 900A indicating a normal (e.g., stable) breathing pattern of a patient that is asleep during five inspiration and expiration phases 910(1)-910(5) and 911(1)-911(5), respectively. The signal 900A may be provided by contacts 810(1)-810(2) of device 800, for example, in the manner described above with respect to FIGS. 8A-8B. The signal 900A is at a relatively low or minimum value 901 at points between the patient's expiration phases 911 and the patient's inspiration phases 910. For example, time t₀ corresponds to a point between the patient's previous expiration phase (not shown for simplicity) and the first inspiration phase 910(1). Just before the patient begins the first inspiration phase 910(1) at time t₁, the signal 900A begins increasing in magnitude at a first, relatively low rate. The relatively low positive slope of the signal 900A may indicate a stiffening of the tongue and/or activation of the Palatoglossus muscle in preparation for inspiration.

As the patient continues inspiring at time t₂, the signal 900A increases in magnitude at a second, relatively high rate (e.g., as compared to the first, relatively low rate between times t₁ and t₂). The relatively high positive slope of the signal 900A between times t₂ and t₃ may indicate an increased stiffening of the tongue and/or an increased activation of the Palatoglossus muscle, for example, to maximize upper airway patency during inspiration. At the peak of the patient's first inspiration phase 910(1), at time t₃, the signal 900A reaches a relatively high or maximum level 902, slightly decreases at the end of the first inspiration phase 910(1) (at time t₄), and then settles at a plateau level 903 between times t₄ and t₅. The time period between times t₄ and t₅ may be referred to herein as a “dwell period.”

At time t₅, the patient enters the first expiration phase 911(1). In response thereto, the signal 900A rapidly increases back to the maximum level 902 at time t₆. Then, as the patient exhales, the signal 900A decreases at a first, relatively high rate between times t₆ and t₇, and then decreases at a second, relatively low rate between times t₇ and t₈ (e.g., as compared to the first, relatively high rate between times t₆ and t₇). The relatively high negative slope of the signal 900A between times t₆ and t₇ may indicate a rapid relaxing of the tongue (and/or other oral musculature) during which a significant portion of the air is released from the patient's lungs. The relatively low negative slope of the signal 900A between times t₇ and t₈ may indicate a rapid slowing of the relaxing of the tongue (and/or other oral musculature), which in turn may indicate a transition between the first expiration phase 911(1) and the second inspiration phase 910(2). At time t₈, the signal 900A reaches a trough T1A at or near the minimum level 901.

As described above, the signal 900A begins increasing in magnitude prior to each inspiration phase 910 (e.g., between times t₀ and t₁), which may be caused by the tongue flexing, stiffening, and/or moving forward (e.g., in an anterior direction) to increase upper airway patency during the inspiration phases 910(1)-910(5). The movement of the tongue prior to each inspiration phase 910 may also be caused by negative pressure in the patient's oral cavity. As a result, the example embodiments may be able to accurately predict the onset of each inspiration phase 910 based at least in part on an increase in magnitude of signal 900A relative to the minimum level 901. Applicant notes that the time period between the peak of the signal 900A during the first inspiration phase 910(1) (e.g., the first peak P1A at time t₃) and the peak of the signal 900A during the first expiration phase 911(1) (e.g., the second peak P2A at time t₆) is a relatively constant value for patients breathing normally during sleep.

As depicted in the example of FIG. 9A, the peaks of signal 900A corresponding to the inspiration phases 910 and the expiration phases 911 of the patient's respiration cycle are relatively constant. More specifically, the peaks P1A and P2A corresponding to the first inspiration phase 910 and the first expiration phase 911 are not only similar in shape to each other, but are also similar in shape to the peaks P3A and P4A corresponding to the second inspiration phase 910 and the second expiration phase 920 of the patient, are similar in shape to the peaks P5A and P6A corresponding to the third inspiration phase 910 and the third expiration phase 911 of the patient, and so on.

As described above with respect to FIG. 9A, the signal 900A depicts a full respiratory cycle of the patient starting with the onset of a first inspiration phase 910(1) at time to and ending with the conclusion of the first expiration phase 911(1) at time t₈. The duration of the full respiratory cycle may be calculated by finding the time difference T_{cycle} between times to and t₈. This duration per breath may be used to calculate real-time respiration rate on the standard breaths per minute basis. Any suitable statistical calculations may be used to improve the reliability of the respiration rate calculation and/or ensure that intermittent breathing anomalies have a minimal impact upon the calculated respiration rate. Examples of these calculations may include, but are not limited to, moving averages, weighted moving averages, and exponential smoothing. In addition, the shape of signal 900A during successive inspiration and expiration phases 910 and 920 (e.g., during successive respiratory cycles) remains relatively constant as a function of time, for example, thereby indicating normal breathing of the patient.

FIG. 9B shows an example signal 900B indicating a disordered breathing state 920, followed by an arousal state 921, followed by a normal breathing state 922 of an example patient. The signal 900B may be provided by contacts 810(1)-810(2) of device 800, for example, in the manner described above with respect to FIGS. 8A-8B. As depicted in FIG. 9B, when the patient is in the disordered breathing state 920, the signal 900B is less periodic and exhibits greater variations in peak magnitudes than the example signal 900A of FIG. 9A. In addition, the signal 900B does not exhibit the plateaus or “dwell times” associated with the example signal 900A (e.g., between times t₄ and t₅ depicted in FIG. 9A).

More specifically, although the first peak P1B of signal 900B reaches the maximum level 902, subsequent peaks P3B and P5B of signal 900B do not reach the maximum level 902 determined for the normal breathing pattern of the patient (e.g., as depicted by signal 900A in FIG. 9A). Thus, in some aspects, the control circuit 820 may predict or detect the onset of disordered breathing based on peaks of the signal provided by contacts 810 (e.g., peaks P3B and P5B of signal 900B) decreasing in magnitude (by more than a first value V1) over a time period and/or falling below the maximum level 902 by more than a second value V2. For some implementations, the time period may be based on the respiration period of the patient. For example, in some aspects, the time period may be approximately equal to the duration of a normal respiration period of the patient. In other aspects, the time period may be approximately equal to a number N of normal respiration periods of the patient (e.g., where N is an integer greater than 1). For other implementations, the time period may be a dynamic value that can be adjusted by the control circuit 820 based on sensing data previously obtained from the patient (e.g., based on historical sleep data of the patient stored in memory such as data store 889 of memory 880). Alternatively, the time period may be a static value.

In addition, the first trough T1B of signal 900B falls to a third value V3 that is less than the minimum value 901 determined for the normal breathing pattern of the patient (e.g., as depicted by signal 900A in FIG. 9A). Thus, in other aspects, the onset of the disordered breathing state 920 may be predicted or detected based on a magnitude of the signal 900B falling below the minimum level 901 by an amount (e.g., at trough T1B of signal 900B). Further, it is noted that the signal 900B of FIG. 9B is less periodic than the signal 900A of FIG. 9A. Thus, in other aspects, the control circuit 820 may predict the onset of the disordered breathing state 920 based on a decrease in periodicity of the signal provided by contacts 810, for example, as compared to the signal 900A depicted in FIG. 9A.

At time t₁ in the example of FIG. 9B, the magnitude of signal 900B rapidly increases and reaches a peak P6B that is significantly greater than the maximum level 902 (e.g., by a value V4). This sudden and rapid increase in the magnitude of signal 900B may indicate an arousal state 921 of the patient. The arousal state 921 may also be indicated by a second peak P7B exceeding the maximum level 902 (e.g., by a value V5) and/or by troughs T6B and T7B of signal 900B being significantly less than the minimum level 901 (e.g., by values V6 and V7, respectively). Thereafter, at time t₂ in the example of FIG. 9B, the signal 900B begins to resemble the signal 900A of FIG. 9A, thereby indicating a beginning of a normal breathing state 922 of the patient.

As discussed above, for at least some implementations, the device 800 may predict or detect the onset of disordered breathing in a patient by comparing signal 900B with one or more reference signals indicative of normal breathing of the patient. For example, signal 900A of FIG. 9A may be indicative of the patient's normal breathing patterns, and may be stored in a memory of device 800 (e.g., in data store 889 of memory 880 of FIG. 8A). Thus, in some aspects, device 800 may predict or detect the onset of disordered breathing by comparing signal 900B (e.g., indicating a present state of the patient's upper airway) with signal 900A (e.g., indicating a previous normal breathing state of the patient's upper airway).

FIG. 9C shows an example signal 900C indicating an occurrence of snoring while the patient is asleep. Applicant believes that snoring results from a reduction in the muscle tone of the upper airway during the inspiration phase of breathing during sleep. Specifically, this reduction in muscle tone during sleep may allow tissue within and/or associated with the patient's upper airway to vibrate during inspiration, which in turn creates the snoring noise. Although disruptive (particularly to a spouse sleeping next to the patient), snoring may present significant risks to the patient including, for example, loss of sleep, hypoxemia, and possibly suffocation. Thus, detecting the onset of snoring may be an important tool to reduce the occurrence and magnitude of snoring (or to even prevent snoring altogether).

The signal 900C may be provided by contacts 810(1)-810(2) of device 800, for example, in the manner described above with respect to FIGS. 8A-8B. Similar to the signal 900A of FIG. 9A, the signal 900C of FIG. 9C exhibits a relatively constant periodicity. However, in contrast to the signal 900A of FIG. 9A, the signal 900C of FIG. 9C reaches a first peak P1C having a first magnitude 931 during the inspiration phase 910 much faster than the signal 900A of FIG. 9A, drops off to a level L2 having a second magnitude 932, and then slowly increases to a second peak P3C corresponding to the maximum level 902. As shown in FIG. 9C, the first peak P1C of signal 900C occurs during the inspiration phase 910 of the patient's first respiration period T1, and the second peak P2C of signal 900A occurs during the expiration phase 911 of the patient's first respiration period T1. The first magnitude value 931 is less than the maximum level 902 by a first threshold amount (THR₁), and the second magnitude value 932 is less than the first magnitude value 931 by a second threshold amount (THR₂).

Thus, in some aspects, the onset of snoring may be predicted or detected based on a determination that during a given respiration cycle of the patient, a first peak of the signal provided by contacts 810 is less than a second peak of the signal by more than a threshold amount THR₁. More specifically, for the example of FIG. 9C, if a first peak P1C of the signal 900C is less than a second peak P2C of the signal 900C by the threshold amount THR₁, then the control circuit 820 may indicate the onset of snoring.

In addition, when the patient is snoring, the signal 900C may not reach the maximum level 902 during the inspiration phase 910, and may not plateau at level 903 between the inspiration phase 910 and the expiration phase 911 (e.g., as compared with the example signal 900A of FIG. 9A). Thus, in some aspects, the onset of snoring may be predicted or detected based on a determination that during a given respiration cycle of the patient, a first peak of the signal provided by contacts 810 is less than maximum level 902 and/or that the signal does not exhibit a plateau between inspiration and expiration phases of the patient's respiration cycle. More specifically, for the example of FIG. 9C, if the first peak P1C of the signal 900C is less than the maximum level 902 by the threshold amount THR₁ and/or if the signal 900C does not have a relatively constant magnitude between times t₄ and t₅, then the control circuit 820 may indicate the onset of snoring.

For other implementations, the device 800 may predict or detect the onset of snoring in a patient by comparing signal 900C with one or more reference signals indicative of normal breathing of the patient. For example, signal 900A of FIG. 9A may be indicative of the patient's normal breathing patterns, and may be stored in a memory of device 800 (e.g., in data store 889 of memory 880 of FIG. 8A). Thus, in some aspects, device 800 may predict or detect the onset of snoring by comparing signal 900C (e.g., indicating a present state of the patient's upper airway) with signal 900A (e.g., indicating a previous normal breathing state of the patient's upper airway).

FIG. 9D shows an example signal 900D indicating a patient experiencing apnea (e.g., obstructive sleep apnea) during sleep. The signal 900D may be provided by contacts 810(1)-810(2) of device 800, for example, in the manner described above with respect to FIGS. 8A-8B. During the inspiration phase 910 (which begins prior to time t₁), the magnitude of the signal 900D increases until its peak value P1D is approximately equal to the maximum level 902. Then, during the expiration phase 911, the magnitude of signal 900D rapidly decreases from the peak P1D at or near the maximum level 902 to a trough T1D at or near a depressed level 905 during a relatively short time period T2. The change in magnitude of signal 900D between peak P1D and trough T1D is depicted in FIG. 9D as an apnea threshold amount THRₐₚₙₑₐ.

Referring also to FIG. 9A, the negative slope of signal 900E at or near time t₄ is much greater than the negative slope of signal 900A (e.g., by more than a threshold slope value (THRₛₗₒₚₑ). Thus, in some aspects, the onset of apnea may be predicted or detected based on the magnitude of the signal provided by contacts 810 decreasing by more than an amount during a time period. More specifically, for the example of FIG. 9D, the onset of apnea may be predicted or detected based on the magnitude of signal 900D decreasing by the apnea threshold amount THRₐₚₙₑₐ during time period T2. In some aspects, the value of the apnea threshold amount THRₐₚₙₑₐ may be dynamically adjusted based on a number of factors specific to a particular patient.

It is noted that the depressed level 905 may be less than the minimum level 901 corresponding to signal 900A associated with normal breathing, as described above with respect to FIG. 9A. Referring also to FIG. 9A, the magnitude of signal 900E at trough T1D is much less than the magnitude of signal 900A at first trough T1A. Thus, in other aspects, the onset of apnea may be predicted or detected based on the magnitude of the signal provided by contacts 810 reaching a value that is more than a threshold amount THR₃ less than the minimum level 901. More specifically, for the example of FIG. 9D, the onset of apnea may be predicted or detected based on the magnitude of signal 900D reaching a level that is less than the minimum level 901 by the apnea threshold amount THRₐₚₙₑₐ.

It is also noted that signal 900D does not exhibit a plateau during the dwell time between the inspiration phase 910 and the expiration phase 911 of the patient's respiration cycle. Thus, in other aspects, the onset of the apnea state 920 may be predicted or detected based on an absence of a plateau in signal 900D during the dwell time between the inspiration phase 910 and the expiration phase 911 of the patient's respiration cycle.

For other implementations, the device 800 may predict or detect the onset of apnea in a patient by comparing signal 900D with one or more reference signals indicative of normal breathing of the patient. For example, signal 900A of FIG. 9A may be indicative of the patient's normal breathing patterns, and may be stored in a memory of device 800 (e.g., in data store 889 of memory 880 of FIG. 8A). Thus, in some aspects, device 800 may predict or detect the onset of apnea by comparing signal 900D (e.g., indicating a present state of the patient's upper airway) with signal 900A (e.g., indicating a previous normal breathing state of the patient's upper airway).

FIG. 9E shows an example signal 900E indicating a patient experiencing CNS depression. The signal 900E may be provided by contacts 810(1)-810(2) of device 800, for example, in the manner described above with respect to FIGS. 8A-8B. As depicted in FIG. 9E, when the patient is in the disordered breathing state 920, the signal 900E is less periodic and exhibits greater variations in peak magnitudes than the example signal 900A of FIG. 9A. In addition, the signal 900E does not exhibit the plateaus or “dwell times” associated with the example signal 900A (e.g., between times t₄ and t₅ depicted in FIG. 9A). Thus, the control circuit 820 may predict or detect the onset of disordered breathing in the manner described above with respect to FIG. 9B.

By time t₁ in the example of FIG. 9E, the magnitude of signal 900E reaches a minimum value 901 and remains relatively constant at the minimum value 901 for a duration of time between times t₁ and t₂. In other words, the signal 900E flat-lines at time t₁, thereby indicating little or no respiration effort in the patient (e.g., the patient may have stopped breathing due to CNS depression). Thus, in some aspects, the onset of CNS depression may be predicted or detected based on the magnitude of the signal provided by contacts 810 flat-lining (e.g., remaining at a relatively constant level for more than a time period). More specifically, for the example of FIG. 9E, the onset of CNS depression may be predicted or detected based on the magnitude of signal 900E remaining at the minimum level 901 for more than a time period (T_{FL}), for example, depicted in the example of FIG. 9E as the duration of time between time t₁ and a time t_{X}. In some aspects, the time period T_{FL} may be substantially equal to a duration of the patient's normal inspiration phase 910 or substantially equal to a duration of the patient's normal expiration phase 911.

Thereafter, at time t₂ in the example of FIG. 9E, the signal 900E begins to resemble the signal 900A of FIG. 9A, thereby indicating a beginning of a normal breathing state 922 of the patient. Note that for the example of FIG. 9E, the signal 900E does not exhibit a spike in magnitude when the patient returns to a normal breathing state 922. This is in contrast to the spike in magnitude of signal 900B of FIG. 9B associated with an arousal of the patient following a disordered breathing state 920 (e.g., between times t₁ and t₂ in FIG. 9B). As a result, it may be possible to distinguish between disordered breathing resulting from a breathing obstruction and disordered breathing resulting from CNS depression by analyzing signals provided by contacts 810.

More specifically, in some aspects, the presence of one or more spikes in magnitude of signals provided by contacts 810 (as depicted by the peaks P6B and P7B in the signal 900B of FIG. 9B) may indicate that the disordered breathing results from an obstruction (e.g., a type of apnea), while the presence of a flat-lining of signals provided by contacts 810 (as depicted by the magnitude of the signal 900E remaining at a relatively constant level for more than a time period) may indicate that the disordered breathing results from CNS.

For other implementations, the device 800 may predict or detect the onset of CNS depression in a patient by comparing signal 900E with one or more reference signals indicative of normal breathing of the patient. For example, signal 900A of FIG. 9A may be indicative of the patient's normal breathing patterns, and may be stored in a memory of device 800 (e.g., in data store 889 of memory 880 of FIG. 8A). Thus, in some aspects, device 800 may predict or detect the onset of CNS depression by comparing signal 900E (e.g., indicating a present state of the patient's upper airway) with signal 900A (e.g., indicating a previous normal breathing state of the patient's upper airway).

As discussed above, for at least some implementations, the control circuit 820 of device 800 may combine, supplement, or verify information contained in signals S1 provided by contacts 810 with information contained in signals S2 provided by sensors 840 and/or with information contained in signals S3 provided by external sensors 845. The information contained in signals S1 provided by contacts 810 may indicate a state of the patient's upper airway (e.g., based on electrical activity in the musculature, nerves, and tissue within or connected to the patient's upper airway). The information contained in signals S2 provided by sensors 840 may indicate movement of the patient's upper airway, sounds of the patient's upper airway, airflow in the patient's upper airway, an oxygenation rate of the patient, a level of carbon dioxide of the patient, a heartrate of the patient, charging and discharging times of the patient's tongue, or any other suitable indications of the patient's respiration. The information contained in signals S3 provided by external sensors 845 may indicate movement of the patient's chest (e.g., indicating volume changes in response to inspiration and expiration of the patient), movement of the patient's abdomen, and/or movement of the patient's jaw.

A number of comparisons between waveforms of signals provided by the contacts 810 and waveforms of signals indicative of sounds of the patient's upper airway, airflow in the patient's upper airway, an oxygenation rate of the patient, a heartrate of the patient, movement of the patient's chest, and movement of the patient's abdomen are described below with respect to FIGS. 10A-10D. As used herein with respect to FIGS. 10A-10D, the term “apnea” refers to a drop in airflow amplitude ≧90% of the patient's baseline value that lasts ten seconds or longer, and for which at least 90% of the event duration meets the amplitude reduction criterion. The term “obstructive apnea” refers to a breathing disorder characterized by brief interruptions of breathing during sleep. In obstructive apnea, the muscles of the soft palate around the base of the tongue and the uvular relax, obstructing the airway. Obstructive sleep apnea is characterized by the presence of respiratory efforts (abdomen/chest band activity is present). The term “central apnea” refers to a breathing disorder characterized by brief interruptions of breathing during sleep. Central apnea occurs when the brain fails to send the appropriate signals to the breathing muscles to initiate respiration; hence, central apnea is characterized by a lack of respiratory effort (abdomen/chest band activity is not present). The term “mixed apnea” refers to a breathing disorder characterized by brief interruptions of breathing during sleep. Mixed sleep apnea consists of both central and obstructive sleep apnea.

Further, the term “snoring” refers to sounds emanating from the patient's upper airway louder than 60 dB and having a duration between 0.25 and 5 seconds, and the term “Respiratory Effort Related Arousal (RERA)” refers to breaths lasting at least 10 seconds and characterized by increasing respiratory effort or flattening of the nasal cannula pressure that results in an arousal from sleep when the sequence of breaths does not meet the criteria for either an apnea or a hypopnea. In some aspects, a RERA may be considered to be a milder form of sleep disordered breathing than either apnea or hypopnea.

For example, FIG. 10A shows a graph 1000A depicting a number of respiration, physical, and physiological attributes of a patient experiencing normal breathing (e.g., there is not a presence of disordered breathing, apnea, CNS, or other respiratory difficulties in the patient). The graph 1000A includes an audio waveform, an airflow waveform (W1A), a thermistor airflow waveform (W2A), a chest movement waveform (W3A), an abdomen movement waveform (W4A), an oxygenation rate (Sp0₂) waveform, a heartrate waveform, and a sum waveform (W_{sumA}). The audio waveform, which may be generated by a suitable microphone either attached to device 800 or coupled to device 800, records audio signals of the patient (e.g., loudness of breath and snoring). Each of the airflow waveform W1A and the thermistor airflow waveform W2A indicates an amount of airflow through the patient's upper airway. The chest movement waveform W3A indicates an amount of movement in the patient's chest (e.g., expansion and compression of the chest caused by inspiration and expiration phases, respectively, of the patient). The abdomen movement waveform W4A indicates an amount of movement in the patient's abdomen (e.g., movement due to inspiration and expiration phases of the patient). The sum waveform W_{sumA} may indicate a sum of the aforementioned waveforms W1A-W4A.

For the example graph 1000A, the audio waveform, the airflow waveform W1A, the thermistor airflow waveform W2A, the chest movement waveform W3A, the abdomen movement waveform W4A, the Sp0₂ waveform, the heartrate waveform, and the sum waveform W_{sumA} were provided by the Medibyte® device while connected to a test patient. Of course, for other implementations, the airflow waveform W1A and the thermistor airflow waveform W2A may be provided by sensors 840 of device 800, and the chest movement waveform W3A and the abdomen movement waveform W4A may be provided by external sensors 845 coupled to device 800. In some aspects, the waveforms W1A-W4A may be generated by a pressure airflow sensor, a thermistor airflow sensor, a chest band, and an abdomen band, respectively.

The graph 1000A also includes a signal S1A provided by contacts 810 of the device 800. The signal S1A, which may be one implementation of the signal 900A of FIG. 9A, may be an EMG signal indicative of electrical activity of the musculature, nerves, and/or tissue within or associated with the patient's upper airway. Thus, for at least some implementations, the signal S1A may be indicative of the state of the patient's upper airway.

As depicted in FIG. 10A, each of the waveforms W1A-W4A exhibits a substantially constant shape and periodicity (e.g., similar to the signal 900A depicted in FIG. 9A), and indicates a normal breathing of the patient. The signal S1A provided by contacts 810 also exhibits a substantially constant shape and periodicity. For some implementations, the state of the patient's upper airway may be monitored by one or more of the waveforms W1A-W4A and/or by the signal S1A. For other implementations, the state of the patient's upper airway may be monitored by the signal S1A and then supplemented or verified by one or more of the waveforms W1A-W4A and/or. In some aspects, the airflow waveforms W1A-W2A can be correlated with the signal S1A, for example, to verify indications of disordered breathing derived from an analysis of the signal S1A. In other aspects, the airflow waveforms W1A-W2A can be used as indications of disordered breathing while the contacts 810 of the device 800 provide electrical stimulation to one or more portions of the patient's oral cavity.

More specifically, referring also to FIG. 8B, the mode control circuit 875 may assert the mode signal to a first state that causes device 800 to operate in the sensing mode, for example, so that contacts 810 can provide signals such as signal S1A to the control circuit 820. As discussed above, when device 800 operates in the sensing mode, the control circuit 820 may analyze signals 51 provided by the contacts 810 to determine a state of the patient's upper airway, to predict or detect the onset of disordered breathing, to determine a level of consciousness of the patient, to determine a sleep level of the patient, and/or to determine a respiration state of the patient.

Thereafter, the mode control circuit 875 may assert the mode signal to a second state that causes device 800 to operate in the therapy mode, for example, so that the control circuit 820 can provide, via stimulation waveform generator 860, stimulation waveforms that cause the contacts 810 to provide one or more patterns of electrical stimulation to suitable portions of the patient's oral cavity. As discussed above, the one or more patterns of electrical stimulation provided to the patient's oral cavity may be configured based on a monitored state of the patient's upper airway and/or one or more physiological conditions indicated by signals provided by sensors 840 and external sensors 845. In some aspects, the sensors 840 and external sensors 845 may continue providing respective signals S2 and S3 to the control circuit 820 during the therapy mode. In other aspects, other sensors (e.g., such as those associated with the aforementioned Medibyte® device) may continue providing the waveforms depicted in FIGS. 10A-10D to the control circuit 820 during the therapy mode. In this manner, the control circuit 820 may continue receiving indications of the patient's respiratory state even when the device 800 toggles between the sensing mode and the therapy mode.

FIG. 10B shows a graph 1000B depicting a number of respiration, physical, and physiological attributes of a patient snoring while asleep. The graph 1000B includes an audio waveform, an airflow waveform (W1B), a thermistor airflow waveform (W2B), a chest movement waveform (W3B), an abdomen movement waveform (W4B), an oxygenation rate (Sp0₂) waveform, a heartrate waveform, and a sum waveform (W_{sumB}). The waveforms depicted in FIG. 10B may be provided to the control circuit 820 in a manner similar to that described above with respect to FIG. 10A. The graph 1000B also includes a signal S1B provided by contacts 810 of the device 800. The signal S1B, which may be one implementation of the signal 900C of FIG. 9C, may be an EMG signal indicative of electrical activity of the musculature, nerves, and/or tissue within or associated with the patient's upper airway. Thus, for at least some implementations, the signal S1B may be indicative of the state of the patient's upper airway. As discussed above, Applicant believes that snoring results from a reduction in the muscle tone of the upper airway during the inspiration phase of breathing during sleep.

FIG. 10C shows a graph 1000C depicting a number of respiration, physical, and physiological attributes of a patient experiencing disordered breathing such as hypopnea. The graph 1000C includes an audio waveform, an airflow waveform (W1C), a thermistor airflow waveform (W2C), a chest movement waveform (W3C), an abdomen movement waveform (W4C), an oxygenation rate (Sp0₂) waveform, a heartrate waveform, and a sum waveform (W4C). The waveforms depicted in FIG. 10C may be provided to the control circuit 820 in a manner similar to that described above with respect to FIG. 10A. The graph 1000C also includes a signal S1C provided by contacts 810 of the device 800. The signal S1C, which may be one implementation of the signal 900D of FIG. 9D, may be an EMG signal indicative of electrical activity of the musculature, nerves, and/or tissue within or associated with the patient's upper airway. Thus, for at least some implementations, the signal S1C may be indicative of the state of the patient's upper airway.

During a first inspiration phase 910(1), the magnitude of the waveform W1C initially increases at a relatively low rate, and then rapidly increases to a maximum level, for example, as compared with the inspiration phases 910 of waveform 900A in FIG. 10A. In some aspects, during a first portion of the first inspiration phase 910(1), the positive slope of waveform W1C is less than the positive slope of waveform W1A of FIG. 10A, and during a second portion of the first inspiration phase 910(1), the positive slope of waveform W1C is greater than the positive slope of waveform W1A of FIG. 10A. Then, duration the first expiration phase 911(1), the magnitude of the waveform W1C decreases at a relatively high rate, for example, as compared with the expiration phases 911 of waveform 900A in FIG. 10A. In some aspects, the negative slope of the signal W1C during the first expiration phase 911(1) is greater than the negative slope of the signal W1A during the expiration phases 911 of FIG. 10A.

During the second inspiration phase 910(2), the magnitude of the waveform W1C initially increases at a relatively low rate, and then rapidly increases to a maximum level, for example, as compared with the inspiration phases 910 of waveform 900A in FIG. 10A. In some aspects, during a first portion of the second inspiration phase 910(2), the positive slope of waveform W1C is less than the positive slope of waveform W1A of FIG. 10A, and during a second portion of the second inspiration phase 910(1), the positive slope of waveform W1C is greater than the positive slope of waveform W1A of FIG. 10A. Then, duration the second expiration phase 911(2), the magnitude of the waveform W1C decreases at a relatively high rate, for example, as compared with the expiration phases 911 of waveform 900A in FIG. 10A. In some aspects, the negative slope of the signal W1C during the second expiration phase 911(2) is greater than the negative slope of the signal W1A during the expiration phases 911 of FIG. 10A.

During the third inspiration phase 910(3), the magnitude of the waveform W1C initially increases at a relatively low rate, and then rapidly increases to a maximum level, for example, as compared with the inspiration phases 910 of the waveform in FIG. 10A. In some aspects, during a first portion of the third inspiration phase 910(3), the positive slope of waveform W1C is less than the positive slope of waveform W1A of FIG. 10A, and during a second portion of the third inspiration phase 910(3), the positive slope of waveform W1D is greater than the positive slope of waveform W1A of FIG. 10A. Then, duration the third expiration phase 911(3), the magnitude of the waveform W1C decreases at a relatively high rate, for example, as compared with the expiration phases 911 of the waveform in FIG. 10A. In some aspects, the negative slope of the signal W1C during the third expiration phase 911(3) is greater than the negative slope of the signal W1A during the expiration phases 911 of FIG. 10A.

Applicant also notes that the inspiration phases 910 of the waveform W1C increase in duration as a function of time, while the expiration phase 911 of the waveform W1C decrease in duration as a function of time. For example, the duration D3, of the third inspiration phase 910(3) is greater than the duration D2, of the second inspiration phase 910(2), and the duration D2, of the second inspiration phase 910(2) is greater than the duration D1, of the first inspiration phase 910(1). Conversely, the duration D3, of the third expiration phase 911(3) is less than the duration D2ₑ of the second expiration phase 911(2), and the duration D2ₑ of the second expiration phase 911(2) is less than the duration D1ₑ of the first expiration phase 911(1).

Thus, the control circuit 820 may detect an onset of hypopnea based on one or more of the following: successive inspiration phases 910 increasing in duration and successive expiration phases 911 decreasing in duration (e.g., as a function of time); the positive slope of waveform W1C decreasing in magnitude during successive inspiration phases 910 and the negative slope of waveform W1C decreasing in magnitude during successive expiration phases 911; the positive slope of waveform W1C during a first portion of inspiration phases 910 being less than the positive slope of waveform W1A during a first portion of inspiration phases 910 and the positive of waveform W1C during a second portion of inspiration phases 910 being greater than the positive slope of waveform W1A during a second portion of inspiration phases 910; or the negative slope of the signal W1C during expiration phases 911 being greater than the negative slope of the signal W1A during expiration phases 911 of FIG. 10A.

After the third expiration phase 911(3), the magnitude of the waveform W1C remains relatively constant for a duration D4 that is greater than a normal breathing cycle or period of the patient. More specifically, between times t₁ and t₂ in the graph 1000C, the magnitude of the waveform W1C increases by less than a threshold value, and the magnitude of the waveform W2C decreases. Thus, the control circuit 820 may detect an occurrence of hypopnea based, at least in part, on the magnitude of the waveform W1C remaining relatively constant for a time period greater than the duration of the patient's normal breathing period.

Just after time t₂, the signal S1C provided by the contacts 810 spikes in magnitude, which may indicate an arousal of the patient and a return to normal breathing. For the example of FIG. 10C, the waveform W1C returns to a shape and periodicity associated with normal breathing for a duration between times t₂ and t₃ (e.g., as may be correlated to the shape and periodicity of the waveform W1A of FIG. 10A being indicative of normal breathing).

Then, just after time t₃, the waveform W1C returns to a shape and periodicity associated a hypopnea state, for example, as indicated by the shape and periodicity of waveform W1C between times t₁ and t₂. More specifically, after time t₃, the magnitude of the waveform W1C remains relatively constant for a duration D5 that is greater than a normal breathing cycle or period of the patient. More specifically, between times t₃ and t₄ in the graph 1000C, the magnitude of the waveform W1C increases by less than a threshold value. Thus, the control circuit 820 may detect an occurrence of hypopnea based, at least in part, on the magnitude of the waveform W1C remaining relatively constant for a time period greater than the duration of the patient's normal breathing period.

At or around time t₄, the signal S1C provided by the contacts 810 spikes in magnitude, which may indicate an arousal of the patient and a return to normal breathing. At time t₅, the waveform W1C returns to a shape and periodicity associated with normal breathing (e.g., as may be correlated to the shape and periodicity of the waveform W1A of FIG. 10A being indicative of normal breathing). Thus, for some implementations, the control circuit 820 may detect an arousal of the patient and predict a return to normal breathing based on a sudden spike in magnitude of the signal S1C, and may verify the return to normal breathing based on the waveform W1C increasing in magnitude consistent with inspiration phases 910 of normal breathing (e.g., as may be derived from the graph 1000A of FIG. 10A).

FIG. 10D shows a graph 1000D depicting a number of respiration, physical, and physiologic attributes of a patient experiencing disordered breathing such as obstructive sleep apnea (OSA). The graph 1000D includes an audio waveform, an airflow waveform (W1D), a thermistor airflow waveform (W2D), a chest movement waveform (W3D), an abdomen movement waveform (W4D), an oxygenation rate (Sp0₂) waveform, a heartrate waveform, and a sum waveform (W_{sumD}). The waveforms depicted in FIG. 10D may be provided to the control circuit 820 in a manner similar to that described above with respect to FIG. 10A. The graph 1000D also includes a signal S1D provided by contacts 810 of the device 800. The signal S1D, which may be one implementation of the signal 900E of FIG. 9E, may be an EMG signal indicative of electrical activity of the musculature, nerves, and/or tissue within or associated with the patient's upper airway. Thus, for at least some implementations, the signal S1D may be indicative of the state of the patient's upper airway.

During a first inspiration phase 910(1), the magnitude of the waveform W1D initially increases at a relatively low rate, and then rapidly increases to a maximum level, for example, as compared with the inspiration phases 910 of the waveform in FIG. 10A. In some aspects, during a first portion of the first inspiration phase 910(1), the positive slope of waveform W1D is less than the positive slope of waveform W1A of FIG. 10A, and during a second portion of the first inspiration phase 910(1), the positive slope of waveform W1D is greater than the positive slope of waveform W1A of FIG. 10A. Then, duration the first expiration phase 911(1), the magnitude of the waveform W1D decreases at a relatively high rate, for example, as compared with the expiration phases 911 of the waveform in FIG. 10A. In some aspects, the negative slope of the signal W1D during the first expiration phase 911(1) is greater than the negative slope of the signal W1A during the expiration phases 911 of FIG. 10A.

During the second inspiration phase 910(2), the magnitude of the waveform W1D initially increases at a relatively low rate, and then rapidly increases to a maximum level, for example, as compared with the inspiration phases 910 of the waveform in FIG. 10A. In some aspects, during a first portion of the second inspiration phase 910(2), the positive slope of waveform W1D is less than the positive slope of waveform W1A of FIG. 10A, and during a second portion of the second inspiration phase 910(1), the positive slope of waveform W1D is greater than the positive slope of waveform W1A of FIG. 10A. Then, duration the second expiration phase 911(2), the magnitude of the waveform W1D decreases at a relatively high rate, for example, as compared with the expiration phases 911 of the waveform in FIG. 10A. In some aspects, the negative slope of the signal W1D during the second expiration phase 911(2) is greater than the negative slope of the signal W1A during the expiration phases 911 of FIG. 10A.

During the third inspiration phase 910(3), the magnitude of the waveform W1D increases at a relatively low yet constant rate, for example, as compared with the inspiration phases 910 of the waveform in FIG. 10A. In some aspects, the positive slope of waveform W1D is less than the positive slope of waveform W1A of FIG. 10A during the inspiration phases 910. Then, duration the third expiration phase 911(3), the magnitude of the waveform W1D decreases at a relatively high rate, for example, as compared with the expiration phases 911 of the waveform in FIG. 10A.

Applicant also notes that the inspiration phases 910 of the waveform W1C increase in duration as a function of time, while the expiration phase 911 of the waveform W1C decrease in duration as a function of time. For example, the duration D3ᵢ of the third inspiration phase 910(3) is greater than the duration D2ᵢ of the second inspiration phase 910(2), and the duration D2ᵢ of the second inspiration phase 910(2) is greater than the duration D1ᵢ of the first inspiration phase 910(1). Conversely, the duration D3, of the third expiration phase 911(3) is less than the duration D2ₑ of the second expiration phase 911(2), and the duration D2ₑ of the second expiration phase 911(2) is less than the duration D1ₑ of the first expiration phase 911(1).

Thus, the control circuit 820 may detect an onset of OSA based on one or more of the following: successive inspiration phases 910 increasing in duration and successive expiration phases 911 decreasing in duration (e.g., as a function of time); the positive slope of waveform W1D decreasing in magnitude during successive inspiration phases 910 and the negative slope of waveform W1D decreasing in magnitude during successive expiration phases 911; the positive slope of waveform W1D during a first portion of inspiration phases 910 being less than the positive slope of waveform W1A during a first portion of inspiration phases 910 and the positive of waveform W1D during a second portion of inspiration phases 910 being greater than the positive slope of waveform W1A during a second portion of inspiration phases 910; or the negative slope of the signal W1D during expiration phases 911 being greater than the negative slope of the signal W1A during expiration phases 911 of FIG. 10A.

After the third expiration phase 911(3), the magnitude of the waveform W1D remains relatively constant for a duration D4 that is greater than a normal breathing cycle or period of the patient. More specifically, between times t₁ and t₂ in the graph 1000D, the magnitude of the waveform W1D increases by less than a threshold value, and the magnitude of the waveform W2D decreases. Thus, the control circuit 820 may detect an occurrence of OSA based, at least in part, on the magnitude of the waveform W1D remaining relatively constant (or decreasing) for a time period greater than the duration of the patient's normal breathing period.

Thereafter, at or around time t₃, the signal S1D provided by the contacts 810 spikes in magnitude, which may indicate an arousal of the patient and a return to normal breathing. For the example of FIG. 10D, at time t₃, the waveform W1D returns to a shape and periodicity associated with normal breathing (e.g., as may be correlated to the shape and periodicity of the waveform W1A of FIG. 10A being indicative of normal breathing). Thus, for some implementations, the control circuit 820 may detect an arousal of the patient and predict a return to normal breathing based on a sudden spike in magnitude of the signal S1D, and may verify the return to normal breathing based on the waveform W1D increasing in magnitude consistent with inspiration phases 910 of normal breathing (e.g., as may be derived from the graph 1000A of FIG. 10A).

The signals S1 provided by the contacts 810 may be used to verify the indication of the patient's respiration state provided by the waveforms W1-W4, the audio signals, the heartrate signal, and/or the Sp0₂ signals depicted in FIGS. 10A-10D. In some aspects, the waveforms W1-W4, the audio signals, the heartrate signal, and/or the Sp0₂ signals may be provided by a Medibyte® device. In other aspects, the waveforms W1-W4, the audio signals, the heartrate signal, and/or the Sp0₂ signals may be provided by sensors 840 and external sensors 845 described above with respect to FIGS. 8A-8B.

For implementations in which the signals S1 provided by the contacts 810 are EMG signals, the signals S1 may provide a reference signal from which inspiratory effort may be based. More specifically, referring again to FIG. 8B, for some implementations, the control circuit 820 may combine or correlate one or more of waveforms W1-W4 (as indicators of inspiration of the patient) with the signal S1 provided by the contacts 810 (as an indication of the state of the patient's upper airway) to distinguish between a breathing obstruction and CNS depression. For one example, if one or more the waveforms W1-W4 indicate that there is no (or at least negligible) airflow in the patient and the signal S1 indicates a very low EMG level (e.g., the magnitude of the signal S1 is less than a minimum value), then the control circuit 820 may indicate the presence of CNS depression in the patient. For another example, if one or more the waveforms W1-W4 indicate that there is no (or at least negligible) airflow in the patient and the signal S1 indicates a normal EMG level (e.g., the magnitude of the signal S1 is greater than a threshold value), then the control circuit 820 may indicate the presence of a breathing obstruction in the patient.

For other implementations, a patient's nasal dilation and/or changes in the shape or tone of the patient's tongue may be used to assist in distinguishing between a breathing obstruction and CNS depressions. For one example, if there is an increase in nasal dilation and/or changes in the shape or tone of the patient's tongue—but no reduction in the patient's airflow, this may indicate the presence of a breathing obstruction. For one example, if there is not an increase in nasal dilation and there is reduction in the patient's airflow, this may indicate the presence of CNS depression. Thus, for at least some implementations, nasal dilation and/or changes in the shape or tone of the tongue may be used to distinguish between a breathing obstruction and CNS depression. In other aspects, nasal dilation and/or changes in the shape or tone of the tongue may be used, in conjunction with signals S1 provided by the contacts 810, to distinguish between a breathing obstruction and CNS depression.

In some aspects, EMG signals provided by the contacts 810 may indicate both movement and contraction of the patient's tongue, and may be able to indicate an amount of airflow in the patient's upper airway in a manner that is independent of temperature. Because output signals provided by thermistors are temperature-dependent, indications of airflow provided by contacts 810 may be used to verify or compensate thermistor readings over various temperature ranges. When EMG is too low (person in deep sleep), we can supplement the EMG with thermistor (reverse mode). Draw a state diagram to illustrate this point. Plus, a microphone can detect snoring, and then perform FFT on the waveform to determine where the snoring is coming from.

Referring again to FIG. 8B, the DSP 872 (or other suitable components of processor 870) may be used convert EMG signals provided by the contacts 810 from the time domain to the frequency domain, for example, using a suitable Fast Fourier Transfer (FFT) function, and then determine whether there is a presence of high frequency components indicative of disordered breathing. The DSP 872 may also be used to convert signals indicative of a patient's airflow (e.g., waveforms W1-W2 depicted in FIGS. 10-10D) to the frequency domain and then determine whether there is a presence of high frequency components not correlated to “normal” breathing.

FIG. 11A is an illustrative flow chart depicting an example operation 1100 for detecting and treating disordered breathing in a patient, in accordance with some embodiments. The example operation 1100 is described below with respect to device 800 of FIG. 8A for simplicity only; the example operation 1100 may be performed by other suitable device. As described above with respect to FIGS. 8A-8B, the device 800 may include at least one contact 810 adapted to make contact with a portion of an oral cavity of a patient, and may include a control circuit 820 coupled to the at least one contact 810.

First, the device 800 may operate in a first mode to detect a presence of disordered breathing in the patient based, at least in part, on a first signal received from the at least one contact 810 (1101). The control circuit 820 may detect the presence of disordered breathing based on a magnitude of the first signal varying by more than an amount during a time period (1101A), may detect the presence of disordered breathing based on a positive slope of the first signal decreasing during each of at least two successive respiratory cycles of the patient (1101B), may detect the presence of disordered breathing based on a magnitude of the first signal decreasing during a respiratory cycle of the patient (1101C), and/or may detect the presence of disordered breathing based on a combination of the first signal and a second signal received from one or more sensors 840 (1101D). The one or more sensors 840 can include at least one of a thermistor, an airflow detector, a thermocouple, or other temperature sensing devices.

In some aspects, the first signal comprises an indication of one or more states of the patient's upper airway, and the second signals comprise an indication of airflow in the patient's upper airway. In other aspects, the first signal is indicative of at least one of electrical or muscular activity in the patient's upper airway, movement in the patient's upper airway, and a change in the patient's respiration rate, and the second signals are indicative of at least one of a movement of the patient's chest, a movement of the patient's abdomen, a movement of the patient's jaw, and an airflow through the patient's upper airway.

Then, the device 800 may operate in a second mode to provide electrical stimulation via the at least one contact 810 to a portion of the patient's upper airway based on a detection of the presence of disordered breathing (1102). As described above, the device 800 can provide electrical stimulation to a portion of the patient's upper airway in a manner that may prevent the onset or reduce the severity and/or duration of the disordered breathing.

The device 800 may operate in a third mode to adjust one or more characteristics of the stimulation based on changes in the first signal resulting from a number of applications of stimulation to the patient's upper airway (1103).

FIG. 11B is an illustrative flow chart 1110 depicting an example operation for predicting an onset of apnea or disordered breathing in a patient, in accordance with some embodiments. The example operation 1100 is described below with respect to device 800 of FIG. 8A for simplicity only; the example operation 1100 may be performed by other suitable device. As described above with respect to FIGS. 8A-8B, the device 800 may include at least one contact 810 adapted to make contact with a portion of an oral cavity of a patient, and may include a control circuit 820 coupled to the at least one contact 810.

First, the device 800 may receive, from a number of the contacts 810 positioned within an oral cavity of a patient, a first signal indicative of one or more states of the patient's upper airway (1111). The first signal may be an EMG signal. In some aspects, the first signal is indicative of at least one of electrical activity in the patient's upper airway, movement of the patient's upper airway, and a change in the patient's respiration rate, and the second signal is an indication of at least one of an indication of an airflow in the patient's upper airway, an oxygenation rate, a level of carbon dioxide, a movement of the patient's chest, a movement of the patient's abdomen, and sounds emanating from the patient's upper airway.

The control circuit 820 may predict an onset of an apnea or disordered breathing in the patient based, at least in part, on one or more characteristics of the first signal at a first time (1112). For some implementations, the control circuit 820 may determine whether a magnitude of the first signal varies by more than an amount during a time period (1112A).

Then, the control circuit 820 may, prior to the onset of the apnea or disordered breathing, provide electrical stimulation via the number of contacts to one or more portions of the patient's upper airway based on the prediction of the onset of the apnea or disordered breathing (1113). Thereafter, the control circuit 820 may, after providing the electrical stimulation, detect a presence of apnea or disordered breathing in the patient based, at least in part, on one or more characteristics of the first signal at a second time (1114).

FIG. 11C is an illustrative flow chart depicting an example operation 1120 for monitoring a respiration of a patient, in accordance with some embodiments. The example operation 1120 is described below with respect to device 800 of FIG. 8A for simplicity only; the example operation 1120 may be performed by other suitable device. As described above with respect to FIGS. 8A-8B, the device 800 may include a number of contacts 810 adapted to make contact with a portion of an oral cavity of a patient, and may include a control circuit 820 coupled to the at least one contact 810.

First, the device 800 may receive, from a number of the contacts 810 positioned within an oral cavity of a patient, a first signal indicative of one or more states of the patient's upper airway (1121). In some aspects, the first signal is an EMG signal. In other aspects, the first signal is indicative of at least one of electrical activity in the patient's upper airway, movement of the patient's upper airway, and a change in the patient's respiration rate.

Then, the control circuit 820 may detect a change in respiration of the patient based, at least in part, on the first signal (1122). In some aspects, detecting the change in respiration may be based, at least in part, on a magnitude of the first signal varying by more than an amount during a time period.

For some implementations, the control circuit 820 may measure a first time period between first and second peaks in the first signal (1123), and may measure a second time period between third and fourth peaks in the first signal (1124). Then, the control circuit 820 may indicate an increase in the respiration if the first time period is greater than the second time period by more than a value (1125), and may indicate a decrease in the respiration if the first time period is less than the second time period by more than the value (1126).

The control circuit 820 may detect a hyperventilation or hypoventilation of the patient based, at least in part, on the first signal (1127). For some implementations, the control circuit 820 may measure a time period between first and second peaks of the first signal (1127A), may indicate hyperventilation of the patient if the time period is less than a value (1127B), and indicate hypoventilation of the patient if the time period is not less than the value (1127C).

The change in respiration of the patient may be accompanied by and/or indicative of a disordered breathing in the patient including, for example, a breathing obstruction (e.g., apnea), respiratory distress (e.g., CNS depression), and/or snoring. The change in respiration of the patient may also be accompanied by and/or indicative of a change in heartrate, blood pressure, inspiration effort, oxygen saturation levels, and so on.

FIG. 11D is an illustrative flow chart depicting an example operation 1130 for detecting an onset of snoring of a patient, in accordance with some embodiments. The example operation 1130 is described below with respect to device 800 of FIG. 8A for simplicity only; the example operation 1130 may be performed by other suitable device. As described above with respect to FIGS. 8A-8B, the device 800 may include a number of contacts 810 adapted to make contact with a portion of an oral cavity of a patient, and may include a control circuit 820 coupled to the at least one contact 810.

First, the device 800 may monitor a state of a patient's upper airway using a number of contacts positioned at least partially within the upper airway (1131). For some implementations, the contacts 810(1)-810(2) may provide one or more signals indicative of the monitored state (1131A). Then, the device 800 may detect an onset of snoring in the patient based, at least in part, on the monitored state (1132). In some aspects, the device 800 may indicate the onset of snoring based, at least in part, on an amplitude of at least a selected one of the signals associated of the upper airway (1132A).

The device 800 may electrically stimulate, via the number of contacts, at least a portion of the patient's upper airway based on the monitored state (1133). As discussed above, the device 800 may electrically stimulate one or more portions of the patient's upper airway to maintain upper airway patency.

The device 800 may limit a movement of the patient's jaw using a jaw stabilizer (1134). The device 800 may monitor a movement of the patient's jaw (1135). The device 800 may monitor a vibration of the patient's upper airway (1136). The device 800 may monitor a sound of the patient's upper airway (1137).

FIG. 11E is an illustrative flow chart depicting an example operation 1140 for determining a level of consciousness of a patient, in accordance with some embodiments. The example operation 1150 is described below with respect to device 800 of FIG. 8A for simplicity only; the example operation 1150 may be performed by other suitable device. As described above with respect to FIGS. 8A-8B, the device 800 may include a number of contacts 810 adapted to make contact with a portion of an oral cavity of a patient, and may include a control circuit 820 coupled to the at least one contact 810.

First, the device 800 may receive, from a number of contacts 810 positioned within an oral cavity of a patient, a first signal indicative of one or more states of the patient's upper airway (1141). In some aspects, the first signal is one or more electromyogram (EMG) signals. In other aspects, the first signal is indicative of at least one of electrical activity in the patient's upper airway, movement of the patient's upper airway, and a change in the patient's respiration rate.

The device 800 may determine a level of sleep or a level of consciousness in the patient based, at least in part, on the first signal (1142). The control circuit 820 may then indicate whether the level of sleep is relatively light or relatively deep (1143). For some implementations, the control circuit 820 may indicate a relatively light level of sleep based, at least in part, on the magnitude of the first signal varying by more than a first amount during a time period (1143A), and may indicate a relatively deep level of sleep based, at least in part, on the magnitude of the first signal varying by less than a second amount during the time period, wherein the second amount is less than the first amount (1143B). For other implementations, the control circuit 820 may indicate a relatively light level of sleep based, at least in part, on a duration of time between successive peaks of the first signal being greater than a first time period (1143C), and may indicate a relatively deep level of sleep based, at least in part, on the duration of time between successive peaks of the first signal being less than a second time period (1143D).

Then, the control circuit 820 may detect a change in the level of sleep based, at least in part, on the first signal (1144), and may selectively adjust an electrical stimulation provided to a portion of the patient's upper airway, via the number of contacts, based on the detected change in the level of sleep in the patient (1145).

Then, the device 800 may determine a level of consciousness of the patient based, at least in part, on the monitored state (1142). In some aspects, the device 800 may determine an average magnitude of a selected one of the signals during a time period (1142A), may indicate an increasing level of consciousness based, at least in part, on the average magnitude being greater than a threshold value (1142B), and may indicate a decreasing level of consciousness based, at least in part, on the average magnitude being less than the threshold value (1142C).

The device 800 may detect a change in the level of consciousness based, at least in part, on the one or more signals (1143). The device 800 may generate an alert based on the detected change in the level of consciousness (1143). The device 800 may transmit the alert to a remote device (1144).

FIG. 11F is an illustrative flow chart depicting an example operation 1150 for determining a level of compliance of a patient, in accordance with some embodiments. The example operation 1150 is described below with respect to device 800 of FIG. 8A for simplicity only; the example operation 1150 may be performed by other suitable device. As described above with respect to FIGS. 8A-8B, the device 800 may include a number of contacts 810 adapted to make contact with a portion of an oral cavity of a patient, and may include a control circuit 820 coupled to the at least one contact 810.

First, the device 800 may receive, from a number of contacts positioned within an oral cavity of a patient, a first signal indicative of one or more states of the patient's upper airway (1151). In some aspects, the first signal comprises an indication of one or more states of the patient's upper airway. In other aspects, the first signal is indicative of at least one of electrical or muscular activity in the patient's upper airway, movement in the patient's upper airway, and a change in the patient's respiration rate.

The device 800 may determine a presence of disordered breathing in the patient based, at least in part, on the first signal (1152), and then provide electrical stimulation to a portion of the patient's upper airway, via the number of contacts, based on the presence of disordered breathing in the patient (1153), for example, as described above with respect to FIGS. 8A-8B, 9A9E, and 10A-10D.

Then, the device 800 may determine a level of compliance of the patient's use of the device based, at least in part, on the first signal (1154). For some implementations, the control circuit 820 may determine an impedance level between at least two of the contacts (1154A), and then determine whether the device is located at least partially within the patient's oral cavity based, at least in part, on the determined impedance level (1154B).

Thereafter, the device 800 may detect a state of compliance based on the determined impedance level being less than a value (1155), and may detect a state of non-compliance based on the determined impedance level being greater than or equal to the value (1156).

FIG. 11G is an illustrative flow chart depicting an example operation 1160 for determining a type of disordered breathing in a patient, in accordance with some embodiments. The example operation 1160 is described below with respect to device 800 of FIG. 8A for simplicity only; the example operation 1160 may be performed by other suitable device. As described above with respect to FIGS. 8A-8B, the device 800 may include a number of contacts 810 adapted to make contact with a portion of an oral cavity of a patient, and may include a control circuit 820 coupled to the at least one contact 810.

First, the device 800 may receive, from a number of contacts positioned within an oral cavity of a patient, a first signal indicative of one or more states of the patient's upper airway (1161). In some aspects, the first signal comprises an indication of one or more states of the patient's upper airway. In other aspects, the first signal is indicative of at least one of electrical or muscular activity in the patient's upper airway, movement in the patient's upper airway, and a change in the patient's respiration rate.

Then, the device 800 may determine a type of disordered breathing in the patient based, at least in part, on the first signal (1162). The determined type of disordered breathing is one of a breathing obstruction, central nervous system (CNS) depression, and hyperventilation. For some implementations, the control circuit 820 may indicate a breathing obstruction based on a magnitude of the first signal decreasing during each of at least two successive respiratory cycles of the patient (1162A), and may indicate a central nervous system (CNS) depression based on the magnitude of the first signal remaining substantially constant for a number of respiratory cycles of the patient (1162B). For other implementations, the control circuit 820 may indicate a breathing obstruction based on a positive slope of the first signal decreasing during each of at least two successive respiratory cycles of the patient (1162C), and may indicate CNS depression based on the magnitude of the first signal remaining substantially constant for a number of respiratory cycles of the patient (1162D). For other implementations, the control circuit 820 may indicate a breathing obstruction based on a magnitude of the first signal varying by more than an amount during a time period (1162E), and may indicate CNS depression based on the magnitude of the first signal remaining relatively constant for the time period (1162F).

Thereafter, the device 800 may selectively provide electrical stimulation to a portion of the patient's upper airway, via the number of contacts, based on the determined type of disordered breathing (1163). For some implementations, the control circuit 820 electrically stimulates the portion of the patient's upper airway based on the disordered breathing being a breathing obstruction (1163A), and withholds electrical stimulation of the portion of the patient's upper airway based on the disordered breathing being CNS depression (1163B).

The example embodiments described above with respect to FIGS. 8A-8B, 9A-9E, 10A-10E, and 11A-11G may be used in hospitals, dental offices, and any other medical facility where anesthesia is administered or patients' airways must be monitored and/or managed will stock the device.

For actual embodiments, the device 800 may offer different standard mouthpiece sizes in order to get the best fit for the varying sizes of patients' mouths. These mouthpieces and the sizing conventions may be similar to dental impression trays currently in existence. The fit of the mouthpiece may be customized using dental wax or a similar material that easily conforms to any patient's mouth.

For acute care applications (e.g., prior to a surgical procedure), the device 800 may be placed in the patient's mouth before anesthesia is administered. The fit and placement may be handled by the nursing staff, physicians, or any other qualified personnel. The mouthpiece may have a small handle protruding from the mouth for quick and easy removal. This handle may also help prevent patients from aspirating or swallowing the device. The device 800 may include one or more electrical wires connecting to the electrical contacts on the mouthpiece. These electrical contacts may be permanently attached to the mouthpiece (e.g., for one-time use devices). The wires may also help prevent device aspiration/swallowing and assist in the removal of the device, if necessary. The wires from the electrical contacts connect to a monitoring station located in the same room. This station may be portable so that it can stay with the patient if they move between rooms in the hospital. This station may contain all of the circuitry required for stimulation, sensing, data processing, alarms/alerts, etc. This station may be an independent system or it may be integrated into an existing monitoring system.

Once the device 800 is placed in the mouth and plugged into the monitoring station, it may automatically begin sensing. The device 800 may perform an electrical or mechanical check to ensure that both contacts are in proper contact with the tissues of the upper airway. The acute care device may use EMG, capacitance, or any other sensing technology to monitor one or more states of a patient's upper airway and/or respiratory activity.

The device 800 may continue monitoring the patient as sedation begins. The device 800 may accurately indicate when a patient is fully sedated due to the physiological effects of anesthesia. During the onset of sedation, the tongue and diaphragm are the last two muscles of the human body to stop firing (vice versa during wakeup). Data provided by the device 800 may also be used by anesthesiologists to better administer anesthesia to patients and/or to ensure that patients are maintained at the right level of sedation throughout the procedure and recovery. This promotes safe sedation and may help identify the effects of certain medications.

When the device 800 detects or predicts an airway collapse, increase/decrease in respiratory rate, increase/decrease in respiratory effort, or any other issue, the monitoring station may alert/alarm the medical staff. These alerts/alarms may be in the form of a noise (beeping), a flashing LED light, an on-screen alert, etc.

If required, the device 800 may also activate to open a patient's airway and restart breathing. This therapeutic stimulation may be triggered automatically. If an alarm is ignored for a certain period of time or if the condition worsens, etc., the device 800 may be activated manually by a controller on the monitoring station. Anesthesiologists, their assistants, and any other qualified medical staff may trigger stimulation. Stimulation may also be applied to assist with an intubation procedure or to assist with Laryngospasm prevention.

If the device 800 determines that a patient has lost central respiratory drive, the monitoring station may issue a special alert/alarm. This will notify the medical staff to begin ventilation, as stimulating the airway may not help in this instance. This capability eliminates potential guesswork that typically goes on when a patient develops a respiratory issue in the acute care setting.

The device may remain in the mouth (or be reinserted if it was removed at some point) after the procedure is completed. The patient is still vulnerable as they are regaining consciousness and coming out of sedation. The device 800 may continue to monitor throughout the recovery process, for example, until the anesthesiologist has cleared the patient to go home.

At this point, the device 800 may be removed from the patient's mouth by grasping the handle and gently removing the device. The device 800 may be unplugged from the monitoring station and disposed of (single use). Any data collected during the device's use and stored in memory at the monitoring station may be uploaded to a local or cloud-based database and distributed accordingly. The monitoring station may be reset for immediate use with the next patient.

The device 800 may have the capability to identify the patient by their electrical signature and, in addition, will be able to support a single use limiter to ensure that the device is not re-used and potentially spread disease.

For snoring applications, device 800 may offer different standard mouthpiece sizes based on the size of the customers' mouths. Customers may be able to achieve a more customized fit by boiling their devices (similar to athletic mouth guards) or using an adhesive/mold technology. For high-end models, custom-fitted units may be recommended. A customer may insert the device 800 into their mouth before going to bed. The device 800 may be turned on by a physical switch, a smart phone app (via Bluetooth), or automatically based on its sensing capabilities. In some aspects, the device 800 may be wireless. The device 800 may always be “on” (e.g., constant stimulation).

The device 800 may also employ sensing capabilities so therapy is only delivered when necessary. Snoring can be sensed by a pressure sensor, microphone, accelerometer, EMG, capacitance, etc. Therapeutic electrical stimulation may be delivered to the patient's Palatoglossus muscle by at least one of contacts 810. These contacts 810 may be permanently connected to the device 800 or may be disposable. The electrical stimulation stabilizes the tongue and/or soft palate, thereby drastically reducing audible snoring and tissue vibration.

The device 800 may also have data collection capabilities. This data may be communicated directly to the patient via smart phone application, website, etc. The data may also be compiled in the Airway Analytics™ database. Airway Analytics™ may be the largest sleep database in the world and may provide a platform for data scientists to analyze thousands of patients' sleep data. Pertinent data may include the number of times the device had to intervene throughout the night, the customer's body and/or head position vs. snoring, overall quality of sleep, raw sleep waveforms and more. Data may also be transferred to physicians and insurers to monitor device efficacy, patient health, and compliance.

For OSA applications, doctors and dentists may prescribe the device 800 after patients have been diagnosed with OSA. Dentists, orthodontists, or any other qualified dental professional may conduct the specific sizing for the device 800. These contacts 810 may come in a variety of standard sizes or may be customized per patient.

Once a patient has been fitted for a mouthpiece, the on-board electronics will be integrated into the assembly. The dental professional that created the initial mouthpiece may complete this integration or the mouthpiece may be sent away to an off-site facility for the electronics to be added.

Upon receiving the completed device, patients will begin to use the device nightly. The device 800 may be placed in the mouth prior to going to bed each night (e.g., in a manner similar to a retainer). The device 800 may be turned on by a physical switch, a smart phone app (via Bluetooth, NFC, etc.), or automatically based on its sensing capabilities.

Capacitance sensing may be used to monitor the airway by itself or in combination as therapeutic stimulation is being administered. Once the device determines the patient has regained airway patency, stimulation will stop and the device will continue to monitor the status of the airway.

When a patient wakes up in the morning, the patient may remove the device 800 from the oral cavity and then place the device 800 on a suitable charging unit to charge the device 800. Charging may take place via a wired plug-in connector, wireless charging, or by swapping rechargeable battery packs. Additional technologies for power may be super capacitors, piezo electric current generators, galvanic cells, and so on.

The device 800 may also perform data transfer to the charging system to decrease onboard storage requirements. The data may immediately transfer to the cloud and/or be analyzed to provide the patient rapid feedback on how well they slept on a LCD, plasma display, LEDs, and so on.

The device 800 may be worn all day if desired. Rationale might be to build more data, increase airway performance, and improve long-term muscle tone. Alternate uses may be treatment of asthma patients, MS patients, stroke patients, COPD patients, and so on. The device 800 may provide treatment for various swallowing disorders such as dysphagia, dysphasia, and so on.

The device 800 may have sophisticated data collection capabilities. This data may be communicated directly to the patient via on board GUI, LEDs, a smart phone application, website, etc. The data may be compiled in the Airway Analytics™ database, which may provide a platform for data scientists to analyze thousands of patients' sleep data. Pertinent data may include the number of times the device had to intervene throughout the night, the customer's body and/or head position vs. snoring, overall quality of sleep, raw sleep waveforms and more. Data may also be transferred to physicians and insurers to monitor device efficacy, patient health, and compliance.

Long-term upper airway vibration reduction/prevention may prevent physical damage to tissue and nerves of the upper airway. The constant physical vibration to tissue during snoring may result in decreased upper airway muscle tone and increased snoring and apnea severity. Reducing vibration may prevent patients' breathing conditions from worsening over time.

In the foregoing specification, the example embodiments have been described with reference to specific example embodiments thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

What is claimed is:

1. A device comprising:
at least one contact adapted to make contact with a portion of an oral cavity of a patient; and
a control circuit coupled to the at least one contact and configured to:
in a first mode, detect a presence of disordered breathing in the patient based, at least in part, on a first signal received from the at least one contact; and
in a second mode, provide electrical stimulation via the at least one contact to a portion of the patient's upper airway based on a detection of the presence of disordered breathing.

2. The device of claim 1, wherein the first signal comprises one or more electromyogram (EMG) signals.

3. The device of claim 1, wherein the first signal is indicative of at least one of electrical or muscular activity in the patient's upper airway, movement in the patient's upper airway, and a change in the patient's respiration rate.

4. The device of claim 1, wherein the control circuit is configured to detect the presence of disordered breathing based on a magnitude of the first signal varying by more than an amount during a time period.

5. The device of claim 1, wherein the control circuit is configured to detect the presence of disordered breathing based on a positive slope of the first signal decreasing during each of at least two successive respiratory cycles of the patient.

6. The device of claim 1, wherein the control circuit is configured to detect the presence of disordered breathing based on a magnitude of the first signal decreasing during a respiratory cycle of the patient.

7. The device of claim 1, wherein the control circuit is configured to instruct, during the first mode, a waveform generator to send one or more stimulation waveforms to the at least one contact.

8. The device of claim 7, wherein the one or more stimulation waveforms cause the electrical stimulation to target the patient's Palatoglossus muscle without targeting the patient's Hypoglossal nerve.

9. The device of claim 1, wherein the control circuit is configured to determine a level of compliance of the patient using the device.

10. The device of claim 9, wherein the level of compliance is determined based on a sensed impedance between a pair of contacts.

11. The device of claim 1, wherein the control circuit is further configured to:
receive second signals from one or more sensors; and
detect the presence of disordered breathing based on a combination of the first and second signals.

12. The device of claim 11, wherein the one or more sensors includes at least one of a thermistor, an airflow detector, a thermocouple, or other temperature sensing devices.

13. The device of claim 11, wherein the first signal comprises an indication of one or more states of the patient's upper airway, and the second signals comprise an indication of airflow in the patient's upper airway.

14. The device of claim 13, wherein the control circuit is configured to indicate the presence of disordered breathing in the patient based on the second signals indicating a presence of airflow in the patient's oral cavity.

15. The device of claim 13, wherein the control circuit is configured to indicate a presence of central nervous system (CNS) depression based on the second signals indicating an absence of airflow in the patient's oral cavity.

16. The device of claim 1, wherein the control circuit is further configured to:
in a third mode, adjust one or more characteristics of the stimulation based on changes in the first signal resulting from a number of applications of stimulation to the patient's upper airway.

17. The device of claim 1, wherein the control circuit is further configured to:
in a fourth mode, provide electrical stimulation via the number of contacts to a portion of the patient's upper airway irrespective of the presence of disordered breathing.

18. A method of treating disordered breathing, comprising:
in a first mode, detecting a presence of disordered breathing in the patient based, at least in part, on a first signal received from at least one contact positioned within an oral cavity of a patient; and
in a second mode, providing electrical stimulation via the at least one contact to a portion of the patient's upper airway based on a detection of the presence of disordered breathing.

19. The method of claim 18, wherein the at least one contact is adapted to make contact with the patient's upper airway.

20. The device of claim 18, wherein the first signal comprises one or more electromyogram (EMG) signals.

21. The method of claim 18, wherein the first signal is indicative of at least one of electrical or muscular activity in the patient's upper airway, movement in the patient's upper airway, and a change in the patient's respiration rate.

22. The method of claim 18, wherein detecting the presence of disordered breathing comprises determining whether a magnitude of the first signal varies by more than an amount during a time period.

23. The method of claim 18, wherein detecting the presence of disordered breathing comprises determining whether a positive slope of the first signal decreases during each of at least two successive respiratory cycles of the patient.

24. The method of claim 18, wherein detecting the presence of disordered breathing comprises determining whether a magnitude of the first signal decreases during a successive respiratory cycle of the patient.

25. The method of claim 18, further comprising:
instructing, during the first mode, a waveform generator to send one or more stimulation waveforms to the at least one contact.

26. The method of claim 25, wherein the one or more stimulation waveforms cause the electrical stimulation to target the patient's Palatoglossus muscle without targeting the patient's Hypoglossal nerve.

27. The method of claim 18, further comprising:
determining a level of compliance of the patient using a device.

28. The method of claim 27, wherein determining the level of compliance comprises sensing a level of impedance between a pair of contacts.

29. The method of claim 18, further comprising:
receiving second signals from one or more sensors; and
detecting the presence of disordered breathing based on a combination of the first and second signals.

30. The method of claim 29, wherein the one or more sensors includes at least one of a thermistor, an airflow detector, a thermocouple, or other temperature sensing devices.

31. The method of claim 29, wherein the first signal comprises an indication of one or more states of the patient's upper airway, and the second signals comprise an indication of airflow in the patient's upper airway.

32. The method of claim 18, further comprising:
indicating the presence of disordered breathing based on the second signals indicating a presence of airflow in the patient's oral cavity.

33. The method of claim 18, further comprising:
indicating a presence of central nervous system (CNS) depression based on the second signals indicating an absence of airflow in the patient's oral cavity.

34. The method of claim 18, further comprising:
in a third mode, adjusting one or more characteristics of the stimulation based on changes in the first signal resulting from a number of applications of stimulation to the patient's upper airway.

35. The method of claim 18, further comprising:
in a fourth mode, providing electrical stimulation via the number of contacts to a portion of the patient's upper airway irrespective of the presence of disordered breathing.
